SEQUENCE LISTING (11 sequences)

SEQ ID NO: 1 - protein, 30 aa, synthetic construct
WEARLARALARALARHLARALARALRACEA

SEQ ID NO: 2 - RNA, 477 nt, synthetic construct
  source 1..477: mol_type = other RNA
  modified_base 1..477: mod_base = m1f
atgcaccagaagcggaccgccatgttccaggacccccaggagagacctagaaagctgcctcagctgtgcaccgagctgcagaccaccatccacgacatcatcctggaatgcgtgtattgtaaacagcaactgctgagacgggaagtgtacgacttcgccttcagagatctgtgcatcgtgtacagagacggcaacccctacgccgtgtgcgataagtgcctgaagttctacagcaagatcagcgagtaccggcactactgctacagcctgtacggcaccacactggaacagcagtacaacaagcccctgtgcgacctgctgatccggtgcatcaactgccagaaacctctgtgtcctgaggaaaagcagagacacctggacaagaagcagcgatttcacaacatcagaggaagatggacaggcagatgcatgagctgctgcagatctagcagaaccagaagagagacacagctgtga

SEQ ID NO: 3 - RNA, 297 nt, synthetic construct
  source 1..297: mol_type = other RNA
  modified_base 1..297: mod_base = m1f
atgcacggcgacacccctacactgcacgagtacatgctggacctgcagcctgaaaccaccgacctgtactgctacgagcagctgaacgactctagcgaggaagaggacgagatcgacggccctgccggccaggccgagcctgatagagcccactacaacatcgtgaccttctgctgcaagtgcgacagcacccttagactgtgcgtgcagagcacacacgtggacatcagaaccctggaagatctgctgatgggcaccttgggcatcgtgtgccccatctgcagccagaagccttga

SEQ ID NO: 4 - RNA, 1950 nt, synthetic construct
  source 1..1950: mol_type = other RNA
  modified_base 1..1950: mod_base = m1f
atggcggaccccgctggcaccaacggagaagaaggcaccggctgcaacggctggttctacgtggaagccgtggtggaaaagaagaccggcgacgccatcagcgacgacgagaatgagaacgatagcgacaccggcgaggatctcgtcgatttcatagtcaatgataacgattacctgacgcaggccgaaacagaaacggcccatgcgctgttcaccgcacaagaggcgaagcagcatagagatgcagtgcaagtattgaaaaggaagtatttggtgagccctctgtctgacataagcggatgtgtggacaataatataagcccacgccttaaggcaatctgtatcgagaagcagagcagggccgcgaagaggcggctgttcgagagcgaagacagcggatatggcaataccgaagtggagacacagcaaatgttgcaagtagaaggccgccacgagacagaaaccccctgtagccagtatagcggtggtagcggaggtggatgtagccagtacagcagcggaagcggcggcgaaggggttagcgagagacataccatctgccaaactccgctgacgaacattttgaatgtgctcaagacgagcaacgccaaagctgcaatgcttgcgaagtttaaagaattgtacggcgttagctttagcgaattggtgagacccttcaagagcaataagagcacttgttgtgattggtgcatcgctgcatttggccttacaccgagcattgcggacagcataaagactttgctgcaacagtactgtctgtacctgcacattcagagcctcgcatgtagctggggaatggtcgtgctgctcctggtgcgatacaagtgcggaaaaaatagagaaactatagagaagctcctcagcaagctgctctgcgttagcccaatgtgcatgatgattgagcccccaaagttgcgaagcaccgccgccgcactttactggtacaagactggtataagcaacatcagcgaagtgtacggagatacacccgagtggatccagcgccaaaccgttctccagcatagctttaacgactgtacgtttgaattgagccagatggtccagtgggcgtatgacaacgatatcgttgacgactcagaaatagcttacaaatacgcacagctcgcggacaccaacagcaacgcaagcgcgtttttgaagagtaacagccaagccaagatcgttaaggattgtgcgactatgtgtagacactataagcgcgctgagaagaaacaaatgtcaatgagccaatggattaaataccgatgtgatcgggttgatgacggtggcgattggaagcaaattgtcatgtttcttagatatcagggggttgagttcatgagcttcttgacagcgctcaagagatttctgcaagggatcccaaagaagaattgtatactcctttacggagctgccaatacagggaagagtctgttcggaatgagcctgatgaaattcctccaagggagcgtcatctgctttgtcaacagcaaaagccacttttggcttcaacctctcgccgatgcgaaaattggtatgctcgatgatgctaccgtcccatgctggaactacatcgacgataaccttagaaatgcacttgacgggaatctggtgagcatggatgttaagcacagacctcttgtgcagttgaaatgccctccacttctcatcacatcaaatattaacgcggggacggacagccgatggccctatctgcacaatcggcttgtggtgttcacgttcccaaatgagttccctttcgacgagaacggaaatccagtatatgaactgaatgacaaaaactggaagagtttcttctcccggacctggagcagactgagcctgcatgaggacgaggataaagaaaatgacggggacagcctccccactttcaaatgcgtctctggacagaacactaacaccttgtaa

SEQ ID NO: 5 - protein, 158 aa, synthetic construct
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 6 - protein, 98 aa, synthetic construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 7 - protein, 649 aa, synthetic construct
MADPAGTNGEEGTGCNGWFYVEAVVEKKTGDAISDDENENDSDTGEDLVDFIVNDNDYLTQAETETAHALFTAQEAKQHRDAVQVLKRKYLVSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTL

SEQ ID NO: 8 - RNA, 489 nt, synthetic construct
  source 1..489: mol_type = other RNA
  modified_base 1..489: mod_base = m1f
atgagaatcagcaagccgcacctgagaagcatcagcattcagtgctacctctgcctgctgctcaacagccacttcctgaccgaagccggaatccacgtattcatcctgggatgcttcagcgccggcctccccaaaacagaggcaaactgggtaaacgtaataagcgacctcaaaaagatcgaagacctgatccagagcatgcacatcgacgcaaccctgtacaccgaaagcgacgtacacccaagctgcaaagtaaccgcgatgaaatgcttcctgctggagctgcaagtaatcagcctggagagcggcgacgccagcatccacgacaccgtggaaaacctgattatcttggcaaacaacagcctgagcagcaacggaaacgtgacagagagcggatgcaaagagtgcgaggagctggaggaaaagaacatcaaagaattcctccaaagcttcgtgcacatagtgcagatgttcataaacacaagctga

SEQ ID NO: 9 - RNA, 435 nt, synthetic construct
  source 1..435: mol_type = other RNA
  modified_base 1..435: mod_base = m1f
atgtggctgcaaagccttctgctcttggggactgtggcctgctcaatttctgcacctgcccgatcgcccagtccaagcacgcagccctgggagcatgtgaatgccatccaggaggcgcggaggttactgaacctgagtagagacactgctgctgagatgaatgaaacagtagaagtgatatcagaaatgtttgatctccaggagcctacctgcctacagacacgcctggagctgtataaacagggcctgaggggctccctcaccaagctcaaaggacccttgacaatgatggccagccactacaagcaacactgtcctccaaccccggaaacttcctgtgcaacccagattatcacatttgaatccttcaaagagaacctgaaggatttcctacttgttatccccttcgactgctgggagccagtccaggagtga

SEQ ID NO: 10 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 144 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

(19) United States
(12) Patent Application Publication
(10) Pub. No.: US 2025/0177512 A1
(43) Pub. Date: Jun. 5, 2025

(54) mRNA VACCINE

(71) Applicant: PHION THERAPEUTICS LTD, Belfast (IE)

(72) Inventors: Helen MCCARTHY, Belfast (IE); Cian Michael MCCRUDDEN, Belfast (IE)

(21) Appl. No.: 18/838,972

(22) PCT Filed: Feb. 17, 2023
(86) PCT No.: PCT/EP2023/054037

§ 371 Date: Aug. 15, 2024

(30) Foreign Application Priority Data
Feb. 18, 2022 (GB) 2202186.9

(51) Int. Cl. A61K 39/12; A61K 9/00; A61K 9/51; A61K 39/00; A61K 47/26; A61P 35/00; C12N 7/00

(52) U.S. Cl. CPC A61K 39/12; A61K 9/0019; A61K 9/5169; A61K 47/26; A61P 35/00; C12N 7/00; A61K 2039/53; A61K 2039/55516; A61K 2039/55555; C12N 2710/20034

(57) ABSTRACT

The present specification relates to vaccines comprising an mRNA polynucleotide encoding an antigen from an infectious microorganism; and an amphipathic cell penetrating RALA peptide.

### FIELD

The present specification relates to vaccines. In particular, the specification relates to vaccines comprising an mRNA polynucleotide encoding an antigen from an infectious microorganism, particularly an HPV antigen, and an amphipathic cell penetrating peptide from the RALA family of peptides. The specification further relates to methods of preparing such vaccines and to their use in therapy.

### BACKGROUND

Vaccines involve introducing antigens—a substance that the immune system will attack—either directly or indirectly into the body, typically via injection. Vaccines fall into two broad categories: prophylactic and therapeutic. Prophylactic vaccines are designed to build immunity—the antigens provoke the body's immune system to create antibodies and a cellular response to protect against future infection and/or disease. Therapeutic vaccines are designed to help the body do a better job of fighting an infection and/or disease it already has.

To produce a therapeutic vaccine, a particular type of immune response, known as a CD8+ response, is required. CD8+ T cells are cytotoxic T cells of the immune system. They are provoked on encountering a pathogenic stimulus, and kill cells presenting foreign immunogenic antigen fragments. CD8+ T-cells are a critical component of the cellular immune response and play an important role in the control of viral infection. CD8+ T-cells are able to recognize and destroy infected cells, as well as suppress viral binding and transcription. A CD8+ mediated response is considered essential to treat active, latent, and persistent viral pathogens (for example Cosma et al., 2018 Apr. 27; 7: F1000 Faculty Rev-508, Beura et al., Immunity, 2018 Feb. 20; 48 (2): 327-338.e5, and McMichael, Cold Spring Harb. Perspect. Biol., 2018 Sep. 4; 10 (9): a029124), and requires specific delivery of the antigen into the antigen presenting cells (Gessani et al., Toxins (Basel), 2014 May 26; 6 (6): 1696-723). Current antigenic protein vaccines delivered via intramuscular injection utilise a CD4+ mediated approach with adjuvants, to produce either a cytolytic CD4+ immune responses or antigen cross-presentation to elicit a CD8+ response. CD4+ T cells are “helper” cells, they regulate the immune response to a particular antigen and operate more indirectly than CD8+ T-cells. A vaccine provoking a CD8+ dominated response has remained elusive due to the triggering of innate, cellular immunity, and an inability to deliver the antigen to the right location within the right immune cells.

There are several types of vaccines, including inactivated vaccines; live-attenuated vaccines; messenger ribonucleic acid (mRNA) vaccines; subunit, recombinant, polysaccharide, and conjugate vaccines; toxoid vaccines; and viral vector vaccines. These different vaccines introduce the antigen into the body in different ways.

mRNA vaccines convey instructions to cells to make antigens, which in turn provoke an immune response. The mRNA selected encodes for an antigen of the infection and/or disease to be treated. mRNA vaccines have several benefits compared to other types of vaccines including shorter manufacturing times, and since mRNA cannot be incorporated into the cellular genome, they carry no risk of causing the disease in the person getting vaccinated. However, unmodified synthetic mRNA is not stable, and the mRNA itself can stimulate the activation of an unwanted (e.g. CD4+) immune response. Chemical modifications on the ribose, the RNA termini, and nucleobases may therefore be required to improve stability and reduce immunogenicity (see for example Gao et al., Acta. Biomater. 2021 Sep. 1; 131:1-15).

Human papilloma virus (HPV) is the most common sexually transmitted disease, with an estimated 200 million people infected worldwide (WHO Global HPV Market Study 2018). Infection with HPV can lead to genital warts, precancerous lesions and cancer. Most HPV infections are subclinical and more than 90 percent of all new HPV infections are undetectable within two years, even without treatment. Unfortunately, some infections persist and lead to complications, and as an etiological agent, HPV is responsible for 5% of all cancers worldwide, including 99% of all cervical cancers and 60% of all cutaneous non-melanoma cancers such as basal cell and squamous cell (de Martel et al., New Microbiol. 2017 April; 40 (2): 80-85 and Brianti et al., Int J Cancer. 2017 Aug. 15; 141 (4): 664-670). HPV induced cervical cancer is the second most common cause of death in females after breast cancer.

Of the more than 150 strains of HPV, 40 affect the genital area, but most don't pose a serious risk to health. A person can be infected with more than one HPV strain at a time, and strains are identified by number. HPV strains are categorised as low-risk HPV or high-risk HPV. Infections with most low-risk HPV strains are asymptomatic, and these strains are not associated with cancer. Infections with high-risk HPV are more problematic, and can lead to cervical dysplasia and certain types of cancer. There are at least 12 high-risk strains of HPV, including HPV 16, HPV 18, HPV 31, HPV 33, HPV 35, HPV 39, HPV 45, HPV 51, HPV 52, HPV 56, HPV 58, and HPV 59, of which two-HPV 16 and HPV 18-cause the majority of HPV-related cancers.

Current HPV vaccines are prophylactic vaccines, designed to be administered to 11-14-year-olds to prevent infection (Cook et al., Pediatrics. 2018 September; 142 (3)). These vaccines have no effect on those already infected with HPV, and even if prophylactic coverage was universal, it would take at least 20 more years for it to translate into significant reduction of HPV-related morbidity (Kumar et al., Med J Armed Forces India, 2015 April; 71 (2): 171-7). Meanwhile, incidence rates for cervical cancer are predicted to rise by 43% in the UK alone, with 17 new cases per 100,000 females by 2035 (British Gynaecological Cancer Society (BGCS) Cervical Cancer Guidelines: Recommendations for Practice). There is clearly a need for a therapeutic vaccine, or vaccine capable of acting both therapeutically and prophylactically, to address existing HPV, particularly HPV 16 and HPV 18, infection.

There are several known HPV proteins (L1, L2, E1, E2, E4, E5, E6 and E7) that can function as antigens in a vaccination approach for the treatment of HPV infection. Prophylactic vaccines on the market utilize the major capsid (viral surface) protein L1 (Kirnbauer et al., Proc. Natl. Acad. Sci. USA, 1992 Dec. 15; 89 (24), and Schiller et al., Vaccine, 2018 Aug. 6; 36 (32 Pt A): 4768-4773). However, L1 (and L2) proteins get inactivated or deleted during the integration of the HPV genome into the cellular genome, and as a consequence, vaccines employing L1 and L2 antigens are ineffective against an HPV infection once it is established. The early proteins E1, E2, E4, E5, E6 and E7 are expressed for a longer period in the life cycle of HPV and represent a better target for therapeutic vaccines, even for advanced HPV infection (Pal et al., Front Microbiol. 2019; 10:3116).

The antigen delivery system employed in mRNA vaccination, plays a key role in determining the type of immune response. The ratio of carrier:mRNA also plays a role-mRNA molecules are negatively charged polynucleotides and the carrier needs both to protect the cargo from degradation as well as deliver it to the correct target cells. Optimisation of the carrier:mRNA ratio facilitates production of nanoparticles with suitable size and charge characteristics to ensure uptake by antigen-presenting cells.

The RALA family of peptides are amphipathic peptides composed of repeating RALA units that are capable of overcoming biological barriers to gene delivery, both in vitro and in vivo. The term “RALA” has been used inconsistently in the literature, but typically refers to an amphipathic peptide or group of peptides composed of repeating RALA units generally of less than approximately 50 amino acid residues. Cohen-Avrahami et al. (J. Phys. Chem. B 2011, 115:10 189-1 097 and Colloids and Surfaces B: Biointerfaces 77 (2010) 131-138) disclose an amphipathic 16-mer peptide referred to as “RALA”. Faranack et al., (Biomacromolecules 2013, 14, 2033-2040) use the term “RALA” to describe a 30-mer RALA peptide, as does Mccarthy et al. (Journal of Controlled Release 189 (2014) 141-149) but for a different 30-mer peptide. WO 2014/087023 and WO 2015/189205 defined the term “RALA” as a generic term for a group of peptides falling within the scope of the invention as described therein.

The RALA family of peptides have been used to deliver genetic material such as plasmid DNA (McCarthy H O et al. J Control Release, 2014 Sep. 10; 189:141-9; and Ali A A et al., Nanomedicine, 2017 April; 13 (3): 921-932), mRNA (Udhayakumar et al., Adv. Healthc. Mater. 2017 July; 6 (13)), siRNA (Mulholland E. J. et al., J Control Release, 2019 Dec. 28; 316:53-65), and small molecules such as bisphosphonates (Jena L N et al., J. Nanobiotechnology. 2021 May 4; 19 (1): 127), and calcium phosphates (Sathy B. N. et al., J. Mater. Chem. B. 2017 Mar. 7; 5 (9): 1753-1764).

The specific RALA peptide WEARLARALARALARHLARALARALRACEA (SEQ ID_No 1) is a 30 amino acid, non-toxic, peptide with a +6 electric charge at a physiological pH that converts to a +8 helical cell penetrating conformation at acidic conditions found inside the endosome of a cell. When complexed with certain payloads, e.g. DNA and mRNA, in water it has been shown to be capable of spontaneous self-assembly into nanoparticles (McCarthy et al. 2014 and Udhayakumar et al. 2017). This pH dependent change allows for escape of the peptide and the cargo within a cell, resulting in highly efficiency cellular entry and cargo delivery, without any associated toxicity at the physiological pH of 7.4 outside the cell. The nanoparticles have been shown to be extremely stable at a range of temperatures and over time, and RALA peptides do not themselves provoke an immunological response.

Udhayakumar et al. (Udhayakumar et al. 2017) evaluated the capacity of this RALA peptide in mRNA nanocomplexes to prime CD8+ T cells by immunising mice with RALA complexed mRNA encoding the model antigen ovalbumin (the main protein found in egg white). Udhayakumar et al. concluded that multiple different mRNA nucleotide modifications and a very high N/P ratio (i.e. ratio of positively charged nitrogen atoms in the peptide to negatively charged phosphates in the mRNA backbone) were needed for optimum complexation of Ova mRNA and to enable efficient CD8+ T cell priming.

Ali et al. (Ali et al. 2017) investigated a DNA vaccination for cervical cancer; delivering RALA condensed E6/E7 plasmid DNA nanoparticles via polymeric microneedles. However, in order for DNA vaccines to work, the cargo must reach the nucleus, be transcribed to mRNA, and the protein translated. mRNA vaccines remove the transcription step, and the need for the nucleic acid to reach the target cell nucleus.

Coolen et al (Biomaterials, vol. 195, 2019 al., p. 23-37) investigated the ability of poly(lactic acid) nanoparticles coupled with cell-penetrating peptides to potentiate mRNA-based vaccine expression in dendritic cells triggering their activation. However, a RALA peptide complexed with eGFP (Green Fluorescent Protein) failed to efficiently transfect DC2.4 dendritic cells and was not selected for further evaluation.

The present specification describes vaccines comprising an mRNA polynucleotide encoding an antigen from an infectious microorganism, particularly an HPV antigen, and a specific RALA peptide. These vaccines result in intracellular delivery of the mRNA cargo, protecting it from degradation, and do not provoke an innate (CD4+) immune response. Furthermore, the RALA complexed mRNA nanoparticles can be readily lyophilized (retaining functionality), are stable and readily reconstituted, do not require cold chain storage, and are relatively inexpensive to manufacture.

### SUMMARY

This specification describes, in part, a vaccine, comprising:
i) an mRNA polynucleotide comprising an open reading frame encoding an antigen from an infectious microorganism or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

This specification also describes, in part, vaccines as described herein for use in therapy, particularly for use in the treatment of viral infections and in the treatment of cancer.

### DETAILED DESCRIPTION OF THE INVENTION

Many embodiments of the invention are detailed throughout the specification and will be apparent to a reader skilled in the art. The invention is not to be interpreted as being limited to any of the recited embodiments.

“A” (or “an”) means “at least one”. In any embodiment where “a” is used to denote a given material or element, “a” may mean one.

“Comprising” means that a given material or element may contain other materials or elements. In any embodiment where “comprising” is mentioned the given material or element may be formed of at least 10% w/w, at least 20% w/w, at least 30% w/w, or at least 40% w/w of the material or element. In any embodiment where “comprising” is mentioned, “comprising” may also mean “consisting of” (or “consists of”) or “consisting essentially of” (or “consists essentially of”) a given material or element.

“Consisting of” or “consists of” means that a given material or element is formed entirely of the material or element. In any embodiment where “consisting of” or “consists of” is mentioned, the given material or element may be formed of 100% w/w of the material or element.

“Consisting essentially of” or “consists essentially of” means that a given material or element consists almost entirely of that material or element. In any embodiment where “consisting essentially of” or “consists essentially of” is mentioned the given material or element may be formed of at least 50% w/w, at least 60% w/w, at least 70% w/w, at least 80% w/w, at least 90% w/w, at least 95% w/w or at least 99% w/w of the material or element.

In any embodiment where “is” or “may be” is used to define a material or element, “is” or “may be” may mean the material or element “consists of” or “consists essentially of” the material or element.

Embodiments may be combined.

Claims are embodiments.

mRNA polynucleotide

A polynucleotide is a compound and/or substance that comprises a polymer of nucleotides (nucleotide monomers). The polynucleotides of the present disclosure function as messenger RNA (mRNA). “Messenger RNA” refers to any polynucleotide that encodes a polypeptide (both naturally-occurring (wild-type) and non-naturally-occurring) and can be translated to produce the encoded polypeptide in vitro, in vivo, in situ or ex vivo.

The basic components of an mRNA molecule typically include at least one open reading frame, a 5′ untranslated region (UTR), a 3′ UTR, a 5′ cap and a poly-A tail. The mRNA polynucleotides described herein function as mRNA but may differ from wild-type mRNA in both functional and/or structural design features.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

A “poly-A tail” is a region of mRNA that is downstream, e.g., directly downstream (i.e., 3′), from the 3′ UTR that contains multiple, consecutive adenosine monophosphates. A poly-A tail may contain 10 to 150 adenosine monophosphates. In one embodiment, a poly-A tail contains 50 to 150 adenosine monophosphates. In one embodiment, a poly-A tail contains 75 to 125 adenosine monophosphates. In one embodiment, a poly-A tail contains about 120 adenosine monophosphates. In one embodiment, a poly-A tail contains 120 adenosine monophosphates. In one embodiment, a poly-A tail contains about 80 adenosine monophosphates. In one embodiment, a poly-A tail contains 80 adenosine monophosphates. In a relevant biological setting (e.g., in cells, in vivo) the poly-A tail functions to protect mRNA from enzymatic degradation, e.g., in the cytoplasm, and aids in transcription termination, export of the mRNA from the nucleus, and translation.

In one embodiment the vaccine comprises an mRNA polynucleotide comprising an open reading frame encoding an antigen from an infectious microorganism or an immunogenic fragment thereof wherein the mRNA polynucleotide comprises 200 to 5,000 nucleotides.

In one embodiment the vaccine comprises an mRNA polynucleotide comprising an open reading frame encoding an antigen from an infectious microorganism or an immunogenic fragment thereof wherein the mRNA polynucleotide comprises 200 to 4,000 nucleotides.

In one embodiment the vaccine comprises an mRNA polynucleotide comprising an open reading frame encoding an antigen from an infectious microorganism or an immunogenic fragment thereof wherein the mRNA polynucleotide comprises 200 to 3,000 nucleotides.

In one embodiment the vaccine comprises an mRNA polynucleotide comprising an open reading frame encoding an antigen from an infectious microorganism or an immunogenic fragment thereof wherein the mRNA polynucleotide comprises 200 to 1,000 nucleotides.

In one embodiment the vaccine comprises an mRNA polynucleotide comprising an open reading frame encoding an antigen from an infectious microorganism or an immunogenic fragment thereof wherein the mRNA polynucleotide comprises 500 to 1000 nucleotides.

In one embodiment the vaccine comprises an mRNA polynucleotide comprising an open reading frame encoding an antigen from an infectious microorganism or an immunogenic fragment thereof wherein the mRNA polynucleotide comprises 500 to 800 nucleotides.

In one embodiment the vaccine comprises an mRNA polynucleotide comprising an open reading frame encoding an antigen from an infectious microorganism or an immunogenic fragment thereof wherein the mRNA polynucleotide comprises 753 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 753 nucleotides.

In one embodiment the vaccine comprises an mRNA polynucleotide comprising an open reading frame encoding an antigen from an infectious microorganism or an immunogenic fragment thereof wherein the mRNA polynucleotide comprises 573 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 573 nucleotides.

In one embodiment the vaccine comprises an mRNA polynucleotide comprising an open reading frame encoding an antigen from an infectious microorganism or an immunogenic fragment thereof wherein the mRNA polynucleotide comprises 703 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 703 nucleotides.

In one embodiment the vaccine comprises an mRNA polynucleotide comprising an open reading frame encoding an antigen from an infectious microorganism or an immunogenic fragment thereof wherein the mRNA polynucleotide comprises 522 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 522 nucleotides.

In one embodiment the vaccine comprises an mRNA polynucleotide comprising an open reading frame encoding an antigen from an infectious microorganism or an immunogenic fragment thereof wherein the mRNA polynucleotide comprises 2175 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 2175 nucleotides.

In one embodiment the vaccine comprises an mRNA polynucleotide comprising an open reading frame encoding an antigen from an infectious microorganism or an immunogenic fragment thereof wherein the mRNA polynucleotide comprises 2211 nucleotides.

In one embodiment the vaccine comprises an mRNA polynucleotide comprising an open reading frame encoding an antigen from an infectious microorganism or an immunogenic fragment thereof wherein the mRNA polynucleotide comprises about 2211 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 250-300 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 270-280 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise 276 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 276 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail consist of 276 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 200-300 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 210-280 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise 210-230 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail comprise about 210-230 nucleotides.

In one embodiment the vaccine comprises an mRNA polynucleotide comprising an open reading frame, a 5′ UTR, a 3′ UTR, a 5′ cap and a poly-A tail encoding an antigen from an infectious microorganism or an immunogenic fragment thereof wherein the 5′ UTR, the 3′ UTR, and the poly-A tail consist of 210-230 nucleotides.

### An Open Reading Frame

An “open reading frame” (ORF) is a continuous stretch of polynucleotide beginning with a start codon (e.g., methionine (AUG)), and ending with a stop codon (e.g., UAA, UAG or UGA). It encodes, and has the potential to be translated into, a polypeptide (in this case the antigen or the immunogenic fragment thereof). It is also known as a coding region.

In one embodiment the vaccine comprises an mRNA polynucleotide comprising an open reading frame encoding an antigen from an infectious microorganism or an immunogenic fragment thereof wherein the open reading frame comprises 250-2500 nucleotides.

In one embodiment the vaccine comprises an mRNA polynucleotide comprising an open reading frame encoding an antigen from an infectious microorganism or an immunogenic fragment thereof wherein the open reading frame comprises 250-500 nucleotides.

In one embodiment the vaccine comprises an mRNA polynucleotide comprising an open reading frame encoding an antigen from an infectious microorganism or an immunogenic fragment thereof wherein the open reading frame comprises 1950 nucleotides.

In one embodiment the vaccine comprises an mRNA polynucleotide comprising an open reading frame encoding an antigen from an infectious microorganism or an immunogenic fragment thereof wherein the open reading frame comprises about 1950 nucleotides.

In one embodiment the vaccine comprises an mRNA polynucleotide comprising an open reading frame encoding an antigen from an infectious microorganism or an immunogenic fragment thereof wherein the open reading frame comprises 477 nucleotides.

In one embodiment the vaccine comprises an mRNA polynucleotide comprising an open reading frame encoding an antigen from an infectious microorganism or an immunogenic fragment thereof wherein the open reading frame comprises about 477 nucleotides.

In one embodiment the vaccine comprises an mRNA polynucleotide comprising an open reading frame encoding an antigen from an infectious microorganism or an immunogenic fragment thereof wherein the open reading frame comprises 297 nucleotides.

In one embodiment the vaccine comprises an mRNA polynucleotide comprising an open reading frame encoding an antigen from an infectious microorganism or an immunogenic fragment thereof wherein the open reading frame comprises about 297 nucleotides.

In one embodiment the vaccine comprises an mRNA polynucleotide comprising an open reading frame encoding an antigen from an infectious microorganism or an immunogenic fragment thereof wherein the open reading frame comprises 45-90% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises 45-70% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises 50-65% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises 90% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises about 90% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises 63% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises about 63% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises 52% of the total nucleotides of the mRNA.

In one embodiment the vaccine comprises an mRNA polynucleotide comprising an open reading frame encoding an antigen from an infectious microorganism or an immunogenic fragment thereof wherein the open reading frame comprises about 52% of the total nucleotides of the mRNA.

In one embodiment the vaccine comprises an mRNA polynucleotide comprising an open reading frame encoding an HPV antigen or an immunogenic fragment thereof.

In one embodiment the vaccine comprises an mRNA polynucleotide comprising an open reading frame encoding an E1, E2, E4, E5, E6, E7, L1, or L2 HPV antigen or an immunogenic fragment thereof.

In one embodiment the vaccine comprises an mRNA polynucleotide comprising an open reading frame encoding an E1, E2, E4, E5, E6 or E7 HPV antigen or an immunogenic fragment thereof.

In one embodiment the vaccine comprises an mRNA polynucleotide comprising an open reading frame encoding an E6 or E7 HPV antigen or an immunogenic fragment thereof.

In one embodiment the vaccine comprises an mRNA polynucleotide comprising an open reading frame encoding an E1 or E6 or E7 HPV antigen or an immunogenic fragment thereof.

In one embodiment the vaccine comprises an mRNA polynucleotide comprising an open reading frame encoding an E1 HPV antigen or an immunogenic fragment thereof.

In one embodiment the vaccine comprises an mRNA polynucleotide comprising an open reading frame encoding an E6 HPV antigen or an immunogenic fragment thereof.

In one embodiment the vaccine comprises an mRNA polynucleotide comprising an open reading frame encoding an E7 HPV antigen or an immunogenic fragment thereof.

In one embodiment the vaccine does not comprise an mRNA polynucleotide comprising an open reading frame encoding an E5 HPV antigen or an immunogenic fragment thereof.

In one embodiment the vaccine comprises an mRNA polynucleotide comprising an open reading frame encoding an L1 or L2 HPV antigen or an immunogenic fragment thereof.

In one embodiment the vaccine comprises an mRNA polynucleotide comprising an open reading frame encoding an L1 HPV antigen or an immunogenic fragment thereof.

In one embodiment the vaccine comprises an mRNA polynucleotide comprising an open reading frame encoding an L2 HPV antigen or an immunogenic fragment thereof.

In one embodiment the vaccine comprises an mRNA polynucleotide comprising an open reading frame encoding a high-risk HPV antigen or an immunogenic fragment thereof.

In one embodiment the vaccine comprises an mRNA polynucleotide comprising an open reading frame encoding an HPV 16, 18, 31, 33, 35, 39, 45, 51, 52, 56, 58, 59, 66, and/or 68 antigen or an immunogenic fragment thereof.

In one embodiment the vaccine comprises an mRNA polynucleotide comprising an open reading frame encoding an HPV 6, 8, 11, 16 and/or 18 antigen or an immunogenic fragment thereof.

In one embodiment the vaccine comprises an mRNA polynucleotide comprising an open reading frame encoding an HPV 16 and/or 18 antigen or an immunogenic fragment thereof.

In one embodiment the vaccine comprises an mRNA polynucleotide comprising an open reading frame encoding a low-risk HPV antigen or an immunogenic fragment thereof.

In one embodiment the vaccine comprises an mRNA polynucleotide comprising an open reading frame encoding an HPV 6 and/or 11 antigen or an immunogenic fragment thereof.

In one embodiment the vaccine comprises an mRNA polynucleotide comprising an open reading frame encoding an E1 HPV 16 antigen or an immunogenic fragment thereof.

In one embodiment the vaccine comprises an mRNA polynucleotide comprising an open reading frame encoding an E1 HPV 18 antigen or an immunogenic fragment thereof.

In one embodiment the vaccine comprises an mRNA polynucleotide comprising an open reading frame encoding an E6 HPV 16 antigen or an immunogenic fragment thereof.

In one embodiment the vaccine comprises an mRNA polynucleotide comprising an open reading frame encoding an E6 HPV 18 antigen or an immunogenic fragment thereof.

In one embodiment the vaccine comprises an mRNA polynucleotide comprising an open reading frame encoding an E7 HPV 16 antigen or an immunogenic fragment thereof.

In one embodiment the vaccine comprises an mRNA polynucleotide comprising an open reading frame encoding an E7 HPV 18 antigen or an immunogenic fragment thereof.

Table 1 describes certain mRNA open reading frame sequences that encode antigens from infectious microorganisms that may be incorporated into the mRNA polynucleotides in the vaccines 10 described herein.

TABLE 1
mRNA polynucleotides encoding antigens

| | mRNA Open Reading Frame Sequence | |
|---|---|---|
| | (all uridines “U” are N1-methylpseudouridine | |
| Antigen | nucleotides) | SEQ ID_No |
| HPV 16 E6 | AUGCACCAGAAGCGGACCGCCAUGUUCCAGGACCCCCAGGAGAGACCUA | SEQ ID_No 2 |
| | GAAAGCUGCCUCAGCUGUGCACCGAGCUGCAGACCACCAUCCACGACAU | |
| | CAUCCUGGAAUGCGUGUAUUGUAAACAGCAACUGCUGAGACGGGAAG | |
| | UGUACGACUUCGCCUUCAGAGAUCUGUGCAUCGUGUACAGAGACGGC | |
| | AACCCCUACGCCGUGUGCGAUAAGUGCCUGAAGUUCUACAGCAAGAUC | |
| | AGCGAGUACCGGCACUACUGCUACAGCCUGUACGGCACCACACUGGAAC | |
| | AGCAGUACAACAAGCCCCUGUGCGACCUGCUGAUCCGGUGCAUCAACU | |
| | GCCAGAAACCUCUGUGUCCUGAGGAAAAGCAGAGACACCUGGACAAGA | |
| | AGCAGCGAUUUCACAACAUCAGAGGAAGAUGGACAGGCAGAUGCAUGA | |
| | GCUGCUGCAGAUCUAGCAGAACCAGAAGAGAGACACAGCUGUGA | |
| HPV 16 E7 | AUGCACGGCGACACCCCUACACUGCACGAGUACAUGCUGGACCUGCAGC | SEQ ID_No 3 |
| | CUGAAACCACCGACCUGUACUGCUACGAGCAGCUGAACGACUCUAGCG | |
| | AGGAAGAGGACGAGAUCGACGGCCCUGCCGGCCAGGCCGAGCCUGAUA | |
| | GAGCCCACUACAACAUCGUGACCUUCUGCUGCAAGUGCGACAGCACCCU | |
| | UAGACUGUGCGUGCAGAGCACACACGUGGACAUCAGAACCCUGGAAGA | |
| | UCUGCUGAUGGGCACCUUGGGCAUCGUGUGCCCCAUCUGCAGCCAGAA | |
| | GCCUUGA | |
| HPV16 E1 | AUGGCGGACCCCGCUGGCACCAACGGAGAAGAAGGCACCGGCUGCAAC | SEQ ID_No 4 |
| | GGCUGGUUCUACGUGGAAGCCGUGGUGGAAAAGAAGACCGGCGACGC | |
| | CAUCAGCGACGACGAGAAUGAGAACGAUAGCGACACCGGCGAGGAUCU | |
| | CGUCGAUUUCAUAGUCAAUGAUAACGAUUACCUGACGCAGGCCGAAAC | |
| | AGAAACGGCCCAUGCGCUGUUCACCGCACAAGAGGCGAAGCAGCAUAG | |
| | AGAUGCAGUGCAAGUAUUGAAAAGGAAGUAUUUGGUGAGCCCUCUGU | |
| | CUGACAUAAGCGGAUGUGUGGACAAUAAUAUAAGCCCACGCCUUAAGG | |
| | CAAUCUGUAUCGAGAAGCAGAGCAGGGCCGCGAAGAGGCGGCUGUUCG | |
| | AGAGCGAAGACAGCGGAUAUGGCAAUACCGAAGUGGAGACACAGCAAA | |
| | UGUUGCAAGUAGAAGGCCGCCACGAGACAGAAACCCCCUGUAGCCAGU | |
| | AUAGCGGUGGUAGCGGAGGUGGAUGUAGCCAGUACAGCAGCGGAAGC | |
| | GGCGGCGAAGGGGUUAGCGAGAGACAUACCAUCUGCCAAACUCCGCUG | |
| | ACGAACAUUUUGAAUGUGCUCAAGACGAGCAACGCCAAAGCUGCAAUG | |
| | CUUGCGAAGUUUAAAGAAUUGUACGGCGUUAGCUUUAGCGAAUUGGU | |
| | GAGACCCUUCAAGAGCAAUAAGAGCACUUGUUGUGAUUGGUGCAUCG | |
| | CUGCAUUUGGCCUUACACCGAGCAUUGCGGACAGCAUAAAGACUUUGC | |
| | UGCAACAGUACUGUCUGUACCUGCACAUUCAGAGCCUCGCAUGUAGCU | |
| | GGGGAAUGGUCGUGCUGCUCCUGGUGCGAUACAAGUGCGGAAAAAAU | |
| | AGAGAAACUAUAGAGAAGCUCCUCAGCAAGCUGCUCUGCGUUAGCCCA | |
| | AUGUGCAUGAUGAUUGAGCCCCCAAAGUUGCGAAGCACCGCCGCCGCA | |
| | CUUUACUGGUACAAGACUGGUAUAAGCAACAUCAGCGAAGUGUACGG | |
| | AGAUACACCCGAGUGGAUCCAGCGCCAAACCGUUCUCCAGCAUAGCUU | |
| | UAACGACUGUACGUUUGAAUUGAGCCAGAUGGUCCAGUGGGCGUAUG | |
| | ACAACGAUAUCGUUGACGACUCAGAAAUAGCUUACAAAUACGCACAGC | |
| | UCGCGGACACCAACAGCAACGCAAGCGCGUUUUUGAAGAGUAACAGCC | |
| | AAGCCAAGAUCGUUAAGGAUUGUGCGACUAUGUGUAGACACUAUAAG | |
| | CGCGCUGAGAAGAAACAAAUGUCAAUGAGCCAAUGGAUUAAAUACCGA | |
| | UGUGAUCGGGUUGAUGACGGUGGCGAUUGGAAGCAAAUUGUCAUGU | |
| | UUCUUAGAUAUCAGGGGGUUGAGUUCAUGAGCUUCUUGACAGCGCUC | |
| | AAGAGAUUUCUGCAAGGGAUCCCAAAGAAGAAUUGUAUACUCCUUUAC | |
| | GGAGCUGCCAAUACAGGGAAGAGUCUGUUCGGAAUGAGCCUGAUGAA | |
| | AUUCCUCCAAGGGAGCGUCAUCUGCUUUGUCAACAGCAAAAGCCACUU | |
| | UUGGCUUCAACCUCUCGCCGAUGCGAAAAUUGGUAUGCUCGAUGAUG | |
| | CUACCGUCCCAUGCUGGAACUACAUCGACGAUAACCUUAGAAAUGCAC | |
| | UUGACGGGAAUCUGGUGAGCAUGGAUGUUAAGCACAGACCUCUUGUG | |
| | CAGUUGAAAUGCCCUCCACUUCUCAUCACAUCAAAUAUUAACGCGGGG | |
| | ACGGACAGCCGAUGGCCCUAUCUGCACAAUCGGCUUGUGGUGUUCACG | |
| | UUCCCAAAUGAGUUCCCUUUCGACGAGAACGGAAAUCCAGUAUAUGAA | |
| | CUGAAUGACAAAAACUGGAAGAGUUUCUUCUCCCGGACCUGGAGCAGA | |
| | CUGAGCCUGCAUGAGGACGAGGAUAAAGAAAAUGACGGGGACAGCCUC | |
| | CCCACUUUCAAAUGCGUCUCUGGACAGAACACUAACACCUUGUAA | |

In one embodiment the vaccine comprises an mRNA polynucleotide comprising an open reading frame comprising a sequence selected from SEQ ID_No 2 or a sequence with at least 80% sequence identity or homology.

In one embodiment the vaccine comprises an mRNA polynucleotide comprising an open reading frame comprising a sequence selected from SEQ ID_No 2 or a sequence with at least 90% sequence identity or homology.

In one embodiment the vaccine comprises an mRNA polynucleotide comprising an open reading frame comprising a sequence selected from SEQ ID_No 2 or a sequence with at least 95% sequence identity or homology.

In one embodiment the vaccine comprises an mRNA polynucleotide comprising an open reading frame comprising a sequence selected from SEQ ID_No 2 or a sequence with at least 98% sequence identity or homology.

In one embodiment the vaccine comprises an mRNA polynucleotide comprising an open reading frame comprising a sequence selected from SEQ ID_No 2.

In one embodiment the vaccine comprises an mRNA polynucleotide comprising an open reading frame consisting essentially of a sequence selected from SEQ ID_No 2.

In one embodiment the vaccine comprises an mRNA polynucleotide comprising an open reading frame consisting of a sequence selected from SEQ ID_No 2.

In one embodiment the vaccine comprises an mRNA polynucleotide comprising an open reading frame comprising a sequence selected from SEQ ID_No 3 or a sequence with at least 80% sequence identity or homology.

In one embodiment the vaccine comprises an mRNA polynucleotide comprising an open reading frame comprising a sequence selected from SEQ ID_No 3 or a sequence with at least 90% sequence identity or homology.

In one embodiment the vaccine comprises an mRNA polynucleotide comprising an open reading frame comprising a sequence selected from SEQ ID_No 3 or a sequence with at least 95% sequence identity or homology.

In one embodiment the vaccine comprises an mRNA polynucleotide comprising an open reading frame comprising a sequence selected from SEQ ID_No 3 or a sequence with at least 98% sequence identity or homology.

In one embodiment the vaccine comprises an mRNA polynucleotide comprising an open reading frame comprising a sequence selected from SEQ ID_No 3.

In one embodiment the vaccine comprises an mRNA polynucleotide comprising an open reading frame consisting essentially of a sequence selected from SEQ ID_No 3.

In one embodiment the vaccine comprises an mRNA polynucleotide comprising an open reading frame consisting of a sequence selected from SEQ ID_No 3.

In one embodiment the vaccine comprises an mRNA polynucleotide comprising an open reading frame comprising a sequence selected from SEQ ID_No 4 or a sequence with at least 80% sequence identity or homology.

In one embodiment the vaccine comprises an mRNA polynucleotide comprising an open reading frame comprising a sequence selected from SEQ ID_No 4 or a sequence with at least 90% sequence identity or homology.

In one embodiment the vaccine comprises an mRNA polynucleotide comprising an open reading frame comprising a sequence selected from SEQ ID_No 4 or a sequence with at least 95% sequence identity or homology.

In one embodiment the vaccine comprises an mRNA polynucleotide comprising an open reading frame comprising a sequence selected from SEQ ID_No 4 or a sequence with at least 98% sequence identity or homology.

In one embodiment the vaccine comprises an mRNA polynucleotide comprising an open reading frame comprising a sequence selected from SEQ ID_No 4.

In one embodiment the vaccine comprises an mRNA polynucleotide comprising an open reading frame consisting essentially of a sequence selected from SEQ ID_No 4.

In one embodiment the vaccine comprises an mRNA polynucleotide comprising an open reading frame consisting of a sequence selected from SEQ ID_No 4.

### Encoding an Antigen

An antigen is a protein or a polypeptide from an infectious microorganism that binds to a specific antibody or T-cell receptor, triggering an immune response.

The antigens from an infectious microorganism encoded by the mRNA polynucleotide in the vaccines described herein can be “wild-type” i.e. naturally occurring antigens, or modified naturally occurring antigens. Non naturally occurring antigens that may be encoded by the mRNA polynucleotides in the vaccines herein are similar enough (e.g. 80% sequence identity or homology) to provoke the same immunogenic reaction as the equivalent wild-type antigen.

In one embodiment the vaccine comprises an mRNA polynucleotide comprising an open reading frame encoding an antigen from an infectious microorganism, or an immunogenic fragment thereof, wherein the antigen, or an immunogenic fragment thereof, has at least 80% sequence identity or homology with the wild-type antigen or a fragment thereof.

In one embodiment the vaccine comprises an mRNA polynucleotide comprising an open reading frame encoding an antigen from an infectious microorganism, or an immunogenic fragment thereof, wherein the antigen, or an immunogenic fragment thereof, has at least 90% sequence identity or homology with the wild-type antigen or a fragment thereof.

In one embodiment the vaccine comprises an mRNA polynucleotide comprising an open reading frame encoding an antigen from an infectious microorganism, or an immunogenic fragment thereof, wherein the antigen, or an immunogenic fragment thereof, has at least 95% sequence identity or homology with the wild-type antigen or a fragment thereof.

In one embodiment the vaccine comprises an mRNA polynucleotide comprising an open reading frame encoding an antigen from an infectious microorganism, or an immunogenic fragment thereof, wherein the antigen, or an immunogenic fragment thereof, has at least 98% sequence identity or homology with the wild-type antigen or a fragment thereof.

In one embodiment the vaccine comprises an mRNA polynucleotide comprising an open reading frame encoding an antigen from an infectious microorganism, or an immunogenic fragment thereof, wherein the antigen, or an immunogenic fragment thereof, is identical the wild-type antigen or a fragment thereof.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 80-700 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 80-180 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 600-700 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of about 649 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 649 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 140-170 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of about 158 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 158 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 90-110 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of about 98 amino acid residues.

In one embodiment the vaccine comprises an mRNA polynucleotide comprising an open reading frame encoding an antigen from an infectious microorganism or an immunogenic fragment thereof wherein the antigen or immunogenic fragment thereof is a polypeptide consisting of 98 amino acid residues.

In one embodiment the vaccine comprises an mRNA polynucleotide comprising an open reading frame encoding an HPV antigen or an immunogenic fragment thereof.

In one embodiment the vaccine comprises an mRNA polynucleotide comprising an open reading frame encoding an HPV antigen or an immunogenic fragment thereof forming part or all of the HPV viral capsid.

In one embodiment the vaccine comprises an mRNA polynucleotide comprising an open reading frame encoding an HPV antigen or an immunogenic fragment thereof responsible for binding the HPV to a cell being infected.

In one embodiment the vaccine comprises an mRNA polynucleotide comprising an open reading frame encoding an antigen from an infectious microorganism that interacts with retinoblastoma protein (pRb), or an immunogenic fragment thereof.

In one embodiment the vaccine comprises an mRNA polynucleotide comprising an open reading frame encoding an antigen from an infectious microorganism that interacts with p53, or an immunogenic fragment thereof.

In one embodiment the vaccine comprises an mRNA polynucleotide comprising an open reading frame encoding an antigen from an infectious microorganism that attaches to cell receptors, or an immunogenic fragment thereof.

In one embodiment the vaccine comprises an mRNA polynucleotide comprising an open reading frame encoding an antigen from an infectious microorganism that causes fusion of viral and cellular membranes, or an immunogenic fragment thereof.

In one embodiment the vaccine comprises an mRNA polynucleotide comprising an open reading frame encoding an antigen from an infectious microorganism that is responsible for binding of the virus to a cell being infected, or an immunogenic fragment thereof.

Table 2 describes sequences of antigens from an infectious microorganism that the mRNA polynucleotides of the vaccines described herein may encode for:

TABLE 2
Antigens

| Antigen | Sequence | SEQ ID_No |
|---|---|---|
| HPV 16 E6 | MHQKRTAMFQDPQERPRKLPQLCTELQTTIHDIILECVYCKQQLLRREVYDFAF | SEQ ID_No 5 |
| | RDLCIVYRDGNPYAVCDKCLKFYSKISEYRHYCYSLYGTTLEQQYNKPLCDLLIR | |
| | CINCQKPLCPEEKQRHLDKKQRFHNIRGRWTGRCMSCCRSSRTRRETQL | |
| HPV 16 E7 | MHGDTPTLHEYMLDLQPETTDLYCYEQLNDSSEEEDEIDGPAGQAEPDRAHY | SEQ ID_No 6 |
| | NIVTFCCKCDSTLRLCVQSTHVDIRTLEDLLMGTLGIVCPICSQKP | |
| HPV 16 E1 | MADPAGTNGEEGTGCNGWFYVEAVVEKKTGDAISDDENENDSDTGEDLVD | SEQ ID_No 7 |
| | FIVNDNDYLTQAETETAHALFTAQEAKQHRDAVQVLKRKYLVSPLSDISGCVD | |
| | NNISPRLKAICIEKQSRAAKRRLFESEDSGYGNTEVETQQMLQVEGRHETETPC | |
| | SQYSGGSGGGCSQYSSGSGGEGVSERHTICQTPLTNILNVLKTSNAKAAMLAKFK | |
| | ELYGVSFSELVRPFKSNKSTCCDWCIAAFGLTPSIADSIKTLLQQYCLYLHIQSL | |
| | ACSWGMVVLLLVRYKCGKNRETIEKLLSKLLCVSPMCMMIEPPKLRSTAAALY | |
| | WYKTGISNISEVYGDTPEWIQRQTVLQHSFNDCTFELSQMVQWAYDNDIVD | |
| | DSEIAYKYAQLADTNSNASAFLKSNSQAKIVKDCATMCRHYKRAEKKQMSMS | |
| | QWIKYRCDRVDDGGDWKQIVMFLRYQGVEFMSFLTALKRFLQGIPKKNCILL | |
| | YGAANTGKSLFGMSLMKFLQGSVICFVNSKSHFWLQPLADAKIGMLDDATVP | |
| | CWNYIDDNLRNALDGNLVSMDVKHRPLVQLKCPPLLITSNINAGTDSRWPYL | |
| | HNRLVVFTFPNEFPFDENGNPVYELNDKNWKSFFSRTWSRLSLHEDEDKEND | |
| | GDSLPTFKCVSGQNTNTL | |

In one embodiment the vaccine comprises an mRNA polynucleotide comprising an open reading frame encoding an antigen comprising a sequence selected from SEQ ID_No 5 or a sequence with at least 80% sequence identity or homology.

In one embodiment the vaccine comprises an mRNA polynucleotide comprising an open reading frame encoding an antigen comprising a sequence selected from SEQ ID_No 5 or a sequence with at least 90% sequence identity or homology.

In one embodiment the vaccine comprises an mRNA polynucleotide comprising an open reading frame encoding an antigen comprising a sequence selected from SEQ ID_No 5 or a sequence with at least 95% sequence identity or homology.

In one embodiment the vaccine comprises an mRNA polynucleotide comprising an open reading frame encoding an antigen comprising a sequence selected from SEQ ID_No 5 or a sequence with at least 98% sequence identity or homology.

In one embodiment the vaccine comprises an mRNA polynucleotide comprising an open reading frame encoding an antigen comprising a sequence selected from SEQ ID_No 5.

In one embodiment the vaccine comprises an mRNA polynucleotide comprising an open reading frame encoding an antigen consisting essentially of a sequence selected from SEQ ID_No 5.

In one embodiment the vaccine comprises an mRNA polynucleotide comprising an open reading frame encoding an antigen consisting of SEQ ID_No 5.

In one embodiment there is provided an mRNA polynucleotide comprising an open reading frame encoding an antigen comprising a sequence selected from SEQ ID_No 5.

In one embodiment the vaccine comprises an mRNA polynucleotide comprising an open reading frame encoding an antigen comprising a sequence selected from SEQ ID_No 6 or a sequence with at least 80% sequence identity or homology.

In one embodiment the vaccine comprises an mRNA polynucleotide comprising an open reading frame encoding an antigen comprising a sequence selected from SEQ ID_No 6 or a sequence with at least 90% sequence identity or homology.

In one embodiment the vaccine comprises an mRNA polynucleotide comprising an open reading frame encoding an antigen comprising a sequence selected from SEQ ID_No 6 or a sequence with at least 95% sequence identity or homology.

In one embodiment the vaccine comprises an mRNA polynucleotide comprising an open reading frame encoding an antigen comprising a sequence selected from SEQ ID_No 6 or a sequence with at least 98% sequence identity or homology.

In one embodiment the vaccine comprises an mRNA polynucleotide comprising an open reading frame encoding an antigen comprising a sequence selected from SEQ ID_No 6.

In one embodiment there is provided an mRNA polynucleotide comprising an open reading frame encoding an antigen comprising a sequence selected from SEQ ID_No 6.

In one embodiment the vaccine comprises an mRNA polynucleotide comprising an open reading frame encoding an antigen consisting essentially of a sequence selected from SEQ ID_No 6.

In one embodiment the vaccine comprises an mRNA polynucleotide comprising an open reading frame encoding an antigen consisting of SEQ ID_No 6.

In one embodiment the vaccine comprises an mRNA polynucleotide comprising an open reading frame encoding an antigen comprising a sequence selected from SEQ ID_No 7 or a sequence with at least 80% sequence identity or homology.

In one embodiment the vaccine comprises an mRNA polynucleotide comprising an open reading frame encoding an antigen comprising a sequence selected from SEQ ID_No 7 or a sequence with at least 90% sequence identity or homology.

In one embodiment the vaccine comprises an mRNA polynucleotide comprising an open reading frame encoding an antigen comprising a sequence selected from SEQ ID_No 7 or a sequence with at least 95% sequence identity or homology.

In one embodiment the vaccine comprises an mRNA polynucleotide comprising an open reading frame encoding an antigen comprising a sequence selected from SEQ ID_No 7 or a sequence with at least 98% sequence identity or homology.

In one embodiment the vaccine comprises an mRNA polynucleotide comprising an open reading frame encoding an antigen comprising a sequence selected from SEQ ID_No 7.

In one embodiment there is provided an mRNA polynucleotide comprising an open reading frame encoding an antigen comprising a sequence selected from SEQ ID_No 7.

In one embodiment the vaccine comprises an mRNA polynucleotide comprising an open reading frame encoding an antigen consisting essentially of a sequence selected from SEQ ID_No 7.

In one embodiment the vaccine comprises an mRNA polynucleotide comprising an open reading frame encoding an antigen consisting of SEQ ID_No 7.

Table 3 shows gene bank accession numbers for the sequences of certain HPV antigens from an infectious microorganism that the mRNA polynucleotides of the vaccines described herein may encode for:

TABLE 3
Gene bank accession numbers for certain HPV antigens

| | HPV | GenBank/UniProtKB/Swiss-Prot |
|---|---|---|
| HPV strain | protein | Accession |
| Human papilloma | L1 | QEE83786.1 |
| virus type 6 | L2 | QEE83785.1 |
| | E1 | QEE83780.1 |
| | E2 | QEE83781.1 |
| | E4 | QEE83782.1 |
| | E5A | QEE83783.1 |
| | E5B | QEE83784.1 |
| | E6 | QEE83778.1 |
| | E7 | QEE83779.1 |
| Human papilloma | L1 | P06417.1 |
| virus type 8 | L2 | P06419.1 |
| | E1 | P06420.1 |
| | E2 | P06422.1 |
| | E4 | P06425.1 |
| | E6 | P06428.1 |
| | E7 | P06430.1 |
| Human papilloma | L1 | QEE83895.1 |
| virus type 11 | L2 | QEE83894.1 |
| | E1 | QEE83889.1 |
| | E2 | QEE83890.1 |
| | E4 | QEE83891.1 |
| | E5A | QEE83892.1 |
| | E5B | QEE83893.1 |
| | E6 | QEE83887.1 |
| | E7 | QEE83888.1 |
| Human papilloma | L1 | AYV61481.1 |
| virus type 16 | L2 | AYV61480.1 |
| | E1 | AYV61476.1 |
| | E2 | AYV61477.1 |
| | E4 | AYV61478.1 |
| | E5 | AYV61479.1 |
| | E6 | AAL01351.1 |
| | E7 | NP_041326.1 |
| Human papilloma | L1 | ATL15246.1 |
| virus type 18 | L2 | ATL15245.1 |
| | E1 | AGU90874.1 |
| | E2 | ATL15234.1 |
| | E4 | ATL15243.1 |
| | E5 | ATL15244.1 |
| | E6 | ATL15239.1 |
| | E7 | ATL15240.1 |
| Human papilloma | L1 | ASL24648.1 |
| virus type 31 | L2 | CAD1807847.1 |
| | E1 | CAD1807839.1 |
| | E2 | CAD1807842.1 |
| | E4 | CAD1807843.1 |
| | E5 | CAD1814371.1 |
| | E6 | CAD1807836.1 |
| | E7 | CAD1807837.1 |
| Human papilloma | L1 | ASL24649.1 |
| virus type 33 | L2 | AEI61868.1 |
| | E1 | AEI61432.1 |
| | E2 | AEI61865.1 |
| | E4 | CAD1814538.1 |
| | E5 | CAD1814539.1 |
| | E6 | AFO63342.1 |
| | E7 | AFO63343.1 |
| Human papilloma | L1 | QJD33099.1 |
| virus type 35 | L2 | QJD38258.1 |
| | E1 | QJD33301.1 |
| | E2 | QJD36478.1 |
| | E4 | QJD38256.1 |
| | E5 | QJD38249.1 |
| | E6 | QJD38252.1 |
| | E7 | QJD38253.1 |
| Human papilloma | L1 | CAD1814503.1 |
| virus type 39 | L2 | CAD1814501.1 |
| | E1 | AGU90577.1 |
| | E2 | CAD1814494.1 |
| | E4 | CAD1814496.1 |
| | E5 | AGU90579.1 |
| | E6 | CAD1814490.1 |
| | E7 | CAD1814491.1 |
| Human papilloma | L1 | CAD1814430.1 |
| virus type 45 | L2 | CAD1814429.1 |
| | E1 | AGU90627.1 |
| | E2 | CAD1814428.1 |
| | E4 | CAD1807061.1 |
| | E5 | CAD1807062.1 |
| | E6 | CAD1814425.1 |
| | E7 | CAD1814426.1 |
| Human papilloma | L1 | AMK51256.1 |
| virus type 51 | L2 | AMK51255.1 |
| | E1 | AMK51253.1 |
| | E2 | AMK51252.1 |
| | E4 | AMK51254.1 |
| | E5 | P26553.1 |
| | E6 | P26554.1 |
| | E7 | P26558.1 |
| Human papilloma | L1 | CAD1814438.1 |
| virus type 52 | L2 | CAD1814437.1 |
| | E1 | CAD1814433.1 |
| | E2 | CAD1814434.1 |
| | E4 | CAD1814435.1 |
| | E5 | CAD1814436.1 |
| | E6 | CAD1814431.1 |
| | E7 | CAD1814432.1 |
| Human papilloma | L1 | CAD1814189.1 |
| virus type 56 | L2 | CAD1814186.1 |
| | E1 | CAD1814181.1 |
| | E2 | CAD1814183.1 |
| | E4 | CAD1814185.1 |
| | E6 | CAD1814586.1 |
| | E7 | CAD1814179.1 |
| Human papilloma | L1 | CAD1813847.1 |
| virus type 58 | L2 | CAD1813846.1 |
| | E1 | CAD1813842.1 |
| | E2 | CAD1813843.1 |
| | E4 | CAD1813844.1 |
| | E5 | CAD1813845.1 |
| | E6 | CAD1813840.1 |
| | E7 | CAD1813841.1 |
| Human papilloma | L1 | CAD1814219.1 |
| virus type 59 | L2 | CAD1814217.1 |
| | E1 | CAD1814209.1 |
| | E2 | CAD1814211.1 |
| | E4 | CAD1814213.1 |
| | E5 | CAD1814215.1 |
| | E6 | CAD1814206.1 |
| | E7 | CAD1814207.1 |
| Human papilloma | L1 | CAD1814518.1 |
| virus type 66 | L2 | CAD1814517.1 |
| | E1 | CAD1814510.1 |
| | E2 | CAD1814512.1 |
| | E4 | CAD1814515.1 |
| | E6 | CAD1814506.1 |
| | E7 | CAD1814507.1 |
| Human papilloma | L1 | CAD1807650.1 |
| virus type 68 | L2 | CAD1807649.1 |
| | E1 | CAD1807646.1 |
| | E2 | CAD1807647.1 |
| | E4 | CAD1807371.1 |
| | E5 | CAD1807648.1 |
| | E6 | CAD1807644.1 |
| | E7 | CAD1807645.1 |

In one embodiment the vaccine comprises an mRNA polynucleotide comprising an open reading frame encoding an antigen comprising a sequence selected from Table 3 or a sequence with at least 80% sequence identity or homology.

In one embodiment the vaccine comprises an mRNA polynucleotide comprising an open reading frame encoding an antigen comprising a sequence selected from Table 3 or a sequence with at least 90% sequence identity or homology.

In one embodiment the vaccine comprises an mRNA polynucleotide comprising an open reading frame encoding an antigen comprising a sequence selected from Table 3 or a sequence with at least 95% sequence identity or homology.

In one embodiment the vaccine comprises an mRNA polynucleotide comprising an open reading frame encoding an antigen comprising a sequence selected from Table 3 or a sequence with at least 98% sequence identity or homology.

In one embodiment the vaccine comprises an mRNA polynucleotide comprising an open reading frame encoding an antigen comprising a sequence selected from Table 3.

In one embodiment the vaccine comprises an mRNA polynucleotide comprising an open reading frame encoding an antigen consisting essentially of a sequence selected from Table 3.

In one embodiment the vaccine comprises an mRNA polynucleotide comprising an open reading frame encoding an antigen consisting of a sequence selected from Table 3.

### Immunogenic Fragment Thereof

An immunogenic fragment of an antigen (also known as an epitope, or antigenic determinant) is the fragment of the antigen that is recognized by the immune system, specifically by antibodies, B cells, or T cells, and induces an immune response. Immunogenic fragments that provoke a CD8+ response are typically 8-10 amino acids long.

### An Infectious Microorganism

Infectious microorganisms fall into 5 main categories-bacteria, parasites (for example protozoa), fungi, viruses and prions. These pathogenic microorganisms can be transmitted by animals, humans, insects or other agents causing diseases in the host organism.

In one embodiment the vaccine comprises an mRNA polynucleotide comprising an open reading frame encoding an antigen from a bacterium, parasite, fungus, virus or prion or an immunogenic fragment thereof.

In one embodiment the vaccine comprises an mRNA polynucleotide comprising an open reading frame encoding an antigen from a bacterium or an immunogenic fragment thereof.

In one embodiment the vaccine comprises an mRNA polynucleotide comprising an open reading frame encoding an antigen from a parasite or an immunogenic fragment thereof.

In one embodiment the vaccine comprises an mRNA polynucleotide comprising an open reading frame encoding an antigen from a fungus or an immunogenic fragment thereof.

In one embodiment the vaccine comprises an mRNA polynucleotide comprising an open reading frame encoding an antigen from a virus or an immunogenic fragment thereof.

In one embodiment the vaccine comprises an mRNA polynucleotide comprising an open reading frame encoding an antigen from a prion or an immunogenic fragment thereof.

### Multiple Component Vaccines

In one embodiment the vaccine comprises multiple, e.g. 2-10, mRNA polynucleotides comprising open reading frames each encoding a single antigen from an infectious microorganism or an immunogenic fragment thereof. The two or more mRNA polynucleotides may encode the same or different antigens, or sequences from different parts of the same antigen, additionally or alternatively from different strains of infectious microorganisms.

In one embodiment the vaccine comprises one mRNA polynucleotide comprising open reading frames encoding multiple, e.g. 2-10, antigens from an infectious microorganism or an immunogenic fragment thereof (e.g. as a fusion polypeptide). The open reading frames encoding multiple antigens from an infectious microorganism or an immunogenic fragment thereof may encode the same or different antigens, or sequences from different parts of the same antigen, additionally or alternatively from different strains of infectious microorganisms.

In one embodiment, the vaccines described herein are multivalent. A multivalent vaccine combines antigens from different strains of one pathogen to immunize against one disease.

In one embodiment the vaccine comprises a first mRNA polynucleotide comprising an open reading frame encoding an antigen from an infectious microorganism or an immunogenic fragment thereof and a second mRNA polynucleotide comprising an open reading frame encoding an antigen from an infectious microorganism or an immunogenic fragment thereof.

In one embodiment the vaccine comprises a first mRNA polynucleotide comprising an open reading frame encoding an antigen from an infectious microorganism or an immunogenic fragment thereof, and a second mRNA polynucleotide comprising an open reading frame encoding an antigen from an infectious microorganism or an immunogenic fragment thereof, and a third mRNA polynucleotide comprising an open reading frame encoding an antigen from an infectious microorganism or an immunogenic fragment thereof.

In one embodiment the vaccine comprises one mRNA polynucleotide comprising open reading frames encoding two antigens from an infectious microorganism or an immunogenic fragment thereof.

In one embodiment the vaccine comprises one mRNA polynucleotide comprising open reading frames encoding three antigens from an infectious microorganism or an immunogenic fragment thereof.

In one embodiment the vaccine comprises:
i) a first mRNA polynucleotide comprising an open reading frame encoding an antigen from an infectious microorganism or an immunogenic fragment thereof;
ii) a second mRNA polynucleotide comprising an open reading frame encoding an antigen from an infectious microorganism or an immunogenic fragment thereof; and
iii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an antigen from an infectious microorganism or an immunogenic fragment thereof;
ii) a second mRNA polynucleotide comprising an open reading frame encoding an antigen from an infectious microorganism or an immunogenic fragment thereof;
ii) a third mRNA polynucleotide comprising an open reading frame encoding an antigen from an infectious microorganism or an immunogenic fragment thereof; and
iv) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises two mRNA polynucleotides comprising an open reading frame each encoding an antigen from an infectious microorganism or an immunogenic fragment thereof wherein the two mRNA polynucleotides are present in about a 50:50% w/w ratio.

In one embodiment the vaccine comprises three mRNA polynucleotides comprising an open reading frame each encoding an antigen from an infectious microorganism or an immunogenic fragment thereof wherein the three mRNA polynucleotides are present in about equal % w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in a 50:50% w/w ratio.

In one embodiment the vaccine comprises three mRNA polynucleotides comprising an open reading frame each encoding an antigen from an infectious microorganism or an immunogenic fragment thereof wherein the three mRNA polynucleotides are present in equal % w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in about a 90:10% w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in a 90:10% w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in about a 75:25% w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in a 75:25% w/w ratio.

In one embodiment the vaccine comprises two mRNA polynucleotides comprising an open reading frame each encoding an antigen from an infectious microorganism or an immunogenic fragment thereof wherein the two mRNA polynucleotides are present in a 1:1 copy number ratio.

“Copy number” refers to the number of copies of the mRNA present. To determine copy numbers of mRNA, the molecular weight (MW) of one copy of mRNA is calculated by addition of the molecular weights of all constituent nucleotides in the entire nucleotide sequence, where:
A=329.2 g/mol
C=305.2 g/mol
G=345.2 g/mol
T=306.2 g/mol

Any proprietary sequences (non-ORF), for which the precise nucleotides are not known, are accounted for by using the average molecular weight of A/G/C/T=321.45 g/mol.

The copy numbers present in a known mass of mRNA (m) are then calculated as follows:

Copy Number=(m/MW)*NA
(where NA=Avogadro's Number=6.0221409×10\hat{ }23)

In one embodiment the vaccine comprises three mRNA polynucleotides comprising an open reading frame each encoding an antigen from an infectious microorganism or an immunogenic fragment thereof wherein the three mRNA polynucleotides are present in a 1:1:1 copy number ratio.

In one embodiment the vaccine comprises an mRNA polynucleotide comprising two or more open reading frames each encoding an antigen from an infectious microorganism or an immunogenic fragment thereof wherein the open reading frames are present in an equal copy number ratio.

In one embodiment the vaccine comprises two mRNA polynucleotides comprising an open reading frame each encoding an antigen from an infectious microorganism or an immunogenic fragment thereof wherein the two mRNA polynucleotides are present in about 1:1 copy number ratio.

In one embodiment the vaccine comprises three mRNA polynucleotides comprising an open reading frame each encoding an antigen from an infectious microorganism or an immunogenic fragment thereof wherein the three mRNA polynucleotides are present in about 1:1:1 copy number ratio.

In one embodiment the vaccine comprises an mRNA polynucleotide comprising two or more open reading frames each encoding an antigen from an infectious microorganism or an immunogenic fragment thereof wherein the open reading frames are present in about an equal copy number ratio.

In one embodiment the vaccine comprises two mRNA polynucleotides comprising an open reading frame each encoding an antigen from an infectious microorganism or an immunogenic fragment thereof wherein the two mRNA polynucleotides are present in a 3:1 copy number ratio.

In one embodiment the vaccine comprises two mRNA polynucleotides comprising an open reading frame each encoding an antigen from an infectious microorganism or an immunogenic fragment thereof wherein the two mRNA polynucleotides are present in about 3:1 copy number ratio.

In one embodiment the vaccine comprises two mRNA polynucleotides comprising an open reading frame each encoding an antigen from an infectious microorganism or an immunogenic fragment thereof wherein the two mRNA polynucleotides are present in a 9:1 copy number ratio.

In one embodiment the vaccine comprises two mRNA polynucleotides comprising an open reading frame each encoding an antigen from an infectious microorganism or an immunogenic fragment thereof wherein the two mRNA polynucleotides are present in about 9:1 copy number ratio.

In one embodiment the vaccine comprises three mRNA polynucleotides comprising open reading frames each encoding an HPV antigen selected from E1, E6 and E7 or an immunogenic fragment thereof wherein the three mRNA polynucleotides are present in about 1 (E1):3 (E6):4 (E7) copy number ratio.

In one embodiment the vaccine comprises three mRNA polynucleotides comprising open reading frames each encoding an HPV antigen selected from E1, E6 and E7 or an immunogenic fragment thereof wherein the three mRNA polynucleotides are present in 1 (E1):3 (E6):4 (E7) copy number ratio. In one embodiment the vaccine comprises three mRNA polynucleotides comprising open reading frames each encoding an HPV antigen selected from E1, E6 and E7 or an immunogenic fragment thereof wherein the three mRNA polynucleotides are present in about 1 (E1):3.2 (E6):4.2 (E7) copy number ratio.

In one embodiment the vaccine comprises three mRNA polynucleotides comprising open reading frames each encoding an HPV antigen selected from E1, E6 and E7 or an immunogenic fragment thereof wherein the three mRNA polynucleotides are present in 1 (E1):3.2 (E6):4.2 (E7) copy number ratio.

In one embodiment the vaccine comprises two mRNA polynucleotides comprising open reading frames each encoding an HPV antigen selected from E1, E2, E4, E5, E6, E7, L1, or L2 or an immunogenic fragment thereof.

In one embodiment the vaccine comprises three mRNA polynucleotides comprising open reading frames each encoding an HPV antigen selected from E1, E2, E4, E5, E6, E7, L1, or L2 or an immunogenic fragment thereof.

In one embodiment the vaccine comprises two mRNA polynucleotides comprising open reading frames each encoding an HPV antigen selected from E1, E2, E4, E5, E6 or E7 or an immunogenic fragment thereof.

In one embodiment the vaccine comprises three mRNA polynucleotides comprising open reading frames each encoding an HPV antigen selected from E1, E2, E4, E5, E6 or E7 or an immunogenic fragment thereof.

In one embodiment the vaccine comprises two mRNA polynucleotides comprising open reading frames one encoding an E6 HPV antigen or an immunogenic fragment thereof, and the other encoding an E7 HPV antigen or an immunogenic fragment thereof.

In one embodiment the vaccine comprises three mRNA polynucleotides comprising open reading frames one encoding an E6 HPV antigen or an immunogenic fragment thereof, one encoding an E7 HPV antigen or an immunogenic fragment thereof, and one encoding an E1 HPV antigen or an immunogenic fragment thereof.

In one embodiment the vaccine comprises two mRNA polynucleotides comprising open reading frames one encoding an L1 HPV antigen or an immunogenic fragment thereof, and the other encoding an L2 HPV antigen or an immunogenic fragment thereof.

In one embodiment the vaccine comprises two mRNA polynucleotides comprising an open reading frame each encoding a high-risk HPV antigen or an immunogenic fragment thereof.

In one embodiment the vaccine comprises three mRNA polynucleotides comprising an open reading frame each encoding a high-risk HPV antigen or an immunogenic fragment thereof.

In one embodiment the vaccine comprises two mRNA polynucleotides comprising an open reading frame each encoding an HPV 16, 18, 31, 33, 35, 39, 45, 51, 52, 56, 58, 59, 66, and/or 68 antigen or an immunogenic fragment thereof.

In one embodiment the vaccine comprises three mRNA polynucleotides comprising an open reading frame each encoding an HPV 16, 18, 31, 33, 35, 39, 45, 51, 52, 56, 58, 59, 66, and/or 68 antigen or an immunogenic fragment thereof.

In one embodiment the vaccine comprises two mRNA polynucleotides comprising an open reading frame each encoding an HPV 6, 8, 11, 16 and/or 18 antigen or an immunogenic fragment thereof.

In one embodiment the vaccine comprises three mRNA polynucleotides comprising an open reading frame each encoding an HPV 6, 8, 11, 16 and/or 18 antigen or an immunogenic fragment thereof.

In one embodiment the vaccine comprises two mRNA polynucleotides comprising an open reading frame each encoding an HPV 16 and/or 18 antigen or an immunogenic fragment thereof.

In one embodiment the vaccine comprises three mRNA polynucleotides comprising an open reading frame each encoding an HPV 16 and/or 18 antigen or an immunogenic fragment thereof.

In one embodiment the vaccine comprises two mRNA polynucleotides comprising an open reading frame each encoding a low-risk HPV antigen or an immunogenic fragment thereof.

In one embodiment the vaccine comprises three mRNA polynucleotides comprising an open reading frame each encoding a low-risk HPV antigen or an immunogenic fragment thereof.

In one embodiment the vaccine comprises two mRNA polynucleotides comprising an open reading frame each encoding an HPV 6 and/or 11 antigen or an immunogenic fragment thereof. In one embodiment the vaccine comprises three mRNA polynucleotides comprising an open reading frame each encoding an HPV 6 and/or 11 antigen or an immunogenic fragment thereof.

In one embodiment the vaccine comprises two mRNA polynucleotides comprising an open reading frame one encoding a high-risk HPV antigen or an immunogenic fragment thereof, and the other encoding a low-risk HPV antigen or an immunogenic fragment thereof.

In one embodiment the vaccine comprises three mRNA polynucleotides comprising an open reading frame at least one encoding a high-risk HPV antigen or an immunogenic fragment thereof, and at least one encoding a low-risk HPV antigen or an immunogenic fragment thereof.

In one embodiment the vaccine comprises two mRNA polynucleotides comprising open reading frames one encoding an E6 HPV 16 antigen or an immunogenic fragment thereof, and the other encoding an E7 HPV 16 antigen or an immunogenic fragment thereof.

In one embodiment the vaccine comprises three mRNA polynucleotides comprising open reading frames one encoding an E6 HPV 16 antigen or an immunogenic fragment thereof, one encoding an E7 HPV 16 antigen or an immunogenic fragment thereof, and one encoding an E1 HPV 16 antigen or an immunogenic fragment thereof.

In one embodiment the vaccine comprises two mRNA polynucleotides comprising open reading frames one encoding an E6 HPV 18 antigen or an immunogenic fragment thereof, and the other encoding an E7 HPV 18 antigen or an immunogenic fragment thereof.

In one embodiment the vaccine comprises three mRNA polynucleotides comprising open reading frames one encoding an E6 HPV 18 antigen or an immunogenic fragment thereof, one encoding an E7 HPV 18 antigen or an immunogenic fragment thereof, and one an E1 HPV 18 antigen or an immunogenic fragment thereof.

In one embodiment the vaccine comprises two mRNA polynucleotides comprising open reading frames one encoding an E6 HPV 16 antigen or an immunogenic fragment thereof, and the other encoding an E7 HPV 18 antigen or an immunogenic fragment thereof.

In one embodiment the vaccine comprises three mRNA polynucleotides comprising open reading frames one encoding an E6 HPV 16 antigen or an immunogenic fragment thereof, one encoding an E7 HPV 18 antigen or an immunogenic fragment thereof, and one encoding an E1 HPV 18 antigen or an immunogenic fragment thereof.

In one embodiment the vaccine comprises two mRNA polynucleotides comprising open reading frames one encoding an E6 HPV 18 antigen or an immunogenic fragment thereof, and the other encoding an E7 HPV 16 antigen or an immunogenic fragment thereof.

In one embodiment the vaccine comprises two mRNA polynucleotides comprising open reading frames one encoding an E6 HPV antigen or an immunogenic fragment thereof, and the other encoding an E7 HPV antigen or an immunogenic fragment thereof wherein the two mRNA polynucleotides are present in about a 50:50% w/w ratio.

In one embodiment the vaccine comprises three mRNA polynucleotides comprising open reading frames one encoding an E6 HPV antigen or an immunogenic fragment thereof, one encoding an E7 HPV antigen or an immunogenic fragment thereof, and one encoding an E1 HPV antigen or an immunogenic fragment thereof wherein the three mRNA polynucleotides are present in about equal % w/w ratio.

In one embodiment the vaccine comprises two mRNA polynucleotides comprising open reading frames one encoding an E6 HPV antigen or an immunogenic fragment thereof, and the other encoding an E7 HPV antigen or an immunogenic fragment thereof wherein the two mRNA polynucleotides are present in a 50:50% w/w ratio.

In one embodiment the vaccine comprises three mRNA polynucleotides comprising open reading frames one encoding an E6 HPV antigen or an immunogenic fragment thereof, one encoding an E7 HPV antigen or an immunogenic fragment thereof, and one encoding an E1 HPV antigen or an immunogenic fragment thereof wherein the three mRNA polynucleotides are present in an equal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology.

In one embodiment the vaccine comprises an mRNA polynucleotide comprising an open reading frame comprising a sequence selected from SEQ ID_No 2 or a sequence with at least 80% sequence identity or homology, and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology wherein the two mRNA polynucleotides are present in about a 50:50% w/w ratio.

In one embodiment the vaccine comprises an mRNA polynucleotide comprising an open reading frame comprising a sequence selected from SEQ ID_No 2 or a sequence with at least 80% sequence identity or homology, and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in about equal % w/w ratio.

In one embodiment the vaccine comprises an mRNA polynucleotide comprising an open reading frame comprising a sequence selected from SEQ ID_No 2 or a sequence with at least 80% sequence identity or homology and an mRNA polynucleotide comprising an open reading frame comprising a sequence selected from SEQ ID_No 3 or a sequence with at least 80% sequence identity or homology wherein the two mRNA polynucleotides are present in a 50:50% w/w ratio.

In one embodiment the vaccine comprises an mRNA polynucleotide comprising an open reading frame comprising a sequence selected from SEQ ID_No 2 or a sequence with at least 80% sequence identity or homology, and an mRNA polynucleotide comprising an open reading frame comprising a sequence selected from SEQ ID_No 3 or a sequence with at least 80% sequence identity or homology, and an mRNA polynucleotide comprising an open reading frame comprising a sequence selected from SEQ ID_No 4 or a sequence with at least 80% sequence identity or homology wherein the three mRNA polynucleotides are present in a equal % w/w ratio.

In one embodiment the vaccine comprises an mRNA polynucleotide comprising an open reading frame comprising a sequence selected from SEQ ID_No 2 and an mRNA polynucleotide comprising an open reading frame comprising a sequence selected from SEQ ID_No 3 wherein the two mRNA polynucleotides are present in a about a 50:50% w/w ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4 wherein the three mRNA polynucleotides are present in a about a equal % w/w ratio.

In one embodiment the vaccine comprises an mRNA polynucleotide comprising an open reading frame comprising a sequence selected from SEQ ID_No 2 and an mRNA polynucleotide comprising an open reading frame comprising a sequence selected from SEQ ID_No 3 wherein the two mRNA polynucleotides are present in a 50:50% w/w ratio.

In one embodiment the vaccine comprises an mRNA polynucleotide comprising an open reading frame comprising a sequence selected from SEQ ID_No 2, and an mRNA polynucleotide comprising an open reading frame comprising a sequence selected from SEQ ID_No 3, and an mRNA polynucleotide comprising an open reading frame comprising a sequence selected from SEQ ID_No 4 wherein the three mRNA polynucleotides are present in equal % w/w ratio.

In one embodiment the vaccine comprises two mRNA polynucleotides comprising open reading frames one encoding an E6 HPV antigen selected from SEQ ID_No 5 or a sequence with at least 80% sequence identity or homology, and the other encoding an E7 HPV antigen selected from SEQ ID_No 6 or a sequence with at least 80% sequence identity or homology wherein the two mRNA polynucleotides are present in a 50:50% w/w ratio.

In one embodiment the vaccine comprises three mRNA polynucleotides comprising open reading frames one encoding an E6 HPV antigen selected from SEQ ID_No 5 or a sequence with at least 80% sequence identity or homology, one encoding an E7 HPV antigen selected from SEQ ID_No 6 or a sequence with at least 80% sequence identity or homology, and one encoding an E1 HPV antigen selected from SEQ ID_No 7 or a sequence with at least 80% sequence identity or homology wherein the three mRNA polynucleotides are present in a equal % w/w ratio.

In one embodiment the vaccine comprises two mRNA polynucleotides comprising open reading frames one encoding an E6 HPV antigen selected from SEQ ID_No 5, and the other encoding an E7 HPV antigen selected from SEQ ID_No 6 wherein the two mRNA polynucleotides are present in a 50:50% w/w ratio.

In one embodiment the vaccine comprises three mRNA polynucleotides comprising open reading frames one encoding an E6 HPV antigen selected from SEQ ID_No 5, one encoding an E7 HPV antigen selected from SEQ ID_No 6, and one encoding an E1 HPV antigen selected from SEQ ID_No 7 wherein the three mRNA polynucleotides are present in equal % w/w ratio.

In one embodiment the vaccine comprises two mRNA polynucleotides comprising open reading frames one encoding an E6 HPV antigen or an immunogenic fragment thereof, and the other encoding an E7 HPV antigen or an immunogenic fragment thereof wherein the two mRNA polynucleotides are present in about a 50:50% w/w ratio.

In one embodiment the vaccine comprises three mRNA polynucleotides comprising open reading frames one encoding an E6 HPV antigen or an immunogenic fragment thereof, one encoding an E7 HPV antigen or an immunogenic fragment thereof, and one encoding an E1 HPV antigen or an immunogenic fragment thereof, wherein the three mRNA polynucleotides are present in about equal % w/w ratio.

### Immunoadjuvants

In one embodiment the vaccine further comprises an immunoadjuvant. An immunoadjuvant is a substance that acts to accelerate, prolong, or enhance an antigen-specific immune response when used in combination with specific vaccine antigens.

Suitable immunoadjuvants include inorganic compounds, for example potassium alum KAI(SO₄)₂, aluminium hydroxide, aluminium phosphate, and calcium phosphate hydroxide; oils, for example paraffin oil, propolis, and peanut oil; and cytokines, for example IL-1 (interleukin-1), IL-2, IL-7, IL-12 and IL-15. Other suitable immunoadjuvants include granulocyte-macrophage colony-stimulating factor (GM-CSF).

In one embodiment the vaccine further comprises an mRNA polynucleotide comprising an open reading frame encoding an immunoadjuvant. In one embodiment the vaccine comprises an mRNA polynucleotide comprising an open reading frame encoding an antigen from an infectious microorganism or an immunogenic fragment thereof, and an open reading frame encoding an immunoadjuvant.

In one embodiment the vaccine comprises:
i) a first mRNA polynucleotide comprising an open reading frame comprising a sequence selected from SEQ ID_No 2;
ii) a second mRNA polynucleotide comprising an open reading frame comprising a sequence selected from SEQ ID_No 3;
iii) a third mRNA polynucleotide comprising an open reading frame comprising a sequence selected from SEQ ID_No 8; and
iv) an amphipathic cell penetrating peptide comprising the amino acid sequence WEARLARALARALARHLARALARALRACEA (SEQ ID_No 1).

In one embodiment the vaccine comprises:
i) a first mRNA polynucleotide comprising an open reading frame comprising a sequence selected from SEQ ID_No 2;
ii) a second mRNA polynucleotide comprising an open reading frame comprising a sequence selected from SEQ ID_No 3;
iii) a third mRNA polynucleotide comprising an open reading frame comprising a sequence selected from SEQ ID_No 4;
iv) a fourth mRNA polynucleotide comprising an open reading frame comprising a sequence selected from SEQ ID_No 8; and
v) an amphipathic cell penetrating peptide comprising the amino acid sequence WEARLARALARALARHLARALARALRACEA (SEQ ID_No 1).

In one embodiment the vaccine comprises:
i) a first mRNA polynucleotide comprising an open reading frame encoding an antigen from an infectious microorganism or an immunogenic fragment thereof;
ii) a second mRNA polynucleotide comprising an open reading frame encoding an antigen from an infectious microorganism or an immunogenic fragment thereof;
iii) a third mRNA polynucleotide comprising an open reading frame encoding an immunoadjuvant; and
iv) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an antigen from an infectious microorganism or an immunogenic fragment thereof;
ii) a second mRNA polynucleotide comprising an open reading frame encoding an antigen from an infectious microorganism or an immunogenic fragment thereof;
iii) a third mRNA polynucleotide comprising an open reading frame encoding an antigen from an infectious microorganism or an immunogenic fragment thereof;
iv) a fourth mRNA polynucleotide comprising an open reading frame encoding an immunoadjuvant; and
v) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine further comprises an mRNA polynucleotide comprising an open reading frame encoding a cytokine.

In one embodiment the vaccine further comprises an mRNA polynucleotide comprising an open reading frame encoding IL-1, IL-2, IL-7, IL-12 or IL-15.

In one embodiment the vaccine further comprises an mRNA polynucleotide comprising an open reading frame encoding IL-15.

In one embodiment the vaccine further comprises an mRNA polynucleotide comprising an open reading frame encoding human IL-15.

In one embodiment the vaccine further comprises an mRNA polynucleotide comprising an open reading frame encoding granulocyte-macrophage colony-stimulating factor.

In one embodiment the vaccine further comprises an mRNA polynucleotide comprising an open reading frame encoding human granulocyte-macrophage colony-stimulating factor.

In one embodiment the vaccine comprises:
i) a first mRNA polynucleotide comprising an open reading frame encoding an HPV E6 antigen or an immunogenic fragment thereof;
ii) a second mRNA polynucleotide comprising an open reading frame encoding an HPV E7 antigen or an immunogenic fragment thereof;
iii) a third mRNA polynucleotide comprising an open reading frame encoding human IL-15; and
iv) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an HPV E6 antigen or an immunogenic fragment thereof;
ii) a second mRNA polynucleotide comprising an open reading frame encoding an HPV E7 antigen or an immunogenic fragment thereof;
iii) a third mRNA polynucleotide comprising an open reading frame encoding an HPV E1 antigen or an immunogenic fragment thereof;
iv) a fourth mRNA polynucleotide comprising an open reading frame encoding human IL-15; and
v) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an HPV 16 E6 antigen or an immunogenic fragment thereof;
ii) a second mRNA polynucleotide comprising an open reading frame encoding an HPV 16 E7 antigen or an immunogenic fragment thereof;
iii) a third mRNA polynucleotide comprising an open reading frame encoding human IL-15; and
iv) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an HPV 16 E6 antigen or an immunogenic fragment thereof;
ii) a second mRNA polynucleotide comprising an open reading frame encoding an HPV 16 E7 antigen or an immunogenic fragment thereof;
iii) a third mRNA polynucleotide comprising an open reading frame encoding an HPV 16 E1 antigen or an immunogenic fragment thereof;
iv) a fourth mRNA polynucleotide comprising an open reading frame encoding human IL-15; and
v) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises:
i) a first mRNA polynucleotide comprising an open reading frame encoding an antigen selected from SEQ ID_No 5;
ii) a second mRNA polynucleotide comprising an open reading frame encoding an antigen selected from SEQ ID_No 6;
iii) a third mRNA polynucleotide comprising an open reading frame encoding an immunoadjuvant selected from SEQ ID_No 10; and
iv) an amphipathic cell penetrating peptide comprising the amino acid sequence WEARLARALARALARHLARALARALRACEA (SEQ ID_No 1).

In one embodiment the vaccine comprises:
i) a first mRNA polynucleotide comprising an open reading frame encoding an antigen selected from SEQ ID_No 5;
ii) a second mRNA polynucleotide comprising an open reading frame encoding an antigen selected from SEQ ID_No 6;
iii) a third mRNA polynucleotide comprising an open reading frame encoding an antigen selected from SEQ ID_No 7;
iv) a fourth mRNA polynucleotide comprising an open reading frame encoding an immunoadjuvant selected from SEQ ID_No 10; and
v) an amphipathic cell penetrating peptide comprising the amino acid sequence WEARLARALARALARHLARALARALRACEA (SEQ ID_No 1).

In one embodiment the vaccine further comprises up to 20% w/w (total mRNA) of an mRNA polynucleotide comprising an open reading frame encoding an immunoadjuvant. 20% w/w (total mRNA) means that the mRNA encoding the immunoadjuvant makes up 20% by weight of the total amount of mRNA in the vaccine.

In one embodiment the vaccine further comprises up to 20% w/w (total mRNA) of an mRNA polynucleotide comprising an open reading frame encoding human IL-15.

In one embodiment the vaccine further comprises 5-20% w/w (total mRNA) of an mRNA polynucleotide comprising an open reading frame encoding an immunoadjuvant.

In one embodiment the vaccine further comprises 5-20% w/w (total mRNA) of an mRNA polynucleotide comprising an open reading frame encoding human IL-15.

In one embodiment the vaccine further comprises 5% w/w (total mRNA) of an mRNA polynucleotide comprising an open reading frame encoding an immunoadjuvant.

In one embodiment the vaccine further comprises about 5% w/w (total mRNA) of an mRNA polynucleotide comprising an open reading frame encoding human IL-15.

In one embodiment the vaccine further comprises 10% w/w (total mRNA) of an mRNA polynucleotide comprising an open reading frame encoding an immunoadjuvant.

In one embodiment the vaccine further comprises about 10% w/w (total mRNA) of an mRNA polynucleotide comprising an open reading frame encoding human IL-15.

In one embodiment the vaccine further comprises 15% w/w (total mRNA) of an mRNA polynucleotide comprising an open reading frame encoding an immunoadjuvant.

In one embodiment the vaccine further comprises about 15% w/w (total mRNA) of an mRNA polynucleotide comprising an open reading frame encoding human IL-15.

In one embodiment the vaccine further comprises 20% w/w (total mRNA) of an mRNA polynucleotide comprising an open reading frame encoding an immunoadjuvant.

In one embodiment the vaccine further comprises about 20% w/w (total mRNA) of an mRNA polynucleotide comprising an open reading frame encoding human IL-15.

In one embodiment the vaccine further comprises up to 20% w/w (total mRNA) of an mRNA polynucleotide comprising an open reading frame encoding GM-CSF.

In one embodiment the vaccine further comprises 5-20% w/w (total mRNA) of an mRNA polynucleotide comprising an open reading frame encoding GM-CSF.

In one embodiment the vaccine further comprises about 5% w/w (total mRNA) of an mRNA polynucleotide comprising an open reading frame encoding GM-CSF.

In one embodiment the vaccine further comprises about 10% w/w (total mRNA) of an mRNA polynucleotide comprising an open reading frame encoding GM-CSF.

In one embodiment the vaccine further comprises about 15% w/w (total mRNA) of an mRNA polynucleotide comprising an open reading frame encoding GM-CSF.

In one embodiment the vaccine further comprises about 20% w/w (total mRNA) of an mRNA polynucleotide comprising an open reading frame encoding GM-CSF.

Table 4 describes certain mRNA open reading frame sequences encoding immunoadjuvants that may be incorporated mRNA polynucleotides and added to the vaccines described herein.

TABLE 4
mRNA polynucleotides encoding immunoadjuvants

| | mRNA Open Reading Frame Sequence | |
|---|---|---|
| Immunoadjuvant | (all uridines “U” are N1-methylpseudouridine nucleotides) | SEQ ID_No |
| hIL-15 (human | AUGAGAAUCAGCAAGCCGCACCUGAGAAGCAUCAGCAUUCAGU | SEQ ID_No 8 |
| interleukin 15) | GCUACCUCUGCCUGCUGCUCAACAGCCACUUCCUGACCGAAGC | |
| | CGGAAUCCACGUAUUCAUCCUGGGAUGCUUCAGCGCCGGCCUC | |
| | CCCAAAACAGAGGCAAACUGGGUAAACGUAAUAAGCGACCUCA | |
| | AAAAGAUCGAAGACCUGAUCCAGAGCAUGCACAUCGACGCAAC | |
| | CCUGUACACCGAAAGCGACGUACACCCAAGCUGCAAAGUAACC | |
| | GCGAUGAAAUGCUUCCUGCUGGAGCUGCAAGUAAUCAGCCUG | |
| | GAGAGCGGCGACGCCAGCAUCCACGACACCGUGGAAAACCUGA | |
| | UUAUCUUGGCAAACAACAGCCUGAGCAGCAACGGAAACGUGAC | |
| | AGAGAGCGGAUGCAAAGAGUGCGAGGAGCUGGAGGAAAAGAA | |
| | CAUCAAAGAAUUCCUCCAAAGCUUCGUGCACAUAGUGCAGAUG | |
| | UUCAUAAACACAAGCUGA | |
| hGM-CSF | AUGUGGCUGCAAAGCCUUCUGCUCUUGGGGACUGUGGCCUGC | SEQ ID_No 9 |
| | UCAAUUUCUGCACCUGCCCGAUCGCCCAGUCCAAGCACGCAGC | |
| | CCUGGGAGCAUGUGAAUGCCAUCCAGGAGGCGCGGAGGUUAC | |
| | UGAACCUGAGUAGAGACACUGCUGCUGAGAUGAAUGAAACAG | |
| | UAGAAGUGAUAUCAGAAAUGUUUGAUCUCCAGGAGCCUACCU | |
| | GCCUACAGACACGCCUGGAGCUGUAUAAACAGGGCCUGAGGG | |
| | GCUCCCUCACCAAGCUCAAAGGACCCUUGACAAUGAUGGCCAG | |
| | CCACUACAAGCAACACUGUCCUCCAACCCCGGAAACUUCCUGU | |
| | GCAACCCAGAUUAUCACAUUUGAAUCCUUCAAAGAGAACCUGA | |
| | AGGAUUUCCUACUUGUUAUCCCCUUCGACUGCUGGGAGCCAG | |
| | UCCAGGAGUGA | |

In one embodiment the vaccine further comprises an mRNA polynucleotide comprising an open reading frame encoding an immunoadjuvant comprising a sequence selected from SEQ ID_No 8 or a sequence with at least 80% sequence identity or homology.

In one embodiment the vaccine further comprises an mRNA polynucleotide comprising an open reading frame encoding an immunoadjuvant comprising a sequence selected from SEQ ID_No 8 or a sequence with at least 90% sequence identity or homology.

In one embodiment the vaccine further comprises an mRNA polynucleotide comprising an open reading frame encoding an immunoadjuvant comprising a sequence selected from SEQ ID_No 8 or a sequence with at least 95% sequence identity or homology.

In one embodiment the vaccine further comprises an mRNA polynucleotide comprising an open reading frame encoding an immunoadjuvant comprising a sequence selected from SEQ ID_No 8 or a sequence with at least 98% sequence identity or homology.

In one embodiment the vaccine further comprises an mRNA polynucleotide comprising an open reading frame encoding an immunoadjuvant comprising a sequence selected from SEQ ID_No 8.

In one embodiment there is provided an mRNA polynucleotide comprising an open reading frame encoding an immunoadjuvant comprising a sequence selected from SEQ ID_No 8.

In one embodiment the vaccine further comprises an mRNA polynucleotide comprising an open reading frame encoding an immunoadjuvant comprising a sequence selected from SEQ ID_No 9 or a sequence with at least 80% sequence identity or homology.

In one embodiment the vaccine further comprises an mRNA polynucleotide comprising an open reading frame encoding an immunoadjuvant comprising a sequence selected from SEQ ID_No 9 or a sequence with at least 90% sequence identity or homology.

In one embodiment the vaccine further comprises an mRNA polynucleotide comprising an open reading frame encoding an immunoadjuvant comprising a sequence selected from SEQ ID_No 9 or a sequence with at least 95% sequence identity or homology.

In one embodiment the vaccine further comprises an mRNA polynucleotide comprising an open reading frame encoding an immunoadjuvant comprising a sequence selected from SEQ ID_No 9 or a sequence with at least 98% sequence identity or homology.

In one embodiment the vaccine further comprises an mRNA polynucleotide comprising an open reading frame encoding an immunoadjuvant comprising a sequence selected from SEQ ID_No 9.

In one embodiment there is provided an mRNA polynucleotide comprising an open reading frame encoding an immunoadjuvant comprising a sequence selected from SEQ ID_No 9.

Table 5 describes sequences of immunoadjuvant that the mRNA polynucleotides of the vaccines described herein may encode for:

TABLE 5
Immunoadjuvant

| Immunoadjuvant | Sequence | SEQ ID_No |
|---|---|---|
| Human IL-15 | MRISKPHLRSISIQCYLCLLLNSHFLTEAGIHVFILGCFSAGLPKTEANW | SEQ ID_ |
| | VNVISDLKKIEDLIQSMHIDATLYTESDVHPSCKVTAMKCFLLELQVIS | No 10 |
| | LESGDASIHDTVENLIILANNSLSSNGNVTESGCKECEELEEKNIKEFLQ | |
| | SFVHIVQMFINTS | |
| Human GM-CSF | MWLQSLLLLGTVACSISAPARSPSPSTQPWEHVNAIQEARRLLNLSR | SEQ ID_ |
| | DTAAEMNETVEVISEMFDLQEPTCLQTRLELYKQGLRGSLTKLKGPLT | No 11 |
| | MMASHYKQHCPPTPETSCATQIITFESFKENLKDFLLVIPFDCWE | |
| | PVQE | |

In one embodiment the vaccine further comprises an mRNA polynucleotide comprising an open reading frame encoding an immunoadjuvant selected from SEQ ID_No 10 or a sequence with at least 80% sequence identity or homology.

In one embodiment the vaccine further comprises an mRNA polynucleotide comprising an open reading frame encoding an immunoadjuvant selected from SEQ ID_No 10 or a sequence with at least 90% sequence identity or homology.

In one embodiment the vaccine further comprises an mRNA polynucleotide comprising an open reading frame encoding an immunoadjuvant selected from SEQ ID_No 10 or a sequence with at least 95% sequence identity or homology.

In one embodiment the vaccine further comprises an mRNA polynucleotide comprising an open reading frame encoding an immunoadjuvant selected from SEQ ID_No 10 or a sequence with at least 98% sequence identity or homology.

In one embodiment the vaccine further comprises an mRNA polynucleotide comprising an open reading frame encoding an immunoadjuvant selected from SEQ ID_No 10.

In one embodiment the vaccine further comprises an mRNA polynucleotide comprising an open reading frame encoding an immunoadjuvant selected from SEQ ID_No 11 or a sequence with at least 80% sequence identity or homology.

In one embodiment the vaccine further comprises an mRNA polynucleotide comprising an open reading frame encoding an immunoadjuvant selected from SEQ ID_No 11 or a sequence with at least 90% sequence identity or homology.

In one embodiment the vaccine further comprises an mRNA polynucleotide comprising an open reading frame encoding an immunoadjuvant selected from SEQ ID_No 11 or a sequence with at least 95% sequence identity or homology.

In one embodiment the vaccine further comprises an mRNA polynucleotide comprising an open reading frame encoding an immunoadjuvant selected from SEQ ID_No 11 or a sequence with at least 98% sequence identity or homology.

In one embodiment the vaccine further comprises an mRNA polynucleotide comprising an open reading frame encoding an immunoadjuvant selected from SEQ ID_No 11.

mRNA Modifications

In one embodiment the mRNA polynucleotides described herein comprise one or more modifications relative to the naturally occurring “wild-type” mRNA. Modification refers to modifications of the adenosine (A), guanosine (G), uridine (U), or cytidine (C) nucleotides in at least one of their position, pattern, percent and/or structure. Modifications may comprise naturally-occurring, non-naturally-occurring or a combination of naturally-occurring and non-naturally-occurring modifications. Modifications may be of a sugar, a nucleobase, or an internucleotide linkage (e.g., to a phosphate group). Modifications may be introduced during synthesis or post-synthesis, and may be introduced with chemical synthesis or with a polymerase enzyme. Any of the regions of the mRNA polynucleotide may be modified.

In one embodiment the vaccine comprises an mRNA polynucleotide comprising one or more modifications resulting in reduced degradation of the mRNA after administration relative to an unmodified (i.e. wild-type) polynucleotide.

In one embodiment the vaccine comprises an mRNA polynucleotide comprising one or more modifications resulting in reduced immunogenicity (e.g., a reduced innate response) after administration relative to an unmodified polynucleotide. Modifications of the mRNA polynucleotides described herein may include codon optimisation, capping, for example a five-prime 5′ cap, and uridine modification.

### i) Codon Optimisation

In one embodiment the mRNA polynucleotide may be codon optimized. Codon optimisation ensures the most efficient translation in humans. Codon optimization, in one embodiment, may be used to match codon frequencies in target and host organisms to ensure proper folding; bias G/C content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In one embodiment, the open reading frame (ORF) sequence is optimized using optimization algorithms.

In one embodiment codon-optimized mRNA may, for instance, be one in which the levels of guanosine (G) and/or cytidine (C) residues are enhanced. The G/C-content of nucleic acid molecules may influence the stability of the mRNA. mRNA having an increased amount of guanosine (G) and/or cytidine (C) residues may be functionally more stable than nucleic acids containing a large amount of adenosine (A) and uridine (T) nucleotides. Due to the degeneracy of the genetic code, the modifications work by substituting existing codons for those that promote greater RNA stability without changing the resulting amino acid. The approach is limited to the open reading frame(s) of the mRNA.

In one embodiment codon-optimized mRNA may include uridine depletion relative to an unmodified polynucleotide. Uridine depletion reduces the likelihood of an innate immune response to the mRNA.

### ii) Capping

In one embodiment, the mRNA polynucleotide comprises a five-prime cap (5′ cap). The 5′ cap is a specially altered nucleotide on the 5′ end of the mRNA. mRNA capping may assist with the production of the most biologically active enhancing translation to protein, and least immunogenic mRNA (as in less likely to provoke an innate immune response).

In one embodiment, the mRNA polynucleotide comprises a five-prime cap (5′ cap) selected from CleanCap® available from TriLink Biotechnologies® (e.g. those described in WO 2017/053297). Suitable caps include:
CleanCap® Reagent AG for co-transcriptional capping of mRNA: m7G(5′)ppp(5′)(2′OMeA)pG, or
ARCA (Anti Reverse Cap Analog): 3′-O-Me-m⁷G(5′)ppp(5′)G.

5′ capping prevents degradation of the mRNA in the cytoplasm and promotes ribosome recruitment and protein translation.

In one embodiment the vaccine comprises an mRNA polynucleotide comprising an open reading frame encoding an antigen from an infectious microorganism wherein the mRNA polynucleotide comprises a 5′ cap.

In one embodiment the vaccine comprises an mRNA polynucleotide comprising an open reading frame encoding an antigen from an infectious microorganism or an immunogenic fragment thereof wherein the mRNA polynucleotide comprises a 5′ cap.

In one embodiment the vaccine comprises an mRNA polynucleotide comprising an open reading frame encoding an antigen from an infectious microorganism wherein the mRNA polynucleotide comprises a 5′ cap selected from m7G(5′)ppp(5′)(2′OMeA)pG.

In one embodiment the vaccine comprises an mRNA polynucleotide comprising an open reading frame encoding an antigen from an infectious microorganism or an immunogenic fragment thereof wherein the mRNA polynucleotide comprises a 5′ cap selected from m7G(5′)ppp(5′)(2′OMeA)pG.

In one embodiment the vaccine comprises an mRNA polynucleotide comprising an open reading frame encoding an antigen from an infectious microorganism wherein the mRNA polynucleotide comprises a 5′ cap selected from 3′-O-Me-m7G(5′)ppp(5′) G.

In one embodiment the vaccine comprises an mRNA polynucleotide comprising an open reading frame encoding an antigen from an infectious microorganism or an immunogenic fragment thereof wherein the mRNA polynucleotide comprises a 5′ cap selected from 3′-O-Me-m7G(5′)ppp(5′) G.

iii) Uridine Modification

In one embodiment, the vaccine comprises an mRNA polynucleotide comprising an open reading frame encoding an antigen from an infectious microorganism, wherein the mRNA polynucleotide comprises modified uridine nucleotides. Uridine modification reduces the likelihood of an innate immune response to the mRNA.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides. Uridine modification reduces the likelihood of an innate immune response to the mRNA.

In one embodiment, the vaccine comprises an mRNA polynucleotide comprising an open reading frame encoding an antigen from an infectious microorganism, wherein the mRNA polynucleotide comprises one or more modified uridine nucleotides selected from pseudouridine (ψ), N1-methylpseudouridine (m1ψ), N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises one or more modified uridine nucleotides selected from pseudouridine (ψ), N1-methylpseudouridine (m1ψ), N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine nucleotides.

In one embodiment, the vaccine comprises an mRNA polynucleotide comprising an open reading frame encoding an antigen from an infectious microorganism, wherein the mRNA polynucleotide comprises one or more modified uridine nucleotides selected from N1-methylpseudouridine nucleotide.

In one embodiment, the vaccine comprises an mRNA polynucleotide comprising an open reading frame encoding an antigen from an infectious microorganism or an immunogenic fragment thereof, wherein the mRNA polynucleotide comprises one or more modified uridine nucleotides selected from N1-methylpseudouridine nucleotide.

In one embodiment, the vaccine comprises an mRNA polynucleotide comprising an open reading frame encoding an antigen from an infectious microorganism, wherein the mRNA polynucleotide comprises modified uridine nucleotides wherein at least 80% of the uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 80% of the uridine nucleotides are N1-methylpseudouridine nucleotides.

In one embodiment, the vaccine comprises an mRNA polynucleotide comprising an open reading frame encoding an antigen from an infectious microorganism, wherein the mRNA polynucleotide comprises modified uridine nucleotides wherein at least 90% of the uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 90% of the uridine nucleotides are N1-methylpseudouridine nucleotides.

In one embodiment, the vaccine comprises an mRNA polynucleotide comprising an open reading frame encoding an antigen from an infectious microorganism, wherein the mRNA polynucleotide comprises modified uridine nucleotides wherein at least 95% of the uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 95% of the uridine nucleotides are N1-methylpseudouridine nucleotides.

In one embodiment, the vaccine comprises an mRNA polynucleotide comprising an open reading frame encoding an antigen from an infectious microorganism, wherein the mRNA polynucleotide comprises modified uridine nucleotides wherein at least 97% of the uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 97% of the uridine nucleotides are N1-methylpseudouridine nucleotides.

In one embodiment, the vaccine comprises an mRNA polynucleotide comprising an open reading frame encoding an antigen from an infectious microorganism, wherein the mRNA polynucleotide comprises modified uridine nucleotides wherein at least 99% of the uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 99% of the uridine nucleotides are N1-methylpseudouridine nucleotides.

In one embodiment, the vaccine comprises an mRNA polynucleotide comprising an open reading frame encoding an antigen from an infectious microorganism, wherein the mRNA polynucleotide comprises modified uridine nucleotides wherein all uridine nucleotides are N1-methylpseudouridine nucleotides.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein all uridine nucleotides are N1-methylpseudouridine nucleotides.

### iv) Cytidine Modification

In one embodiment, the vaccine comprises an mRNA polynucleotide comprising modified cytidine nucleotides. Cytidine modification may be employed to impart desirable characteristics such as increased nuclease stability, increased translation or reduced interaction of innate immune receptors with in vitro transcribed RNA.

In one embodiment, the vaccine comprises an mRNA polynucleotide comprising one or more modified cytidine nucleotides selected from N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 5-methylcytidine-5′-triphosphate (5-methyl-CTP), and 2-thio-5-methyl-cytidine.

In one embodiment the mRNA polynucleotide comprises no cytidine modifications.

In one embodiment, the vaccine comprises an mRNA polynucleotide comprising an open reading frame encoding an antigen from an infectious microorganism, wherein the mRNA polynucleotide comprises modified uridine nucleotides wherein at least 80% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 80% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism, wherein the mRNA polynucleotide comprises modified uridine nucleotides wherein at least 90% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 90% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism, wherein the mRNA polynucleotide comprises modified uridine nucleotides wherein at least 95% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 95% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism, wherein the mRNA polynucleotide comprises modified uridine nucleotides wherein at least 97% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 97% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism, wherein the mRNA polynucleotide comprises modified uridine nucleotides wherein at least 99% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein at least 99% of the uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism, wherein the mRNA polynucleotide comprises modified uridine nucleotides wherein all uridine nucleotides are N1-methylpseudouridine nucleotides and the cytidine nucleotides are unmodified.

In one embodiment, the vaccine comprises an mRNA polynucleotide comprising an open reading frame encoding an antigen from an infectious microorganism or an immunogenic fragment thereof, wherein the mRNA polynucleotide comprises modified uridine nucleotides wherein all uridine nucleotides are N1-methylpseudouridine nucleotides and the cytidine nucleotides are unmodified.

In one embodiment the vaccine comprises an mRNA polynucleotide comprising one or more modifications selected from a 5′ cap and uridine modification.

In one embodiment the vaccine comprises an mRNA polynucleotide comprising two modifications selected from a 5′ cap and uridine modification.

In one embodiment the vaccine comprises an mRNA polynucleotide uniformly modified (e.g., fully modified throughout the entire sequence) for a particular modification.

In one embodiment the mRNA polynucleotides described herein comprise one or more modifications selected from a 5′ cap, and uridine modification fully modified throughout the entire sequence.

In one embodiment the vaccine comprises an mRNA polynucleotide comprising one or more modifications selected from a 5′ cap, and uridine modification in one region of the polynucleotide.

### Amphipathic Cell Penetrating Peptide

In accordance with convention, the peptides described herein are drawn “N-terminus” first, i.e. on the left hand side.

In one embodiment the vaccine comprises an amphipathic cell penetrating peptide comprising or consisting of the amino acid sequence WEARLARALARALARHLARALARALRACEA (SEQ ID_No 1) or a sequence with at least 80% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 80% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 80% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 85% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 85% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 85% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 90% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 90% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 90% sequence identity or homology.

In one embodiment the vaccine comprises an amphipathic cell penetrating peptide comprising or consisting of the amino acid sequence WEARLARALARALARHLARALARALRACEA (SEQ ID_No 1) or a sequence with at least 95% sequence identity or homology.

In one embodiment the vaccine comprises an amphipathic cell penetrating peptide comprising the amino acid sequence WEARLARALARALARHLARALARALRACEA (SEQ ID_No 1) or a sequence with at least 95% sequence identity or homology.

In one embodiment the vaccine comprises an amphipathic cell penetrating peptide consisting of the amino acid sequence WEARLARALARALARHLARALARALRACEA (SEQ ID_No 1) or a sequence with at least 95% sequence identity or homology.

In one embodiment the vaccine comprises an amphipathic cell penetrating peptide comprising or consisting of the amino acid sequence WEARLARALARALARHLARALARALRACEA (SEQ ID_No 1).

In one embodiment the vaccine comprises an amphipathic cell penetrating peptide comprising or the amino acid sequence WEARLARALARALARHLARALARALRACEA (SEQ ID_No 1).

In one embodiment the vaccine comprises an amphipathic cell penetrating peptide consisting of the amino acid sequence WEARLARALARALARHLARALARALRACEA (SEQ ID_No 1).

In one embodiment a sequence with at least 80% sequence identity or homology to WEARLARALARALARHLARALARALRACEA (SEQ ID_No 1) consists of less than or equal to 35 amino acid residues.

In one embodiment a sequence with at least 80% sequence identity or homology to WEARLARALARALARHLARALARALRACEA (SEQ ID_No 1) consists of less than or equal to 30 amino acid residues.

In one embodiment a sequence with at least 80% sequence identity or homology to WEARLARALARALARHLARALARALRACEA (SEQ ID_No 1) consists of 26-30 amino acid residues.

In one embodiment a sequence with at least 80% sequence identity or homology to WEARLARALARALARHLARALARALRACEA (SEQ ID_No 1) comprises at least 5 arginine residues (R).

In one embodiment a sequence with at least 80% sequence identity or homology to WEARLARALARALARHLARALARALRACEA (SEQ ID_No 1) comprises at least 6 arginine residues (R).

In one embodiment a sequence with at least 80% sequence identity or homology to WEARLARALARALARHLARALARALRACEA (SEQ ID_No 1) comprises at least 10 alanine residues (A).

In one embodiment a sequence with at least 80% sequence identity or homology to WEARLARALARALARHLARALARALRACEA (SEQ ID_No 1) comprises at least 12 alanine residues (A).

In one embodiment a sequence with at least 80% sequence identity or homology to WEARLARALARALARHLARALARALRACEA (SEQ ID_No 1) comprises at least 5 leucine residues (L).

In one embodiment a sequence with at least 80% sequence identity or homology to WEARLARALARALARHLARALARALRACEA (SEQ ID_No 1) comprises at least 6 leucine residues (L).

In one embodiment a sequence with at least 80% sequence identity or homology to WEARLARALARALARHLARALARALRACEA (SEQ ID_No 1) comprises at least one cysteine residue (C).

In one embodiment a sequence with at least 80% sequence identity or homology to WEARLARALARALARHLARALARALRACEA (SEQ ID_No 1) comprises at least two but no more than three glutamic acid (E) residues.

In one embodiment a sequence with at least 80% sequence identity or homology to WEARLARALARALARHLARALARALRACEA (SEQ ID_No 1) comprises at least 6 arginine residues (R), at least 12 Alanine Residues (A), at least 6 leucine residues (L), optionally at least one cysteine residue (C) and at least two but no more than three glutamic acids residues (E).

In one embodiment a sequence with at least 80% sequence identity or homology to WEARLARALARALARHLARALARALRACEA (SEQ ID_No 1) comprises the consensus sequences EARLARALARALAR and/or LARALARALRA.

In one embodiment a sequence with at least 80% sequence identity or homology to WEARLARALARALARHLARALARALRACEA (SEQ ID_No 1) comprises the consensus sequence EARLARALARALAR.

In one embodiment a sequence with at least 80% sequence identity or homology to WEARLARALARALARHLARALARALRACEA (SEQ ID_No 1) comprises the consensus sequence LARALARALRA.

In one embodiment a sequence with at least 80% sequence identity or homology to WEARLARALARALARHLARALARALRACEA (SEQ ID_No 1) comprises the consensus sequences EARLARALARALAR and LARALARALRA.

In one embodiment a sequence with at least 80% sequence identity or homology to WEARLARALARALARHLARALARALRACEA (SEQ ID_No 1) does not comprise glycine (G).

A Sequence with at Least 80% Sequence Identity or Homology

Any of a variety of sequence alignment methods can be used to determine percent sequence identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Conventional methods include Altschul et al., Bull. Math. Bio. 48:603-16, 1986 and Henikoff and Henikoff, Proc. Natl. Acad. Sci. USA 89:10915-19, 1992 where two amino acid sequences are aligned to optimize the alignment scores using a gap opening penalty of 10, a gap extension penalty of 1, and the “blosum 62” scoring matrix of Henikoff and Henikoff.

“Sequence identity” between two or more sequences is expressed as a percentage and is a function of the number of identical positions shared by the sequences. Thus, sequence identity may be calculated as the number of identical amino acids or nucleotides divided by the total number of amino acids or nucleotides, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Homologous sequences may be characterized as having one or more amino acid or nucleotide substitutions, deletions or additions/insertions. These changes are of a minor nature that do not significantly affect the folding or activity of the peptide. These may be small amino acid or nucleotide substitutions; small deletions; and small terminal extensions or other small additions/insertions. An algorithm (e.g. BLAST) looks for the minimal number of edit operations (inserts, deletes, and substitutions) in order to transform the one sequence into an exact copy of the other sequence and assigns a % similarity (homology).

In one embodiment the term “sequence identity or homology” refers to sequence identity.

In one embodiment the term “sequence identity or homology” refers to sequence homology.

### Nanoparticles

In one embodiment the vaccines described herein comprise or consist of nanoparticles. Nanoparticles may be formed by self-assembly by adding the mRNA polynucleotide, and the amphipathic cell penetrating peptide together in ultrapure water. The formulation may be lyophilised and then reconstituted for administration.

In one embodiment there is provided a nanoparticle formulation comprising:
i) an mRNA polynucleotide comprising an open reading frame encoding an antigen from an infectious microorganism or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment there is provided a vaccine as described herein comprising a nanoparticle formulation of:
i) an mRNA polynucleotide comprising an open reading frame encoding an antigen from an infectious microorganism or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

In one embodiment there is provided a vaccine as described herein formulated in a nanoparticle.

In one embodiment there is provided a nanoparticle comprising a vaccine as described herein.

In one embodiment there is provided a nanoparticle formulation comprising a vaccine as described herein.

In one embodiment there is provided a vaccine as described herein, comprising nanoparticles.

In one embodiment there is provided a vaccine as described herein, comprising nanoparticles with a Z-Average of 30-150 nm. The Z average is the intensity weighted mean hydrodynamic size of the ensemble collection of particles measured by dynamic light scattering (DLS).

In one embodiment there is provided a vaccine as described herein, comprising nanoparticles with a Z-Average of 60-100 nm.

In one embodiment there is provided a vaccine as described herein, comprising nanoparticles with a polydispersity index of =<0.3. The polydispersity index (PI) is a measure of the heterogeneity of a sample based on size.

In one embodiment there is provided a vaccine as described herein, comprising nanoparticles with a polydispersity index of =<0.5.

In one embodiment there is provided a vaccine as described herein, comprising nanoparticles with a net positive charge at a neutral pH.

N:P Ratio of mRNA: Amphipathic Cell Penetrating Peptide

The amount of the RALA peptide to mRNA is indicated by the N:P ratio, and represents the molar ratio of positively charged nitrogen atoms in the peptide to negatively charged phosphates in the mRNA. The mRNA quoted in the ratio may indicate one mRNA, or a mixture of mRNAs depending on the product.

N:P ratio is widely used to describe the contents of peptide or protein based nucleic acid nanoparticles due to its simplicity in comparison to masses. It is the molar ratio of positively charged nitrogen atoms in the amino acids to the negatively charged phosphates contributed by the nucleic acid backbone and can be described as the mass of protein or peptide required to neutralise the charge of 1 μg of nucleic acid. The N:P ratio can be calculated using the following equation:

$$N : P = \frac{MRALA}{MmRNA . CNP}$$

where M_{RALA} is the mass of protein in the nanoparticle, M_{mRNA} is the mass of mRNA in the nanoparticle and C_{NP} is the N:P constant. The N:P constant is the ratio of the positive charge density of the amino acid chain to the negative charge density of the mRNA backbone with charge density being defined as the charge divided by the molecular mass. This N:P constant can be calculated based on the knowledge that arginine is the positively charged amino acids and that the mass and charge of the bases in the mRNA backbone are constant leaving only molecular mass of the protein and charge of the protein as variables. As such the N:P constant calculation can be simplified to:

$$NP 1 = 1 \times \frac{MWRALA}{340 * QRALA} = \frac{3327.98}{340 * 7} = 1.40$$

Where Q_{RALA} is the positive charge provided by the seven arginine residues in the sequence of RALA, and 340 is the average molecular weight of an mRNA nucleoside.

For example an N:P ratio (peptide to mRNA ratio) of 1:1 is 1.40 μg peptide (SEQ ID_No 1):1 μg mRNA. An N:P ratio of 7:1 is 9.79 μg peptide (SEQ ID_No 1):1 μg mRNA, and for an N:P ratio of 9:1 is 12.6 μg peptide (SEQ ID_No 1):1 μg mRNA

In one embodiment, the N:P ratio of the amphipathic cell penetrating peptide:mRNA polynucleotide in the vaccine may be varied. This may have a beneficial effect on the physiochemical characteristics (for example the Z-Average, zeta potential (particle charge), and/or polydispersity index), the cellular uptake, and/or the treatment efficacy.

In one embodiment the N:P ratio of amphipathic cell penetrating peptide:mRNA polynucleotide in the vaccine is about 1-12:1.

In one embodiment the N:P ratio of amphipathic cell penetrating peptide:mRNA polynucleotide is 1-12:1.

In one embodiment the N:P ratio of amphipathic cell penetrating peptide:mRNA polynucleotide in the vaccine is about 5-12:1.

In one embodiment the N:P ratio of amphipathic cell penetrating peptide:mRNA polynucleotide in the vaccine is 5-12:1.

In one embodiment the N:P ratio of amphipathic cell penetrating peptide:mRNA polynucleotide in the vaccine is about 7-10:1.

In one embodiment the N:P ratio of amphipathic cell penetrating peptide:mRNA polynucleotide in the vaccine is 7-10:1.

In one embodiment the N:P ratio of amphipathic cell penetrating peptide:mRNA polynucleotide in the vaccine is about 8.5-9.5:1.

In one embodiment the N:P ratio of amphipathic cell penetrating peptide:mRNA polynucleotide in the vaccine is 8.5-9.5:1.

In one embodiment the N:P ratio of amphipathic cell penetrating peptide:mRNA polynucleotide in the vaccine is 1:1.

In one embodiment the N:P ratio of amphipathic cell penetrating peptide:mRNA polynucleotide in the vaccine is about 1:1.

In one embodiment the N:P ratio of amphipathic cell penetrating peptide:mRNA polynucleotide in the vaccine is 2:1.

In one embodiment the N:P ratio of amphipathic cell penetrating peptide:mRNA polynucleotide in the vaccine is about 2:1.

In one embodiment the N:P ratio of amphipathic cell penetrating peptide:mRNA polynucleotide in the vaccine is 3:1.

In one embodiment the N:P ratio of amphipathic cell penetrating peptide:mRNA polynucleotide in the vaccine is about 3:1.

In one embodiment the N:P ratio of amphipathic cell penetrating peptide:mRNA polynucleotide in the vaccine is 4:1.

In one embodiment the N:P ratio of amphipathic cell penetrating peptide:mRNA polynucleotide in the vaccine is about 4:1.

In one embodiment the N:P ratio of amphipathic cell penetrating peptide:mRNA polynucleotide in the vaccine is 5:1.

In one embodiment the N:P ratio of amphipathic cell penetrating peptide:mRNA polynucleotide in the vaccine is about 5:1.

In one embodiment the N:P ratio of amphipathic cell penetrating peptide:mRNA polynucleotide in the vaccine is 6:1.

In one embodiment the N:P ratio of amphipathic cell penetrating peptide:mRNA polynucleotide in the vaccine is about 6:1.

In one embodiment the N:P ratio of amphipathic cell penetrating peptide:mRNA polynucleotide in the vaccine is 7:1.

In one embodiment the N:P ratio of amphipathic cell penetrating peptide:mRNA polynucleotide in the vaccine is about 7:1.

In one embodiment the N:P ratio of amphipathic cell penetrating peptide:mRNA polynucleotide in the vaccine is 8:1.

In one embodiment the N:P ratio of amphipathic cell penetrating peptide:mRNA polynucleotide in the vaccine is about 8:1.

In one embodiment the N:P ratio of amphipathic cell penetrating peptide:mRNA polynucleotide in the vaccine is 9:1.

In one embodiment the N:P ratio of amphipathic cell penetrating peptide:mRNA polynucleotide in the vaccine is about 9:1.

In one embodiment the N:P ratio of amphipathic cell penetrating peptide:mRNA polynucleotide in the vaccine is 10:1.

In one embodiment the N:P ratio of amphipathic cell penetrating peptide:mRNA polynucleotide in the vaccine is about 10:1.

In one embodiment the N:P ratio of amphipathic cell penetrating peptide:mRNA polynucleotide in the vaccine is 11:1.

In one embodiment the N:P ratio of amphipathic cell penetrating peptide:mRNA polynucleotide in the vaccine is about 11:1.

In one embodiment the N:P ratio of amphipathic cell penetrating peptide:mRNA polynucleotide in the vaccine is 12:1.

In one embodiment the N:P ratio of amphipathic cell penetrating peptide:mRNA polynucleotide in the vaccine is about 12:1.

### Bulking Agents, Cryoprotectants and Agents Inferring Tonicity

In one embodiment, a bulking agent may be added prior to lyophilisation of nanoparticles for transport and storage. Bulking agents are additives that increase the bulk-volume of a product without affecting its properties.

In one embodiment, a cryoprotectant may be added prior to lyophilisation of nanoparticles. A cryoprotectant is a substance used to protect biological material from freezing damage.

In one embodiment, a solute may be added to infer tonicity, e.g. to produce an isotonic formulation once water is added to the formulation. An isotonic formulation possesses the same concentration of solutes as the blood, i.e. 290-310 mOsmol/kg.

In one embodiment, the osmolality of a solution of a vaccine as described herein in water is 10-1000 mOsmol/kg.

In one embodiment, the osmolality of a solution of a vaccine as described herein in water is 100-500 mOsmol/kg.

In one embodiment, the osmolality of a solution of a vaccine as described herein in water is 200-400 mOsmol/kg.

In one embodiment, the osmolality of a solution of a vaccine as described herein in water is 290-310 mOsmol/kg.

In one embodiment, the osmolality of a solution of a vaccine as described herein in water is about 300 mOsmol/kg.

In one embodiment, the osmolality of a solution of a vaccine as described herein in water is 300 mOsmol/kg.

Suitable bulking agents include trehalose, sucrose, mannose, dextrose or any mixture of such agents. These agents may also be employed as cryoprotectants and/or agents to infer tonicity.

In one embodiment, the vaccines described herein additionally comprise trehalose, sucrose, mannose, dextrose or any mixture of such agents.

In one embodiment, the vaccines described herein additionally comprise >85% w/w trehalose, sucrose, mannose, dextrose or any mixture of such agents.

In one embodiment, the vaccines described herein additionally comprise >90% w/w trehalose, sucrose, mannose, dextrose or any mixture of such agents.

In one embodiment, the vaccines described herein additionally comprise >95% w/w trehalose, sucrose, mannose, dextrose or any mixture of such agents.

In one embodiment the vaccine described herein comprises a bulking agent.

In one embodiment the vaccine described herein comprises a bulking agent selected from trehalose, sucrose, mannose and dextrose.

In one embodiment the vaccine described herein comprises trehalose.

In one embodiment the vaccines described herein additionally comprise trehalose.

In one embodiment the vaccines described herein additionally comprise >85% w/w trehalose.

In one embodiment the vaccines described herein additionally comprise >90% w/w trehalose.

In one embodiment the vaccines described herein additionally comprise >95% w/w trehalose.

In one embodiment the vaccines described herein additionally comprise >98% w/w trehalose.

In one embodiment the vaccines described herein additionally comprise about 99% w/w trehalose.

In one embodiment the vaccines described herein additionally comprise 99% w/w trehalose.

In one embodiment the vaccine described herein comprises sucrose.

In one embodiment the vaccines described herein additionally comprise sucrose.

In one embodiment the vaccines described herein additionally comprise >85% w/w sucrose.

In one embodiment the vaccines described herein additionally comprise >90% w/w sucrose.

In one embodiment the vaccines described herein additionally comprise >95% w/w sucrose.

In one embodiment the vaccine described herein comprises mannose.

In one embodiment the vaccines described herein additionally comprise mannose.

In one embodiment the vaccines described herein additionally comprise >85% w/w mannose.

In one embodiment the vaccines described herein additionally comprise >90% w/w mannose.

In one embodiment the vaccines described herein additionally comprise >95% w/w mannose.

In one embodiment the vaccine described herein comprises dextrose.

In one embodiment the vaccine described herein comprises mannose and trehalose.

In one embodiment the vaccine described herein comprises 5-20% w/v of mannose and trehalose. 5-20% w/v of mannose and trehalose means 5-20% w/v (mannose plus trehalose).

In one embodiment the vaccine described herein comprises about 10% w/v of mannose and trehalose.

In one embodiment the vaccine described herein comprises 10% w/v of mannose and trehalose.

In one embodiment the vaccine described herein comprises trehalose and mannose in an 85:15 w/v ratio.

In one embodiment the vaccine described herein comprises trehalose and mannose in about an 85:15 w/v ratio.

In one embodiment the vaccine described herein comprises trehalose and mannose in a 70:30 w/v ratio.

In one embodiment the vaccine described herein comprises trehalose and mannose in about a 70:30 w/v ratio.

In one embodiment the vaccine described herein comprises 5-20% w/v of trehalose and mannose in about an 85:15 w/v ratio.

In one embodiment the vaccine described herein comprises 5-20% w/v of trehalose and mannose in about a 70:30 w/v ratio.

In one embodiment the vaccine described herein comprises about a 10% w/v of trehalose and mannose in about an 85:15 w/v ratio.

In one embodiment the vaccine described herein comprises about a 10% w/v of trehalose and mannose in about a 70:30 w/v ratio.

In one embodiment the vaccine as described herein comprises:
(i) 0.01-1% w/w one or more mRNA polynucleotides comprising open reading frames encoding an antigen from an infectious microorganism or an immunogenic fragment thereof;
(ii) 0.5-5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 89-99% w/w trehalose, sucrose, mannose and/or dextrose.

In one embodiment the vaccine as described herein comprises:
(i) 0.01-1% w/w one or more mRNA polynucleotides comprising open reading frames encoding an HPV antigen or an immunogenic fragment thereof;
(ii) 0.5-2% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 95-99% w/w trehalose.

In one embodiment the vaccine as described herein comprises:
(i) 0.1% w/w one or more mRNA polynucleotides comprising open reading frames encoding an HPV antigen or an immunogenic fragment thereof;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose.

In one embodiment the vaccine as described herein comprises:
(i) about 0.08% w/w one or more mRNA polynucleotides comprising open reading frames encoding an HPV antigen or an immunogenic fragment thereof;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose.

In one embodiment the vaccine as described herein comprises:
(i) 0.08% w/w one or more mRNA polynucleotides comprising open reading frames encoding an HPV antigen or an immunogenic fragment thereof;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose.

In one embodiment the vaccine as described herein comprises:
(i) about 0.08% w/w two or more mRNA polynucleotides comprising open reading frames encoding an HPV antigen or an immunogenic fragment thereof in about equal % w/w;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose.

In one embodiment the vaccine as described herein comprises:
(i) 0.08% w/w two or more mRNA polynucleotides comprising open reading frames encoding an HPV antigen or an immunogenic fragment thereof in equal % w/w;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose.

In one embodiment the vaccine as described herein comprises:
(i) about 0.08% w/w three mRNA polynucleotides comprising open reading frames encoding an HPV antigen or an immunogenic fragment thereof in about equal % w/w;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose.

In one embodiment the vaccine as described herein comprises:
(i) 0.08% w/w three mRNA polynucleotides comprising open reading frames encoding an HPV antigen or an immunogenic fragment thereof in equal % w/w;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose.

### Formulations in Water

The vaccines described herein may be conveniently formulated in water, particularly ultrapure water, for ease of administration, particularly via intravenous injection.

In one embodiment the vaccine as described herein comprises:
(i) 0.01-1% w/w one or more mRNA polynucleotides comprising open reading frames encoding an antigen from an infectious microorganism or an immunogenic fragment thereof;
(ii) 0.5-5% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 89-99% w/w trehalose, sucrose, mannose and/or dextrose;
   formulated as an isotonic formulation in water with an osmolality of 290-310 mOsmol/kg.

In one embodiment the vaccine as described herein comprises:
(i) 0.01-1% w/w one or more mRNA polynucleotides comprising open reading frames encoding an HPV antigen or an immunogenic fragment thereof;
(ii) 0.5-2% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 95-99% w/w trehalose;
   formulated as an isotonic formulation in water with an osmolality of 290-310 mOsmol/kg.

In one embodiment the vaccine as described herein comprises:
(i) 0.1% w/w one or more mRNA polynucleotides comprising open reading frames encoding an HPV antigen or an immunogenic fragment thereof;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose;
   formulated as an isotonic formulation in water with an osmolality of 290-310 mOsmol/kg.

In one embodiment the vaccine as described herein comprises:
(i) about 0.08% w/w one or more mRNA polynucleotides comprising open reading frames encoding an HPV antigen or an immunogenic fragment thereof;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose;
   formulated as an isotonic formulation in water with an osmolality of 290-310 mOsmol/kg.

In one embodiment the vaccine as described herein comprises:
(i) 0.08% w/w one or more mRNA polynucleotides comprising open reading frames encoding an HPV antigen or an immunogenic fragment thereof;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose;
   formulated as an isotonic formulation in water with an osmolality of 290-310 mOsmol/kg.

In one embodiment the vaccine as described herein comprises:
(i) about 0.08% w/w two or more mRNA polynucleotides comprising open reading frames encoding an HPV antigen or an immunogenic fragment thereof in about equal % w/w;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose;
   formulated as an isotonic formulation in water with an osmolality of 290-310 mOsmol/kg.

In one embodiment the vaccine as described herein comprises:
(i) 0.08% w/w two or more mRNA polynucleotides comprising open reading frames encoding an HPV antigen or an immunogenic fragment thereof in equal % w/w;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose;
   formulated as an isotonic formulation in water with an osmolality of 290-310 mOsmol/kg.

In one embodiment the vaccine as described herein comprises:
(i) about 0.08% w/w three mRNA polynucleotides comprising open reading frames encoding an HPV antigen or an immunogenic fragment thereof in about equal % w/w;
(ii) about 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) about 99% w/w trehalose;
   formulated as an isotonic formulation in water with an osmolality of 290-310 mOsmol/kg.

In one embodiment the vaccine as described herein comprises:
(i) 0.08% w/w three mRNA polynucleotides comprising open reading frames encoding an HPV antigen or an immunogenic fragment thereof in equal % w/w;
(ii) 1% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 99% w/w trehalose;
   formulated as an isotonic formulation in water with an osmolality of 290-310 mOsmol/kg.

### Automated Formulation

In one embodiment, the vaccines are prepared via an automated controllable mixing system, for example an automated microfluidics system, for example Precision Nanosystems Ignite NanoAssemblr. This technology has the potential to control both the mixing rate and the mixing ratios during formulation of the nanoparticles, resulting in a reduction in Z-average particle size, resulting in an additional decrease in the polydispersity index when compared to manual formulation methods.

Microfluidics refers to the behaviour, precise control, and manipulation of fluids that are geometrically constrained to a small scale (typically sub-millimetre) at which surface forces dominate volumetric forces.

In one embodiment the vaccines described herein are prepared via an automated controlled mixing system.

In one embodiment there is provided a method of preparing a nanoparticle formulation which comprises formulating a solution of an mRNA polynucleotide comprising an open reading frame encoding an antigen from an infectious microorganism or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system, particularly an automated microfluidics system.

In one embodiment there is provided a method of preparing a nanoparticle formulation which comprises formulating a solution of an mRNA polynucleotide comprising an open reading frame encoding an antigen from an infectious microorganism or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is 1:3.

In one embodiment there is provided a method of preparing a nanoparticle formulation which comprises formulating a solution of an mRNA polynucleotide comprising an open reading frame encoding an antigen from an infectious microorganism or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is about 1:3.

In one embodiment there is provided a method of preparing a nanoparticle formulation which comprises formulating a solution of an mRNA polynucleotide comprising an open reading frame encoding an antigen from an infectious microorganism or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is 1:5 to 5:1.

In one embodiment there is provided a method of preparing a nanoparticle formulation which comprises formulating a solution of an mRNA polynucleotide comprising an open reading frame encoding an antigen from an infectious microorganism or an immunogenic fragment thereof; with an amphipathic cell penetrating peptide comprising or consisting of the amino acid sequence WEARLARALARALARHLARALARALRACEA (SEQ ID_No 1) or a sequence with at least 80% sequence identity or homology in an automated controlled mixing system wherein the flow rate ratio of mRNA:peptide is about 1:5 to 5:1.

### Formulation

Nanoparticles may be formed by self-assembly by adding the mRNA and the amphipathic cell penetrating peptide together in ultrapure water with instantaneous formulation occurring. The resulting vaccine may be lyophilised for transport and storage, and then rehydrate in water for use.

The vaccine described herein may be employed in various routes of administration, for example oral, nasal, inhaled, rectal, topically, percutaneous, intravitreal, intravenous, intramuscular, or intradermal administration, particularly intradermal or intramuscular administration. Intradermal and intramuscular routes of administration may maximise delivery to dendritic cells which are highly prevalent near the skin surface.

In one embodiment, the vaccine of the present disclosure may be employed in an injectable formulation, for example an intradermal injection.

In one embodiment, the vaccine of the present disclosure may be employed in an injectable formulation, for example an intramuscular injection.

In one embodiment, the vaccine of the present disclosure may be employed in an injectable formulation, for example an intravenous injection.

In one embodiment, the vaccine of the present disclosure may be employed in an injectable formulation, for example an intradermal patch. The surface of an intradermal patch is covered in tiny microneedles which dissolve in the body. The patch can be applied painlessly, like a plaster, and allows the vaccine to quickly overcome the outer skin barrier and be delivered straight into the area with most dendritic cells.

### Dose

The exact amount required for effective vaccination will vary from subject to subject, depending on the species, age, and general condition of the subject, the severity of the disease, the particular composition, its mode of administration, its mode of activity, the desired prophylactic and/or therapeutic effect and the like. Vaccines compositions are typically formulated in dosage unit form for ease of administration and uniformity of dosage. It will be understood, however, that the total dose of vaccine compositions may be decided by the attending physician within the scope of sound medical judgment. Vaccines may be administered more than once, for example two or three times on separate occasions, in order to maximise the effect.

In one embodiment the vaccine may be administered at a dosage of between 0.1 μg/kg and 500 μg/kg to the subject.

In one embodiment the vaccine may be administered at a dosage of between 1 μg/kg and 500 μg/kg to the subject.

In one embodiment, the vaccine described herein comprises 10-150 μg of mRNA polynucleotide.

In one embodiment, the vaccine described herein comprises about 100 μg mRNA polynucleotide.

In one embodiment, the vaccine described herein comprises 100 μg mRNA polynucleotide.

In one embodiment, the vaccine is administered twice.

In one embodiment, the vaccine is administered three times.

In one embodiment, the vaccine described herein comprises 10-150 μg mRNA polynucleotide and is administered three times.

In one embodiment, the vaccine described herein comprises about 100 μg mRNA polynucleotide and is administered three times.

In one embodiment, the vaccine described herein comprises 100 μg mRNA polynucleotide and is administered three times.

In one embodiment, the vaccine described herein comprises at least two mRNA polynucleotides comprising open reading frames and encoding an antigen from an infectious microorganism or an immunogenic fragment thereof, wherein the dosage for the vaccine is a combined therapeutic dosage, and wherein the dosage of each individual mRNA polynucleotide is a sub therapeutic dosage.

### Age

In one embodiment, the vaccine described herein may be administered to a subject under 5 years old.

In one embodiment, the vaccine described herein may be administered to a subject aged 5-11 years old.

In one embodiment, the vaccine described herein may be administered to a subject aged 9-45 years old.

In one embodiment, the vaccine described herein may be administered to a subject aged 12-15 years old.

In one embodiment, the vaccine described herein may be administered to a subject aged 16 or 17 years old.

In one embodiment, the vaccine described herein may be administered to a subject aged 17 years or younger.

In one embodiment, the vaccine described herein may be administered to a subject aged 18 years or older.

In one embodiment, the vaccine described herein may be administered to a subject aged 60 years or older.

### Vaccine Effectiveness

Vaccine efficacy may be assessed using standard analyses (see, e.g., Weinberg et al., J Infect Dis. 2010 Jun. 1: 201 (11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using Standard analyses (see. e.g., Weinberg et al., J Infect Dis. 2010 Jun. 1; 201 (11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In one embodiment, the vaccine immunizes the subject for up to 2 years.

In one embodiment, the vaccine immunizes the subject for more than 2 years, more than 3 years, more than 4 years, or for 5-10 years.

An “effective amount” of a vaccine may be determined by the target tissue, target cell type, means of administration, physical characteristics of the components of the vaccine, and other determinants. In general, an effective amount of the vaccine composition provides an induced or boosted immune response as a function of antigen production in the cell. Increased antigen production may be demonstrated by increased cell transfection, increased protein translation, decreased nucleic acid degradation, or altered antigen specific immune response of the host cell. In one embodiment an “effective amount” is a therapeutically effective amount.

### Charge Density

The mean surface charge density of nanoparticles may be a contributing factor to their toxicity by promoting oxidative stress mechanisms which in turn can promote mitochondrial dysfunction and viability loss.

The charge density may be measured by polyelectrolytic titration using methods described in Ritz et al, Biomacromolecules. 2015 Apr. 13; 16 (4):1311-21. doi: 10.1021/acs.biomac.5b00108. Epub 2015 Apr. 3 and Weiss et al, J Nanobiotechnology. 2021 Jan. 6; 19 (1):5. doi: 10.1186/s12951-020-00747-7.

Polyelectrolytic titration may be performed using poly(acrylic acid) (PAA) 0.01 M at pH 7.4 and addition of PAA to nanoparticles and measuring the charge creates a sigmoidal curve of which the volume (V) can be derived from the equivalence point. In conjunction, the mass of peptide in the system (w) and concentration of PAA (0.01 M) used, is used to calculate the average charge density of the nanoparticle (Qₑₖ).

In one embodiment, the mean surface charge density of the nanoparticles comprising vaccines as described herein is ≤2 μmol/mg at 20° C. Mean surface charge density figures were measured using an Orion STAR® multi parameter bench meter.

In one embodiment, the mean surface charge density of the nanoparticles comprising vaccines as described herein is ≤1.5 μmol/mg at 20° C.

In one embodiment, the mean surface charge density of the nanoparticles comprising vaccines as described herein is about 1 μmol/mg at 20° C.

In one embodiment, the mean surface charge density of the nanoparticles comprising vaccines as described herein is 1 μmol/mg at 20° C.

### Uses

In one embodiment there is provided a vaccine as described herein for use as a medicament.

In one embodiment there is provided the use of a vaccine as described herein as a medicament.

In one embodiment there is provided a vaccine as described herein for use in therapy.

As used herein, the terms “treatment” and “treat” refer to preventing, reversing, alleviating, delaying the onset of, or inhibiting the progress of a disease or disorder, or one or more symptoms thereof, as described herein. In one embodiment, treatment may be conducted before infection has occurred. In one embodiment, treatment may be conducted in subjects exposed to infection. In one embodiment, treatment may be conducted in subjects at risk of infection. In one embodiment, treatment may be conducted after one or more symptoms have developed. In other embodiments, treatment may be conducted in the absence of symptoms. For example, treatment may be conducted to a susceptible individual prior to the onset of symptoms (e.g. in light of a history of symptoms and/or in light of genetic or other susceptibility factors and/or indicative diagnostic tests). Treatment may also be continued after symptoms have resolved, for example to present or delay their recurrence.

Herein where use in the treatment of cancer is described, this may be cancer in early stage, actively progressing, metastatic and/or drug-resistant cancer. In one embodiment where cancer is referred to, the cancer is early cancer. In one embodiment where cancer is referred to, the cancer is locally advanced cancer. In one embodiment where cancer is referred to, the cancer is locally advanced and/or metastatic cancer. In one embodiment where cancer is referred to, the cancer is metastatic cancer. In one embodiment where cancer is referred to the cancer is invasive cancer.

In one embodiment there is provided a vaccine as described herein for use as a therapeutic vaccine.

In one embodiment there is provided a vaccine as described herein for use as a prophylactic vaccine.

In one embodiment there is provided a vaccine as described herein for use as a therapeutic and prophylactic vaccine.

In one embodiment there is provided a vaccine as described herein for use in creating, maintaining or restoring antigenic memory to a virus strain.

In one embodiment there is provided a vaccine as described herein for use in inducing an antigen specific immune response in a subject, particularly a CD8+ T cell response.

In one embodiment there is provided a vaccine as described herein for use in delivering mRNA comprising an open reading frame encoding an antigen from an infectious microorganism or an immunogenic fragment thereof, into a cell.

In one embodiment there is provided a vaccine as described herein for use in delivering mRNA comprising an open reading frame encoding an antigen from an infectious microorganism or an immunogenic fragment thereof, to a target tissue of the immune system, particularly the lymph nodes, spleen and/or bone marrow.

In one embodiment there is provided a vaccine as described herein for use in producing an antigen from an infectious microorganism or an immunogenic fragment thereof, in a cell, tissue or organism.

In one embodiment there is provided a vaccine as described herein for use in the treatment of cancer.

In one embodiment there is provided a vaccine as described herein for use in the treatment of HPV-related cancer.

In one embodiment there is provided a vaccine as described herein for use in the treatment of squamous cell carcinoma and adenocarcinoma.

In one embodiment there is provided a vaccine as described herein for use in the treatment of cervical cancer, oropharyngeal and other head and neck cancer, anal cancer, penile cancer, vaginal cancer and/or vulvar cancer.

In one embodiment there is provided a vaccine as described herein for use in the treatment of precancerous or dysplastic lesions.

In one embodiment there is provided a vaccine as described herein for use in the treatment of cervical, vulvar, vaginal, and/or anal intraepithelial neoplasia.

In one embodiment there is provided a vaccine as described herein for use in the treatment of sexually transmitted diseases.

In one embodiment there is provided a vaccine as described herein for use in the treatment of genital warts.

In one embodiment there is provided a vaccine as described herein for use in the treatment of HPV infection.

In one embodiment there is provided a vaccine as described herein for use in the treatment of mucosal HPV infection.

In one embodiment there is provided a vaccine as described herein for use in the treatment of cutaneous HPV infection.

In one embodiment there is provided a vaccine as described herein for use in the treatment of HPV types 6, 11, 8, 16 and/or 18 infection.

### Pharmaceutical Compositions

In one embodiment there is provided a pharmaceutical composition which comprises a vaccine as described herein for use in an intradermal injection.

In one embodiment there is provided a pharmaceutical composition which comprises a vaccine as described herein for use in an intramuscular injection.

In one embodiment there is provided a pharmaceutical composition which comprises a vaccine as described herein for use as a therapeutic vaccine.

In one embodiment there is provided a pharmaceutical composition which comprises a vaccine as described herein for use as a prophylactic vaccine.

In one embodiment there is provided a pharmaceutical composition which comprises a vaccine as described herein for use as a therapeutic and prophylactic vaccine.

In one embodiment there is provided a pharmaceutical composition which comprises a vaccine as described herein for use in creating, maintaining or restoring antigenic memory to a virus strain.

In one embodiment there is provided a pharmaceutical composition which comprises a vaccine as described herein for use in inducing an antigen specific immune response in a subject, particularly a CD8+ T cell response.

In one embodiment there is provided a pharmaceutical composition which comprises a vaccine as described herein for use in delivering mRNA comprising an open reading frame encoding an antigen from an infectious microorganism or an immunogenic fragment thereof, into a cell.

In one embodiment there is provided a pharmaceutical composition which comprises a vaccine as described herein for use in delivering mRNA comprising an open reading frame encoding an antigen from an infectious microorganism or an immunogenic fragment thereof, to a target tissue of the immune system, particularly the lymph nodes, spleen and/or bone marrow.

In one embodiment there is provided a pharmaceutical composition which comprises a vaccine as described herein for use in producing an antigen from an infectious microorganism or an immunogenic fragment thereof, in a cell, tissue or organism.

In one embodiment there is provided a pharmaceutical composition which comprises a vaccine as described herein for use in the treatment of cancer.

In one embodiment there is provided a pharmaceutical composition which comprises a vaccine as described herein for use in the treatment of HPV-related cancer.

In one embodiment there is provided a pharmaceutical composition which comprises a vaccine as described herein for use in the treatment of squamous cell carcinoma and adenocarcinoma.

In one embodiment there is provided a pharmaceutical composition which comprises a vaccine as described herein for use in the treatment of cervical cancer, oropharyngeal and other head and neck cancer, anal cancer, penile cancer, vaginal cancer and/or vulvar cancer.

In one embodiment there is provided a pharmaceutical composition which comprises a vaccine as described herein for use in the treatment of precancerous or dysplastic lesions.

In one embodiment there is provided a pharmaceutical composition which comprises a vaccine as described herein for use in the treatment of cervical, vulvar, vaginal, and/or anal intraepithelial neoplasia.

In one embodiment there is provided a pharmaceutical composition which comprises a vaccine as described herein for use in the treatment of sexually transmitted diseases.

In one embodiment there is provided a pharmaceutical composition which comprises a vaccine as described herein for use in the treatment of genital warts.

In one embodiment there is provided a pharmaceutical composition which comprises a vaccine as described herein for use in the treatment of HPV infection.

In one embodiment there is provided a pharmaceutical composition which comprises a vaccine as described herein for use in the treatment of mucosal HPV infection.

In one embodiment there is provided a pharmaceutical composition which comprises a vaccine as described herein for use in the treatment of cutaneous HPV infection.

In one embodiment there is provided a pharmaceutical composition which comprises a vaccine as described herein for use in the treatment of HPV types 6, 11, 8, 16 and/or 18 infection.

### Methods of Treatment

In one embodiment there is provided a method of intradermal injection which comprises administering a vaccine as described herein.

In one embodiment there is provided a method of intravenous injection which comprises administering a vaccine as described herein.

In one embodiment there is provided a method of therapeutic vaccination in a warm-blooded animal, such as man, which comprises administering to said animal an effective amount of a vaccine as described herein.

In one embodiment there is provided a method of prophylactic vaccination in a warm-blooded animal, such as man, which comprises administering to said animal an effective amount of a vaccine as described herein.

In one embodiment there is provided a method of therapeutic and prophylactic vaccination in a warm-blooded animal, such as man, which comprises administering to said animal an effective amount of a vaccine as described herein.

In one embodiment there is provided a method of creating, maintaining or restoring antigenic memory to a virus strain in a warm-blooded animal, such as man, which comprises administering to said animal an effective amount of a vaccine as described herein.

In one embodiment there is provided a method of inducing an antigen specific immune response in a subject, particularly a CD8+ T cell response in a warm-blooded animal, such as man, which comprises administering to said animal an effective amount of a vaccine as described herein.

In one embodiment there is provided a method of delivering mRNA comprising an open reading frame encoding an antigen from an infectious microorganism or an immunogenic fragment thereof, into a cell in a warm-blooded animal, such as man, which comprises administering to said animal an effective amount of a vaccine as described herein.

In one embodiment there is provided a method of delivering mRNA comprising an open reading frame encoding an antigen from an infectious microorganism or an immunogenic fragment thereof, to a target tissue of the immune system, particularly the lymph nodes, spleen and/or bone marrow in a warm-blooded animal, such as man, which comprises administering to said animal an effective amount of a vaccine as described herein.

In one embodiment there is provided a method of producing an antigen from an infectious microorganism or an immunogenic fragment thereof, in a cell, tissue or organism in a warm-blooded animal, such as man, which comprises administering to said animal an effective amount of a vaccine as described herein.

In one embodiment there is provided a method of treating cancer in a warm-blooded animal, such as man, which comprises administering to said animal an effective amount of a vaccine as described herein.

In one embodiment there is provided a method of treating HPV-related cancer in a warm-blooded animal, such as man, which comprises administering to said animal an effective amount of a vaccine as described herein.

In one embodiment there is provided a method of treating squamous cell carcinoma and adenocarcinoma in a warm-blooded animal, such as man, which comprises administering to said animal an effective amount of a vaccine as described herein.

In one embodiment there is provided a method of treating cervical cancer, oropharyngeal and other head and neck cancer, anal cancer, penile cancer, vaginal cancer and/or vulvar cancer in a warm-blooded animal, such as man, which comprises administering to said animal an effective amount of a vaccine as described herein.

In one embodiment there is provided a method of treating precancerous or dysplastic lesions in a warm-blooded animal, such as man, which comprises administering to said animal an effective amount of a vaccine as described herein.

In one embodiment there is provided a method of treating cervical, vulvar, vaginal, and/or anal intraepithelial neoplasia in a warm-blooded animal, such as man, which comprises administering to said animal an effective amount of a vaccine as described herein.

In one embodiment there is provided a method of treating sexually transmitted diseases in a warm-blooded animal, such as man, which comprises administering to said animal an effective amount of a vaccine as described herein.

In one embodiment there is provided a method of treating genital warts in a warm-blooded animal, such as man, which comprises administering to said animal an effective amount of a vaccine as described herein.

In one embodiment there is provided a method of treating HPV infection in a warm-blooded animal, such as man, which comprises administering to said animal an effective amount of a vaccine as described herein.

In one embodiment there is provided a method of treating mucosal HPV infection in a warm-blooded animal, such as man, which comprises administering to said animal an effective amount of a vaccine as described herein.

In one embodiment there is provided a method of treating cutaneous HPV infection in a warm-blooded animal, such as man, which comprises administering to said animal an effective amount of a vaccine as described herein.

In one embodiment there is provided a method of treating HPV types 6, 11, 8, 16 and/or 18 infection in a warm-blooded animal, such as man, which comprises administering to said animal an effective amount of a vaccine as described herein.

### Use of a Vaccine

In one embodiment there is provided the use of a vaccine as described herein for the manufacture of a medicament for intradermal injection.

In one embodiment there is provided the use of a vaccine as described herein for the manufacture of a medicament for intramuscular injection.

In one embodiment there is provided the use of a vaccine as described herein for the manufacture of a medicament for therapeutic vaccination.

In one embodiment there is provided the use of a vaccine as described herein for the manufacture of a medicament for prophylactic vaccination.

In one embodiment there is provided the use of a vaccine as described herein for the manufacture of a medicament for therapeutic and prophylactic vaccination.

In one embodiment there is provided the use of a vaccine as described herein for the manufacture of a medicament for creating, maintaining or restoring antigenic memory to a virus strain.

In one embodiment there is provided the use of a vaccine as described herein for the manufacture of a medicament for inducing an antigen specific immune response in a subject, particularly a CD8+ T cell response.

In one embodiment there is provided the use of a vaccine as described herein for the manufacture of a medicament for delivering mRNA comprising an open reading frame encoding an antigen from an infectious microorganism or an immunogenic fragment thereof, into a cell.

In one embodiment there is provided the use of a vaccine as described herein for the manufacture of a medicament for delivering mRNA comprising an open reading frame encoding an antigen from an infectious microorganism or an immunogenic fragment thereof, to a target tissue of the immune system, particularly the lymph nodes, spleen and/or bone marrow.

In one embodiment there is provided the use of a vaccine as described herein for the manufacture of a medicament for producing an antigen from an infectious microorganism or an immunogenic fragment thereof, in a cell, tissue or organism.

In one embodiment there is provided the use of a vaccine as described herein for the manufacture of a medicament for the treatment of cancer.

In one embodiment there is provided the use of a vaccine as described herein for the manufacture of a medicament for the treatment of HPV-related cancer.

In one embodiment there is provided the use of a vaccine as described herein for the manufacture of a medicament for the treatment of squamous cell carcinoma and adenocarcinoma.

In one embodiment there is provided the use of a vaccine as described herein for the manufacture of a medicament for the treatment of cervical cancer, oropharyngeal and other head and neck cancer, anal cancer, penile cancer, vaginal cancer and/or vulvar cancer.

In one embodiment there is provided the use of a vaccine as described herein for the manufacture of a medicament for the treatment of precancerous or dysplastic lesions.

In one embodiment there is provided the use of a vaccine as described herein for the manufacture of a medicament for the treatment of cervical, vulvar, vaginal, and/or anal intraepithelial neoplasia.

In one embodiment there is provided the use of a vaccine as described herein for the manufacture of a medicament for the treatment of sexually transmitted diseases.

In one embodiment there is provided the use of a vaccine as described herein for the manufacture of a medicament for the treatment of genital warts.

In one embodiment there is provided the use of a vaccine as described herein for the manufacture of a medicament for the treatment of HPV infection.

In one embodiment there is provided the use of a vaccine as described herein for the manufacture of a medicament for the treatment of mucosal HPV infection.

In one embodiment there is provided the use of a vaccine as described herein for the manufacture of a medicament for the treatment of cutaneous HPV infection.

In one embodiment there is provided the use of a vaccine as described herein for the manufacture of a medicament for the treatment of HPV types 6, 11, 8, 16 and/or 18 infection.

### Kits

In one embodiment there is provided a kit comprising:
a) a vaccine as described herein;
b) container means for containing said vaccine.

In one embodiment there is provided a kit comprising:
a) a vaccine as described herein;
b) container means for containing said vaccine; and optionally
c) instructions for use.

### DESCRIPTION OF THE FIGURES

FIG. 1 Shows particle size (Z-Average) plots and Zeta potential for RALA/E7 mRNA nanoparticles (Compounds 001, 002, 003, 004 and 005) produced by manual formulation, and a representative size distribution spectrum of nanoparticles comprising 10 μg E6 and 10 μg E7 at N:P7 (Compound 021). Results from Example 4.

FIG. 2 represents the accumulation of fluorescence signal in the skin draining lymph nodes of C57BL/6 mice vaccinated intradermally via the ear. The fluorescent signal from Cy5-EGFP mRNA complexed in N:P 7 (Compound 010) nanoparticles migrated from the ear more quickly, and was evident in the draining lymph nodes to a higher degree than that from the other N:P ratios tested (Compounds 006, 007, 008 and 009). Results from Example 5.

FIG. 3 shows Cy5 content from immune cells from disaggregated lymph nodes (CD11c+ dendritic cells and F4/80+ macrophages). Results from Example 5.

FIG. 4 Shows the dendritic cells that take up Compound 010 nanoparticles are of a migratory phenotype. Results from Example 5.

FIG. 5 Shows E7 protein production by Compounds 011, 012, 013, 014 and 015 following transfection into HEK-293 cells (analysed by in-cell ELISA). Results from Example 6.

FIG. 6 Shows the results of an in vivo study to evaluate the IFNγ response of each of Compounds 011, 012, 013, 014 and 015. Results from Example 6.

FIG. 7 Shows the number of IFNγ and IL-2 spot-forming units (SFU) after 48 h from an experiment where C57BL/6 mice were vaccinated with Compound 013, 016 or 017 that had been lyophilized using trehalose alone, mannose alone, or a trehalose-mannose blend (85%/15%) respectively in a two-dose regimen. Results from Example 7.

FIG. 8 Shows intracellular IFNγ, IL-2 and TNFα content by flow cytometry. Results from Example 7.

FIG. 9 Shows IFNγ (Th1) and interleukin 4 (IL-4; Th2) content. Results from Example 7.

FIG. 10 Shows the number of IFNγ and IL-2 spot-forming units (SFU). Results from Example 8.

FIG. 11 Shows IFNγ content. Results from Example 8.

FIG. 12 Shows tumour volume. Results from Example 9.

FIG. 13 Shows survival proportions of mice treated with Compound 019, 020, 021, 022, 023 or 024. Results from Example 9.

FIG. 14 Shows tumour volume. Results from Example 9.

FIG. 15 Shows survival proportions of mice treated with Compound 019, 020, 021, 022, 023 or 024. Results from Example 9.

FIG. 16 Shows the number of IFNγ spot-forming units was counted. Results from Example 10.

FIG. 17 Shows CD62L⁺ and E7 Dex+ cells. Results from Example 11.

FIG. 18 Shows tumour volume of mice treated with Compound 031. Results from Example 12.

FIG. 19 Shows survival proportions of mice treated with Compound 031. Results from Example 12.

FIG. 20 Shows immunogenicity of Compound 031, 033, 034, 035, 036 and 037 in vaccinated mice, as determined using IFNγ ELISpot. Results from Example 13.

FIG. 21 Shows immunogenicity of Compound 035 in vaccinated mice, as determined using IFNγ ELISpot. Results from Example 14.

FIG. 22 Shows tumour volume of mice treated with Compound 035. Results from Example 15.

FIG. 23 Shows survival proportions of mice treated with Compound 035. Results from Example 15.

FIG. 24 Shows tumour volume of mice treated with Compound 035. Results from Example 16.

FIG. 25 Shows survival proportions of mice treated with Compound 035. Results from Example 16.

FIG. 26 Shows tumour volume of mice treated with Compound 035 or 039. Results from Example 17.

FIG. 27 Shows survival proportions of mice treated with Compound 035 or 039. Results from Example 17.

FIG. 28 Shows immunogenicity of Compound 035 or 040 in vaccinated mice, as determined using IFNγ ELISpot. Results from Example 18.

FIG. 29 Shows immunogenicity of Compound 035 in vaccinated mice, as determined using IFNγ ELISpot. Results from Example 19.

FIG. 30 Shows uptake of Compound 041 following intravenous delivery in mice.

Asterix in the figures represent statistical significance.

### EXAMPLES

### General Experimental

In the Example section, the following applies:
RALA refers to the peptide WEARLARALARALARHLARALARALRACEA (SEQ ID_No 1) and was obtained from Biomatik®, Canada, or Bachem, Switzerland in lyophilised powder form;
mRNA (E6) refers to a mRNA with 5′ and 3′ untranslated regions, a poly-A tail comprising 80 or 120 adenosines and a basic open reading frame sequence of SEQ ID_No 2 (uridine and cytidine modifications may differ and are noted in the table below), with a 5′ CleanCap® purchased from Trilink Biotechnologies®;
mRNA (E7) refers to a mRNA with 5′ and 3′ untranslated regions, a poly-A tail comprising 80 or 120 adenosines and a basic open reading frame sequence of SEQ ID_No 3 (uridine and cytidine modifications may differ and are noted in the table below), with a 5′ CleanCap® or ARCA cap purchased from Trilink Biotechnologies®; mRNA (E1) refers to a mRNA with 5′ and 3′ untranslated regions, a poly-A tail comprising 80 or 120 adenosines and a basic open reading frame sequence of SEQ ID_No 4 (uridine and cytidine modifications may differ and are noted in the table below), with a 5′ CleanCap® purchased from Trilink Biotechnologies®;
mRNA (IL-15) refers to a mRNA with 5′ and 3′ untranslated regions, a poly-A tail comprising 120 adenosines and a basic open reading frame sequence of SEQ ID_No 8 (uridine and cytidine modifications may differ and are noted in the table below), with a 5′ CleanCap® purchased from Trilink Biotechnologies®;
All mRNAs were purchased from Trilink Biotechnologies and were used without further Modification®;
TC-1 cells were obtained from Johns Hopkins University Cell Repository, USA;
C3.43 cells were obtained from The University of Southern California;
C57BL/6 mice were obtained from Envigo®;
HEK-293 cells were obtained from ATCC;
Cy5-EGFP refers to cyanine 5 fluorescently tagged mRNA that encodes enhanced green fluorescent protein obtained from Trilink Biotechnologies®;
EGFP mRNA refers to mRNA that encodes enhanced green fluorescent protein obtained from Trilink Biotechnologies®; and
NCTC-929 mouse fibroblasts were obtained from ATCC.

### Example 1: Preparation of RALA Peptide

RALA was reconstituted with molecular grade water to a desired concentration, aliquoted and stored at −20° C. until further use. An aliquot was taken as needed and defrosted on ice. Aliquots were not re-frozen once they had been defrosted.

### Example 2: Nanoparticle Formulation

### 2.1: Manual Formulation

RALA/mRNA nanoparticles were formulated at various N:P ratios by first adding necessary volumes of ultrapure water to mRNA in solution to achieve a concentration of 1 μg/μl. The corresponding volumes of peptide solution at a concentration of 10 μg/μl (Table 6) were added to the diluted mRNA solution. The mixture was pipetted up and down approximately 5-10 times to ensure homogenous mixing. Nanoparticles formed spontaneously in solution.

TABLE 6
Manual preparation of RALA/mRNA nanoparticles
at different N:P ratios.

| | | | Volumes (μl) | | |
|---|---|---|---|---|---|
| N:P | Masses (μg) | | mRNA at 1 | RALA at | |
| ratio | mRNA | RALA | μg/μl | 10 μg/μl | Water |
| 0:1 | 20 | 0.00 | 20 | 0.00 | 180.00 |
| 1:1 | 20 | 27.97 | 20 | 2.80 | 177.20 |
| 2:1 | 20 | 55.93 | 20 | 5.59 | 174.41 |
| 3:1 | 20 | 83.90 | 20 | 8.39 | 171.61 |
| 4:1 | 20 | 111.86 | 20 | 11.19 | 168.81 |
| 5:1 | 20 | 139.83 | 20 | 13.98 | 166.02 |
| 6:1 | 20 | 167.80 | 20 | 16.78 | 163.22 |
| 7:1 | 20 | 195.76 | 20 | 19.58 | 160.42 |
| 8:1 | 20 | 223.73 | 20 | 22.37 | 157.63 |
| 9:1 | 20 | 251.70 | 20 | 25.17 | 154.83 |
| 10:1 | 20 | 279.66 | 20 | 27.97 | 152.03 |

### 2.2: Automated Formulation

RALA/mRNA nanoparticles were formulated at various N:P ratios by use of an automated microfluidics system (e.g. Precision Nanosystems Ignite NanoAssemblr). Two solutions at the appropriate concentrations (Table 7) were loaded into syringes, and the syringes subsequently loaded into the microfluidics system. Nanoparticles were created using a Total Flow Rate (TFR) of 10 mL/min, and a Flow Rate Ratio (FRR) of 1:3 (mRNA:RALA). The resultant solution from the system contained nanoparticles at the concentration of 0.1 μg/μl of mRNA.

TABLE 7
Automated preparation of RALA/mRNA nanoparticles
at different N:P ratios.

| | | | Concentrations | | Volumes | |
|---|---|---|---|---|---|---|
| N:P | Masses (μg) | | mRNA | RALA | mRNA | RALA |
| ratio | mRNA | RALA | (μg/μl) | (μg/μl) | (μl) | (μl) |
| 0:1 | 20 | 0 | 0.4 | 0 | 50 | 150 |
| 1:1 | 20 | 27.97 | 0.4 | 0.19 | 50 | 150 |
| 2:1 | 20 | 55.93 | 0.4 | 0.37 | 50 | 150 |
| 3:1 | 20 | 83.9 | 0.4 | 0.56 | 50 | 150 |
| 4:1 | 20 | 111.86 | 0.4 | 0.75 | 50 | 150 |
| 5:1 | 20 | 139.83 | 0.4 | 0.93 | 50 | 150 |
| 6:1 | 20 | 167.8 | 0.4 | 1.12 | 50 | 150 |
| 7:1 | 20 | 195.76 | 0.4 | 1.31 | 50 | 150 |
| 8:1 | 20 | 223.73 | 0.4 | 1.49 | 50 | 150 |
| 9:1 | 20 | 251.7 | 0.4 | 1.68 | 50 | 150 |
| 10:1 | 20 | 279.66 | 0.4 | 1.86 | 50 | 150 |
| 11:1 | 20 | 307.63 | 0.4 | 2.05 | 50 | 150 |
| 12:1 | 20 | 335.59 | 0.4 | 2.24 | 50 | 150 |

The following nanoparticles were prepared (mRNA and trehalose:mannose ratios quoted are % w/w):

TABLE 8
Examples

| Cpd | | Uridine | Cytidine | | Poly(A) | N:P | Source of | Manual/ | |
|---|---|---|---|---|---|---|---|---|---|
| no | mRNA | Mod | Mod | 5′ Cap | length | ratio | RALA | automated | Trehalose:mannose |
| 001 | EGFP | None | None | CleanCap ® | 120 | 3:1 | Biomatik | Manual | 100:0 |
| 002 | EGFP | None | None | CleanCap ® | 120 | 4:1 | Biomatik | Manual | 100:0 |
| 003 | EGFP | None | None | CleanCap ® | 120 | 5:1 | Biomatik | Manual | 100:0 |
| 004 | EGFP | None | None | CleanCap ® | 120 | 6:1 | Biomatik | Manual | 100:0 |
| 005 | EGFP | None | None | CleanCap ® | 120 | 7:1 | Biomatik | Manual | 100:0 |
| 006 | Cy5-EGFP | None | None | CleanCap ® | 120 | 3:1 | Biomatik | Manual | 100:0 |
| 007 | Cy5-EGFP | None | None | CleanCap ® | 120 | 4:1 | Biomatik | Manual | 100:0 |
| 008 | Cy5-EGFP | None | None | CleanCap ® | 120 | 5:1 | Biomatik | Manual | 100:0 |
| 009 | Cy5-EGFP | None | None | CleanCap ® | 120 | 6:1 | Biomatik | Manual | 100:0 |
| 010 | Cy5-EGFP | None | None | CleanCap ® | 120 | 7:1 | Biomatik | Manual | 100:0 |
| 011 | mRNA (E7) | ψ | 5-methyl | ARCA | 120 | 7:1 | Biomatik | Manual | 100:0 |
| 012 | mRNA (E7) | m1ψ | 5-methyl | CleanCap ® | 120 | 7:1 | Biomatik | Manual | 100:0 |
| 013 | mRNA (E7) | m1ψ | None | CleanCap ® | 120 | 7:1 | Biomatik | Manual | 100:0 |
| 014 | mRNA (E7) | None | None | CleanCap ® | 120 | 7:1 | Biomatik | Manual | 100:0 |
| 015 | mRNA (E7) | None | None | ARCA | 120 | 7:1 | Biomatik | Manual | 100:0 |
| 016 | mRNA (E7) | m1ψ | None | CleanCap ® | 120 | 7:1 | Biomatik | Manual | 0:100 |
| 017 | mRNA (E7) | m1ψ | None | CleanCap ® | 120 | 7:1 | Biomatik | Manual | 85:15 |
| 018 | mRNA (E6) | m1ψ | None | CleanCap ® | 120 | 7:1 | Biomatik | Manual | 100:0 |
| 019 | mRNA (E7) | m1ψ | None | CleanCap ® | 120 | 7:1 | Biomatik | Manual | 85:15 |
| 020 | mRNA | m1ψ | None | CleanCap ® | 120 | 7:1 | Biomatik | Manual | 85:15 |
| | (E6):mRNA | | | | | | | | |
| | (E7) | | | | | | | | |
| | (25:75) | | | | | | | | |
| 021 | mRNA | m1ψ | None | CleanCap ® | 120 | 7:1 | Biomatik | Manual | 85:15 |
| | (E6):mRNA | | | | | | | | |
| | (E7) | | | | | | | | |
| | (50:50) | | | | | | | | |
| 022 | mRNA | m1ψ | None | CleanCap ® | 120 | 7:1 | Biomatik | Manual | 85:15 |
| | (E6):mRNA | | | | | | | | |
| | (E7) | | | | | | | | |
| | (75:25) | | | | | | | | |
| 023 | mRNA (E6) | m1ψ | None | CleanCap ® | 120 | 7:1 | Biomatik | Manual | 85:15 |
| 024 | mRNA | None | None | CleanCap ® | 120 | 7:1 | Biomatik | Manual | 85:15 |
| | (E6):mRNA | | | | | | | | |
| | (E7) | | | | | | | | |
| | (25:75) | | | | | | | | |
| 025 | mRNA (E7) | m1ψ | None | CleanCap ® | 120 | 7:1 | Biomatik | Automated | 100:0 |
| 026 | mRNA (E7) | m1ψ | None | CleanCap ® | 120 | 8:1 | Biomatik | Automated | 100:0 |
| 027 | mRNA (E7) | m1ψ | None | CleanCap ® | 120 | 9:1 | Biomatik | Automated | 100:0 |
| 028 | mRNA (E7) | m1ψ | None | CleanCap ® | 120 | 10:1 | Biomatik | Automated | 100:0 |
| 029 | mRNA (E7) | m1ψ | None | CleanCap ® | 120 | 11:1 | Biomatik | Automated | 100:0 |
| 030 | mRNA (E7) | m1ψ | None | CleanCap ® | 120 | 12:1 | Biomatik | Automated | 100:0 |
| 031 | mRNA | m1ψ | None | CleanCap ® | 120 | 9:1 | Biomatik | Automated | 100:0 |
| | (E6):mRNA | | | | | | | | |
| | (E7) | | | | | | | | |
| | (50:50) | | | | | | | | |
| 032 | mRNA | m1ψ | None | CleanCap ® | 120 | 9:1 | Biomatik | Automated | 100:0 |
| | (E6):mRNA | | | | | | | | |
| | (E7):mRNA | | | | | | | | |
| | (IL-15) | | | | | | | | |
| | (40:40:20) | | | | | | | | |
| 033 | mRNA | m1ψ | None | CleanCap ® | 120 | 7:1 | BACHEM | Automated | 100:0 |
| | (E6):mRNA | | | | | | | | |
| | (E7) | | | | | | | | |
| | (50:50) | | | | | | | | |
| 034 | mRNA | m1ψ | None | CleanCap ® | 120 | 8:1 | BACHEM | Automated | 100:0 |
| | (E6):mRNA | | | | | | | | |
| | (E7) | | | | | | | | |
| | (50:50) | | | | | | | | |
| 035 | mRNA | m1ψ | None | CleanCap ® | 120 | 9:1 | BACHEM | Automated | 100:0 |
| | (E6):mRNA | | | | | | | | |
| | (E7) | | | | | | | | |
| | (50:50) | | | | | | | | |
| 036 | mRNA | m1ψ | None | CleanCap ® | 120 | 10:1 | BACHEM | Automated | 100:0 |
| | (E6):mRNA | | | | | | | | |
| | (E7) | | | | | | | | |
| | (50:50) | | | | | | | | |
| 037 | mRNA | m1ψ | None | CleanCap ® | 120 | 11:1 | BACHEM | Automated | 100:0 |
| | (E6):mRNA | | | | | | | | |
| | (E7) | | | | | | | | |
| | (50:50) | | | | | | | | |
| 038 | mRNA | m1ψ | None | CleanCap ® | 120 | 12:1 | BACHEM | Automated | 100:0 |
| | (E6):mRNA | | | | | | | | |
| | (E7) | | | | | | | | |
| | (50:50) | | | | | | | | |
| 039 | mRNA | m1ψ | None | CleanCap ® | 120 | 9:1 | BACHEM | Automated | 100:0 |
| | (E6):mRNA | | | | | | | | |
| | (E7):mRNA | | | | | | | | |
| | (E1) | | | | | | | | |
| | (33.3:33.3:33.3) | | | | | | | | |
| 040 | mRNA | m1ψ | None | CleanCap ® | 80 | 9:1 | BACHEM | Automated | 100:0 |
| | (E6):mRNA | | | | | | | | |
| | (E7) | | | | | | | | |
| | (50:50) | | | | | | | | |
| 041 | Cy5-EGFP | None | None | CleanCap ® | 120 | 9:1 | BACHEM | Automated | 100:0 |

### Example 3: Lyophilisation of RALA/mRNA Nanoparticles

200 μL of RALA/mRNA nanoparticles prepared as in Example 2 was transferred into a 2 mL lyophilisation vial. As cryoprotectants, trehalose (20% w/v), mannose (20% w/v) or a blend of trehalose and mannose (85:15, final 20% w/v) were added to the nanoparticle solution in the vial, such that the volume added was equal to half the final reconstitution volume ensuring that once reconstituted in water following lyophilisation, the resultant solution contained 10% w/v cryoprotectant. Following the addition of cryoprotectant, a rubber lyophilisation vial stopper was partially placed on the vial, such that the air-flow notches were still functional to facilitate sublimation during the lyophilisation process. Vials were subsequently loaded into a programmable freeze dryer (e.g. SP Scientific AdVantage Pro) according to the following lyophilisation procedure (Table 9).

TABLE 9
Lyophilisation Procedure

| 1 | Thermal Treatment | | | | |
|---|---|---|---|---|---|
| | | Shelf Temp. | Ramp Time | Hold Time | |
| | Step | [° C.] | [min] | [min] | |
| | 1 | 5 | 60 | 10 | |
| | 2 | −35 | 90 | 0 | |
| 2 | Drying Steps | | | | |
| | | Shelf Temp. | Ramp Time | Hold Time | Vacuum |
| | Step | [° C.] | [min] | [min] | [mTorr] |
| | 3 | −35 | 0 | 180 | 120 |
| | 4 | −30 | 60 | 0 | 190 |
| | 5 | −30 | 0 | 180 | 190 |
| | 6 | −25 | 60 | 0 | 190 |
| | 7 | −25 | 0 | 180 | 190 |
| | 8 | 20 | 120 | 0 | 190 |
| | 9 | 20 | 0 | 360 | 190 |
| 3 | Storage | | | | |
| | Shelf Temp | 20 | ° C. | | |
| | Vacuum | 50 | mTorr | | |

| 4 | Backfilling | None |
|---|---|---|
| 5 | Stoppering | Vials Stoppered under Vacuum |

### Example 4: Nanoparticle Size and Charge Analysis

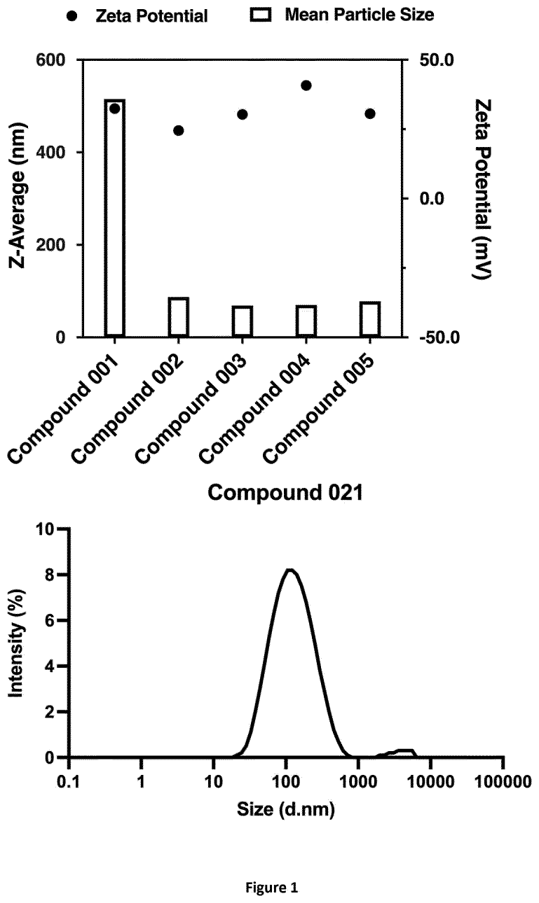

Z-Average particle size measurements and polydispersity (Pdl) of RALA/mRNA nanoparticles prepared as in Example 2, were performed using Dynamic Light Scattering (DLS) in order to obtain particle size and charge distributions. Surface charge measurements of the RALA nanoparticles were determined by Laser Doppler Velocimetry. The zeta potential of the particles was measured using disposable foldable zeta cuvettes. Zeta cuvettes for the measurement of zeta potential were first washed with 70% ethanol, followed by two rinses with double distilled H₂O prior to loading the sample. Lyophilised sample was reconstituted by the addition of 50 μl of ultrapure water. The undiluted sample was used for size measurements, subsequently diluted to 1 mL with ultrapure water and then 700-800 μL of diluted sample was used for determination of zeta potential. The nanoparticles were made up at a range of N:P ratios using at least 1 μg of mRNA in each sample. Nanoparticles were analysed on a Zetasizer-Nano-ZS (Malvern Instruments) with DTS software (Malvern Instruments, UK) and exemplar results for compounds 001, 002, 003, 004, 005 are shown in FIG. 1. FIG. 1 also contains an example size distribution plot of compound 021 nanoparticles.

### Example 5: Tracking of Manually Formulated RALA/Cy5 mRNA Nanoparticles In Vivo

Nanoparticles were lyophilised according to Example 3 and then reconstituted in ultrapure water (0.5 mg mRNA/ml) prior to administration.

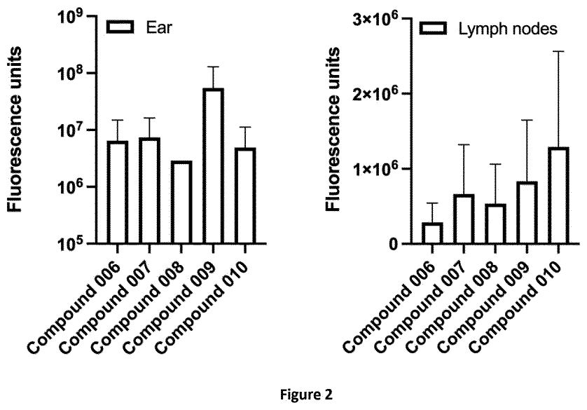

Nanoparticle tracking analysis was performed following intradermal administration of RALA/Cy5-EGFP mRNA nanoparticles (Compounds 006, 007, 008, 009 or 010) in female C57BL/6 mice of 5-6 weeks old. Mice were anaesthetised by intraperitoneal administration of ketamine HCl (Ketaset) and xylazine (Rompun), and were administered Compounds 006, 007, 008, 009 or 010 nanoparticles, equivalent to 10 μg mRNA, via intradermal injection to the ear pinna. Mice were sacrificed at the indicated timepoint, and fluorescence at site of administration and in the skin draining lymph nodes was determined by imaging using a Bruker In Vivo Xtreme imaging system (FIG. 2).

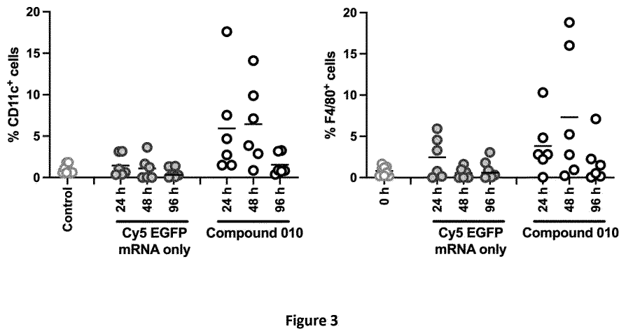

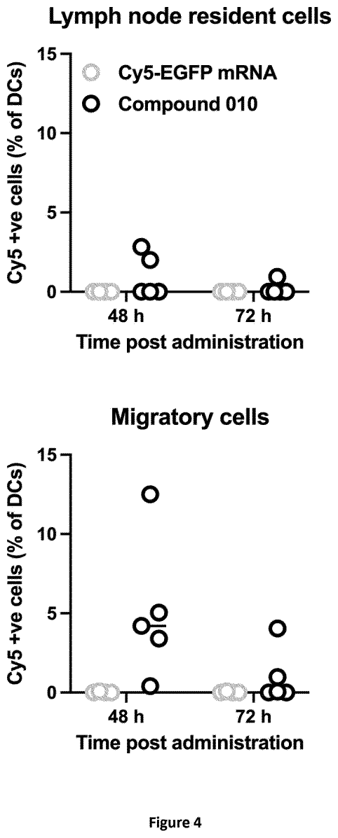

The lymph nodes were dissected from the mice, disaggregated by mechanically passing through a 70 μm cell strainer, and cells were stained for dendritic cell and macrophage markers. Dendritic cells (CD11c+) and macrophages (F480) (both antigen presenting cells) were found to be associated with the Cy5-mRNA ex vivo, and the dendritic cells that had taken up the RALA/Cy5-mRNA were migratory, suggesting that the cells which had actively taken up the nanoparticles had migrated to the lymph nodes (FIG. 3 & FIG. 4).

The nanoparticles used in the following experiments were lyophilised according to Example 3 and then reconstituted in ultrapure water (1 mg mRNA/ml) prior to administration.

### Example 6: Assessment of Immunogenicity of Manually Formulated RALA/E7 mRNA

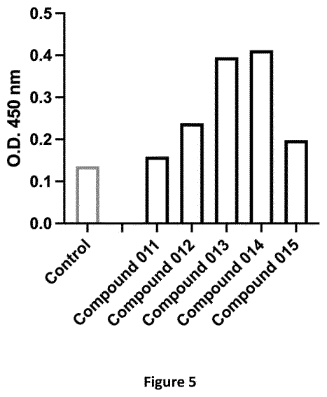

Initial studies were undertaken to determine the suite of nucleotide modifications for provoking E7 protein expression and immunogenicity. Compounds 011, 012, 013, 014 and 015 were assessed for their ability to provoke E7 expression in NCTC-929 mouse fibroblasts by in-cell ELISA. Cells plated in 96 well plates were transfected with RALA/E7 mRNA, and assessed for E7 expression 24 h later. Following fixation and permeabilization, cells were probed with an anti-HPV 16 E7 primary antibody (raised in rabbit, Abcam), excess antibody was removed by washing with PBS/0.05% Tween® 20, and cells were probed with an anti-rabbit secondary antibody conjugated to horseradish peroxidase. E7 expression was detected using TMB and Stop solution (standard ELISA reagents), and optical density at 450 nm was measured using a Molecular Devices SpectraMax® iD3 plate reader (FIG. 5).

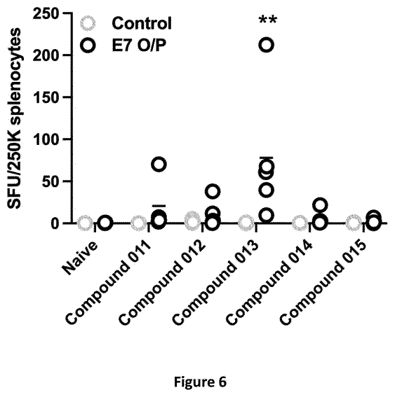

C57BL/6 mice were injected intradermally with 20 μg of Compounds 011, 012, 013, 014 and 015 at Days 0 and 21 with sacrifice at Day 42. Spleens were extracted, and single cell splenocyte suspensions were seeded into FluoroSpot® plates (Mabtech), and either stimulated with E7 overlapping peptides (Miltenyi Biotec), or with culture medium alone. FluoroSpot® analysis revealed that only Compound 013 produced high quantities of IFNγ (**p<0.01 compared with naïve, Kruskal-Wallis test with Dunn's multiple comparisons test; FIG. 6).

### Example 7: Impact of Cryoprotectant on Immunogenicity of Manually Formulated RALA/E7 mRNA

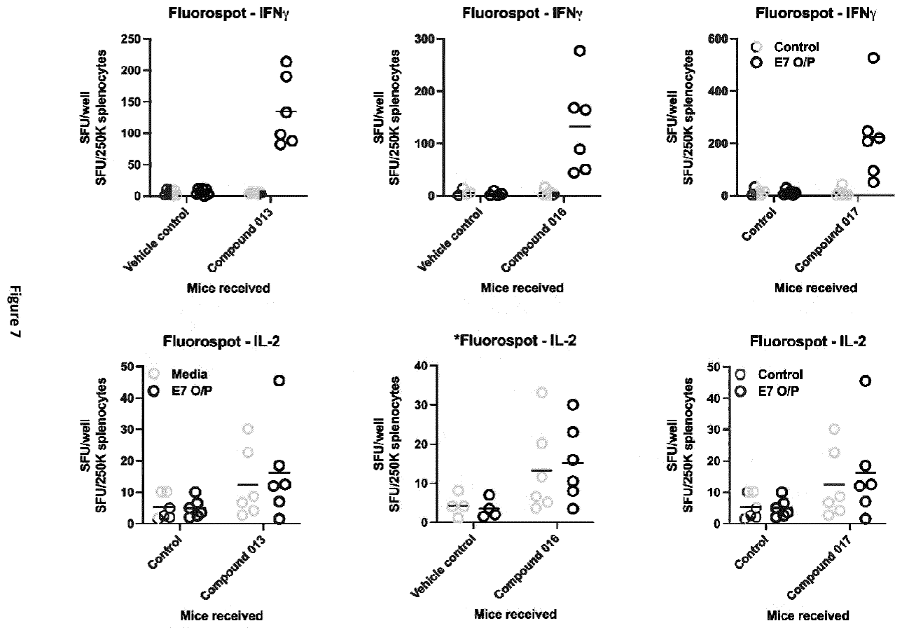

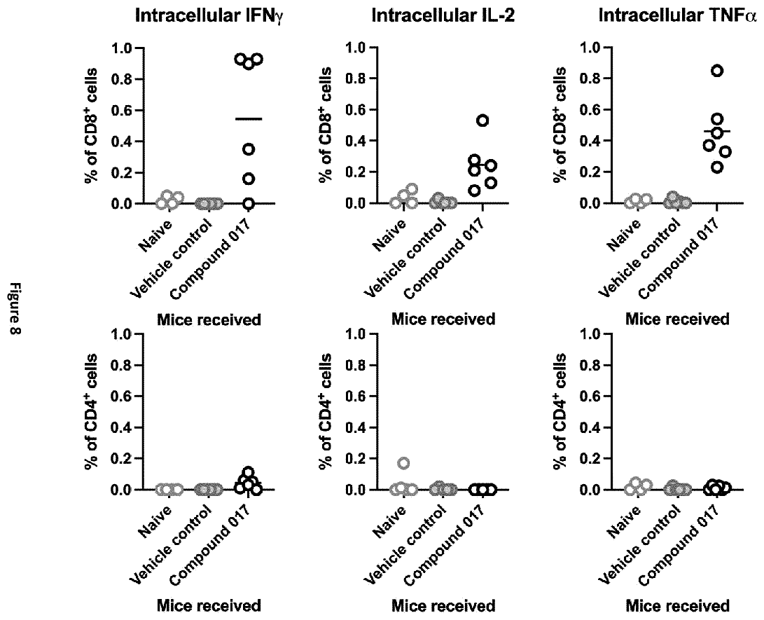

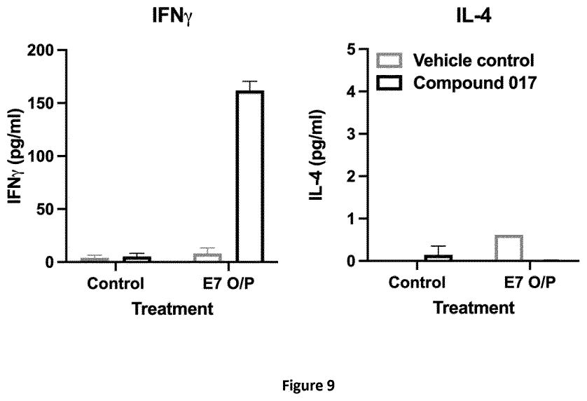

The impact of cryoprotectant on immunogenicity of Compounds 013, 016 and 017 was assessed using nanoparticles that were lyophilised using either trehalose, mannose or a 85:15 blend of trehalose:mannose as cryoprotectants. Assay and regimen were as employed in Example 6. An 85:15 blend of trehalose:mannose produced a marginally more potent response in terms of IFNγ and IL-2 production by splenocytes from Compound 017-vaccinated mice stimulated with E7 overlapping peptides (FIG. 7). Further analysis of splenocytes from these mice was conducted by flow cytometry. Cells stimulated with E7 overlapping peptides (in the presence of Brefeldin A, to prevent secretion of cytokines by stimulated cells) were stained for viability, CD3, CD4, CD8, IFNγ, IL-2 and TNFα. Viable T cells were gated depending on their CD4/CD8 expression status, and assessed for their IFNγ, IL-2 and TNFα content using a FACSCanto II flow cytometer (Becton Dickinson) (FIG. 8). Flow cytometry revealed that the source of the IFNγ and IL-2 in FluoroSpot® analysis was CD8+ cytotoxic T cells. Further analysis was performed on splenocytes that were cultured with E7 overlapping peptides, but in the absence of Brefeldin A, meaning that cytokines produced as a result of stimulation were secreted into culture medium. Analysis of IFNγ (characteristic of Th1 immune response) and interleukin 4 (IL-4-characteristic of Th2 response) by ELISA (both Abcam) revealed a strong bias towards a Th1 response in RALA/E7 mRNA-vaccinated mice (FIG. 9).

### Example 8: Assessment of Immunogenicity of Manually Formulated RALA/E6 mRNA

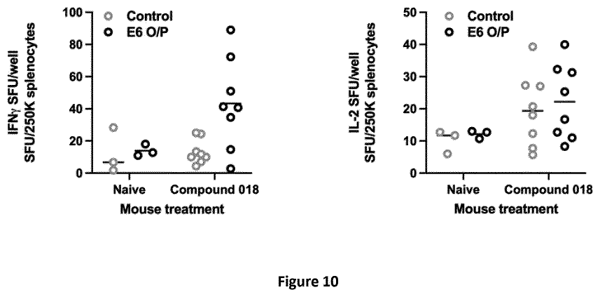

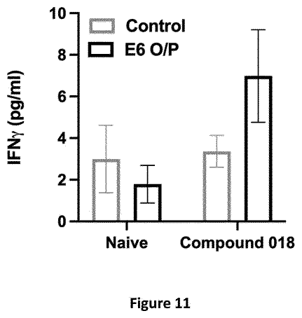

C57BL/6 mice were injected intradermally with 20 μg of Compound 018 at Days 0 and 21 with sacrifice at Day 42. Spleens were extracted, and single cell splenocyte suspensions were seeded into FluoroSpot® plates (Mabtech®), and either stimulated with E6 overlapping peptides (Miltenyi Biotec), or with culture medium alone. Stimulation of splenocytes from vaccinated mice produced IFNγ and IL-2 (FIG. 10), although less potently than the splenocytes from mice vaccinated with RALA/E7 mRNA and stimulated with E7 peptides. This is supported by the literature, which states that HPV 16 E6 does not produce strong epitopes in mice. Splenocytes from vaccinated mice that were stimulated with E6 overlapping peptides also secreted IFNγ into culture medium, as assessed by ELISA (FIG. 11).

### Example 9: Assessment of the Prophylactic and Therapeutic Benefit of Compounds 019, 020, 021, 022, 023 and 024 in TC-1 Tumour Challenge Studies

Two studies were designed to determine the potency of the vaccines in therapeutic and prophylactic models of HPV 16-related cancer. The TC-1 cell line was derived from primary lung epithelial cells of C57BL/6 mice. The cells were immortalized with the amphotropic retrovirus vector LXSN16E6E7 obtained from and subsequently transformed with the pVEJB plasmid expressing the activated human c-Ha-ras oncogene. The cell hence expresses HPV 16 E6 and E7, and is a model for HPV 16-associated malignancies.

In the prophylactic model, female C57BL/6 mice were vaccinated (two dose regimen with 3-week interval) with Compounds 019, 020, 021, 022, 023 and 024. On Day 28, mice were implanted on the flank with 5×10⁴ TC-1 cells, and tumour development was monitored.

In the therapeutic model, on Day 0, female C57BL/6 mice were implanted on the flank with 5×10⁴ TC-1 cells, and received Compound 019, 020, 021, 022, 023 or 024 on Days 8, 15 (all mice) and 43 (mice in Compound 019, 020, 021 and 022 groups). Tumour development was monitored.

On Day 108, mice in both prophylactic and therapeutic studies that lacked tumours were re-challenged with TC-1, and an age-matched naïve control group was added to the study.

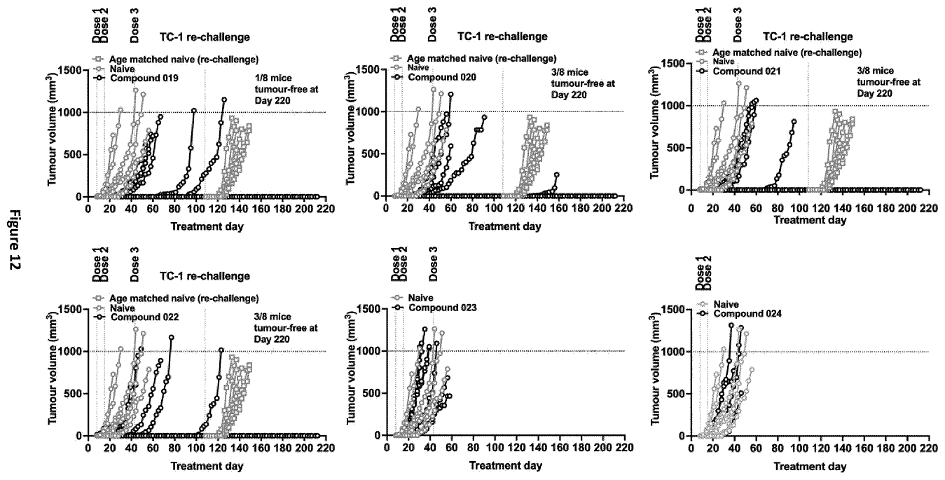

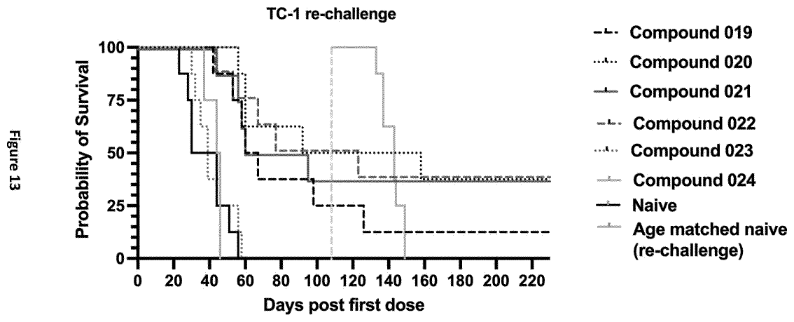

Therapeutic vaccination with Compound 019, 020, 021 and 022 slowed the development of TC-1 xenografts, while Compound 023 lacked therapeutic benefit. This is unsurprising given the relative lack of immunogenicity of Compound 018 (also comprises RALA with E6 mRNA) in the mouse immunogenicity model (FIG. 10). The majority of the mice that were tumour-free on Day 108 when mice were re-challenged with TC-1 remained tumour free until day 196 (FIG. 12 & FIG. 13).

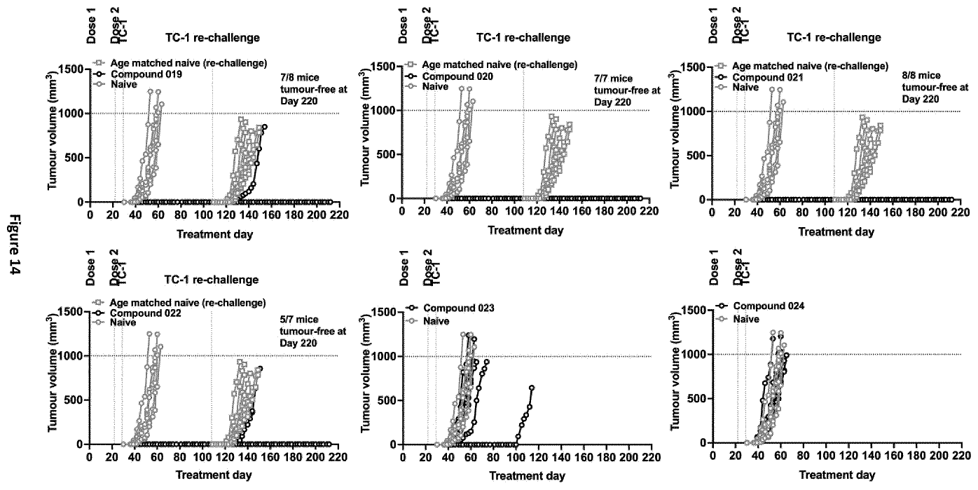

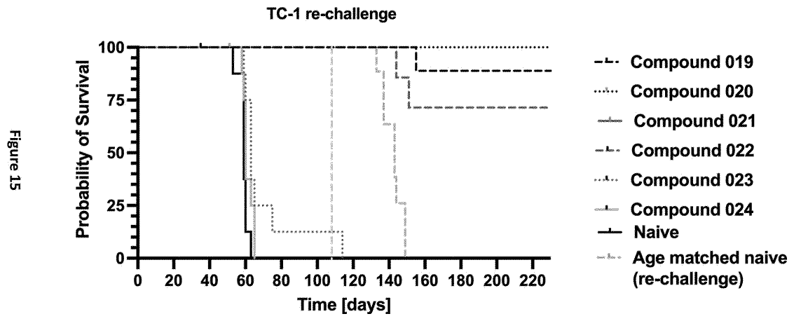

Prophylactic vaccination with Compound 019, 020, 021 and 022 prevented the engraftment of TC-1 xenografts in all mice in their respective groups. Again, Compound 023 failed to provide protection against TC-1 tumour development, as did Compound 024. Of 30 mice re-challenged with TC-1 on Day 108, only two developed tumours (FIG. 14 & FIG. 15).

### Example 10: Scaling Up Manual Formulation of RALA/E7 mRNA Nanoparticles to Automated Formulation Process

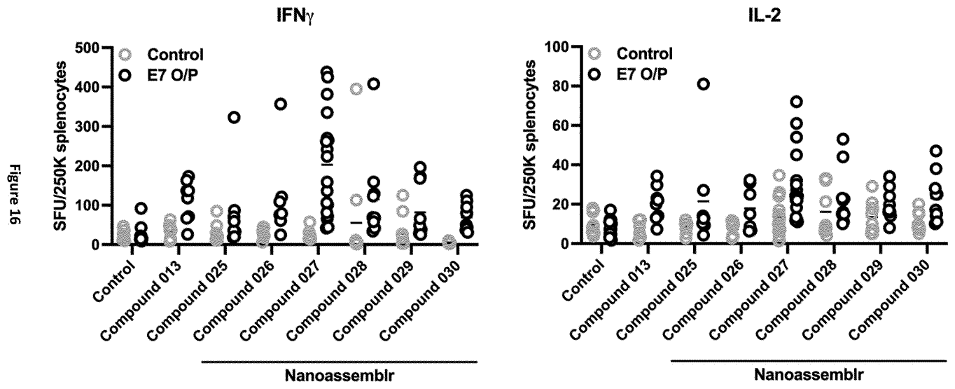

The nanoparticle formulation process has been transferred to a benchtop automated microfluidics system, where flow rates and flow rate ratios are carefully controlled, and mixing within the microfluidics cartridge is controlled by cartridge geometry. An immunogenicity study similar to that described in Example 6 was undertaken to determine the response to vaccination with Compounds 025, 026, 027, 028, 029 and 030. Compound 027 provoked the strongest E7-specific response in terms of secretion of IFNγ and IL-2 in FluoroSpot® analysis (FIG. 16).

### Example 11: Incorporation of IL-15 mRNA into Formulation Provokes a Stronger Memory Phenotype in T Cells

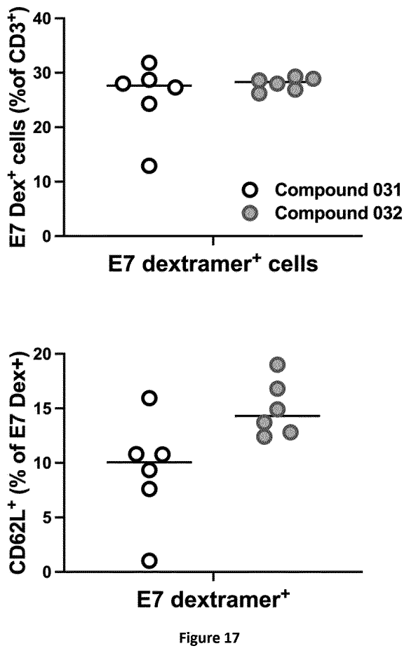

C57BL/6 mice were injected intradermally with Compounds 031 or 032 at Days 0, 4 and 7 with sacrifice at Day 28. Spleens were extracted, and single cell splenocyte suspensions were stained with an E7-specific dextramer, which will bind E7-specific CD8+ T cells. Cells were also stained for CD3 (pan T cell marker) and CD62L (presence of which distinguishes between T cells of an effector phenotype (CD62L) and a central memory phenotype (CD62L+)). Although IL-15's presence did not impact the number of T cells that were positive for the E7 dextramer, inclusion of IL-15 mRNA in Compound 032 increased the number of T cells with a central memory phenotype, compared with Compound 031, which lacks IL-15 (FIG. 17).

### Example 12: Assessment of the Optimal Dose for Therapeutic Benefit of Compounds 031 in TC-1 Tumour Challenge Studies

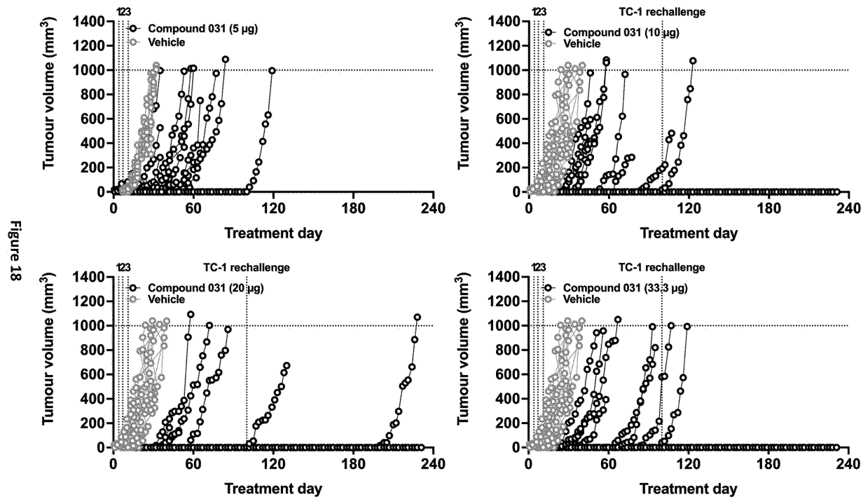

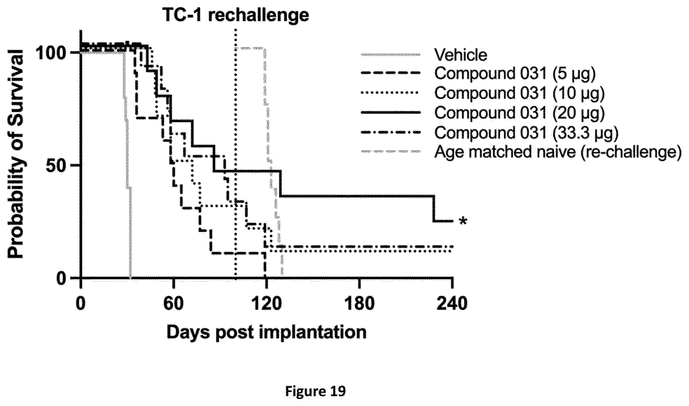

Female C57BL/6 mice (5-6 weeks old) were inoculated with 1×10⁵ TC-1 cells subcutaneously onto the flank with a 23-gauge needle. Tumour volume was monitored for the following 245 days. On study days 4, 7, and 11, all mice were vaccinated intradermally into the left ear with Compound 031. A maximum tumour volume threshold of 1000 mm³ was set for the study (FIG. 18). Mice that were tumour-free at Day 100 were re-challenged with 1×10⁵ TC-1 cells. Maximum efficacy was observed at 20 μg Compound 031, with 3/9 mice tumour-free at endpoint. 1/10 mice remained tumour-free in the 10 μg and 33.3 μg groups. Vertical dotted lines labelled 1, 2 and 3 signify treatment days. All doses of Compound 031 improved survival of tumour-challenged mice, with only 20 μg improving survival significantly above that observed with 5 μg Compound 031 (*p<0.05, Logrank Mantel-Cox test; FIG. 19).

### Example 13: Assessment of the Immunogenicity of Compound 031 Compared with Compound 033, Formulated Using RALA from Bachem

This study was undertaken to determine the equivalence between Bachem® and Biomatik® RALA peptide suppliers.

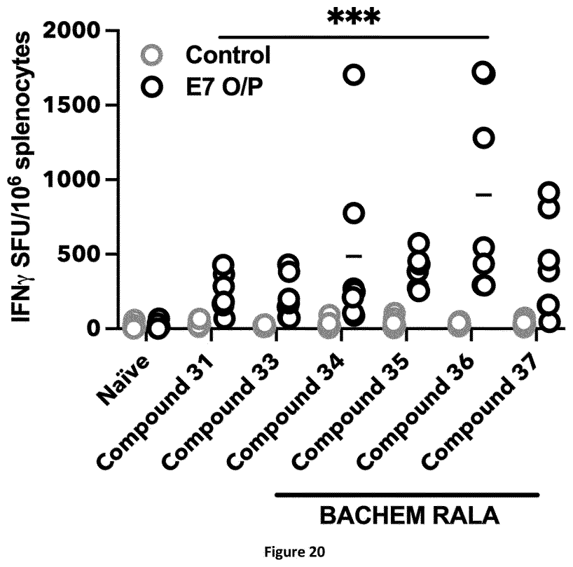

C57BL/6 mice were injected intradermally with 20 μg of Compound 031 (Biomatik® RALA) or Compound 033, 034, 035, 036 or 037 at Days 0 and 21 with sacrifice at Day 42. Spleens were extracted, and single cell splenocyte suspensions were seeded into ELISpot plates (Mabtech®), and stimulated with E7 overlapping peptides (Jerini Peptide Technologies®), or with culture medium alone. Vaccination of mice with all Compounds resulted in IFNγ-responsive cells in the spleens at Day 42 (***p<0.001, FIG. 20).

### Example 14: Assessment of the Immunogenicity of Compound 035 when Administered Via Intradermal, Intravenous, and Intramuscular Routes of Administration

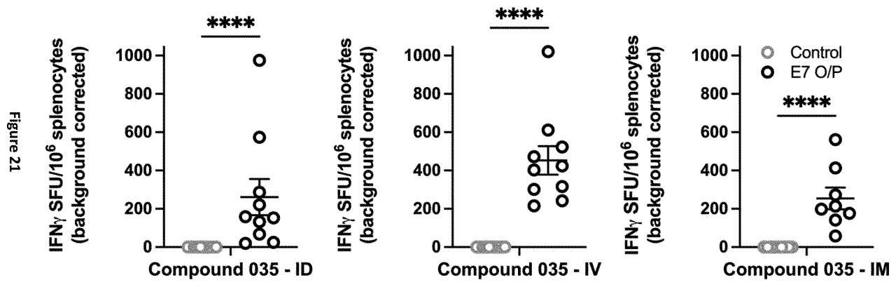

Female C57BL/6 mice (5-6 weeks) received 20 μg/dose of Compound 035 in a volume of 20 μl (ID & IM) or 100 μl (IV), and were treated on Days 0 and 21. On Day 42, mice were culled by carbon dioxide asphyxiation, the spleen was aseptically resected from each mouse and placed in MACS Tissue Storage Solution for downstream processing. Spleens were extracted, and single cell splenocyte suspensions were seeded into ELISpot plates (Mabtech®), and stimulated with E7 overlapping peptides (Jerini Peptide Technologies®), or with culture medium alone. Vaccination via all routes of administration resulted in the accumulation of E7-responsive cells in the spleens of mice, as determined by IFNγ ELISpot (****p<0.001 compared with control; Mann-Whitney test; FIG. 21).

### Example 15: Assessment of the Therapeutic Benefit of Intravenous or Intradermal Administration of Compound 035 in TC-1 Tumour Challenge Studies

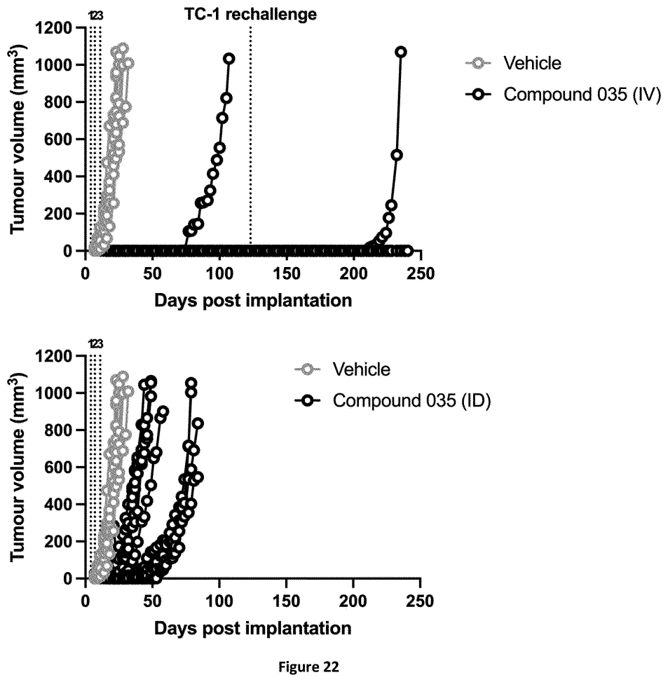

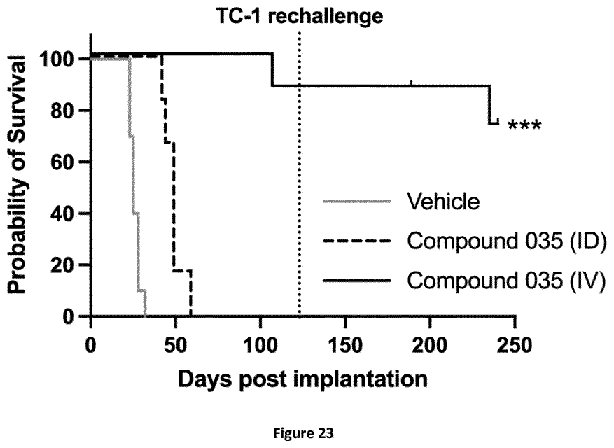

Female C57BL/6 mice (5-6 weeks old) were inoculated with 1×10⁵ TC-1 cells subcutaneously onto the flank with a 23-gauge needle. Tumour volume was monitored for the following 240 days. On study days 4, 7, and 11, all mice were vaccinated intravenously (lateral tail vein) or intradermally (left ear) with Compound 035. A maximum tumour volume threshold of 1000 mm³ was set for the study. Treatment with Compound 035 via either route slowed tumour development, with greater efficacy observed in the intravenous group (FIG. 22). Mice that were tumour-free at Day 123 were re-challenged with 1×10⁵ TC-1 cells. Maximum efficacy was observed in the IV group, with 5/7 mice tumour-free at endpoint. 0/10 mice remained tumour-free in the ID group. Vertical dotted lines labelled 1, 2 and 3 signify treatment days. Treatment with Compound 035 via either route also significantly improved survival in tumour-challenged mice. Importantly, survival in intravenously-treated mice was significantly improved compared to intradermally-treated mice (***p<0.01 compared with vehicle; Logrank Mantel-Cox test; FIG. 23).

### Example 16: Additional Assessment of the Therapeutic Benefit of Intravenous or Intradermal Administration of Compound 035 in TC-1 Tumour Challenge Studies

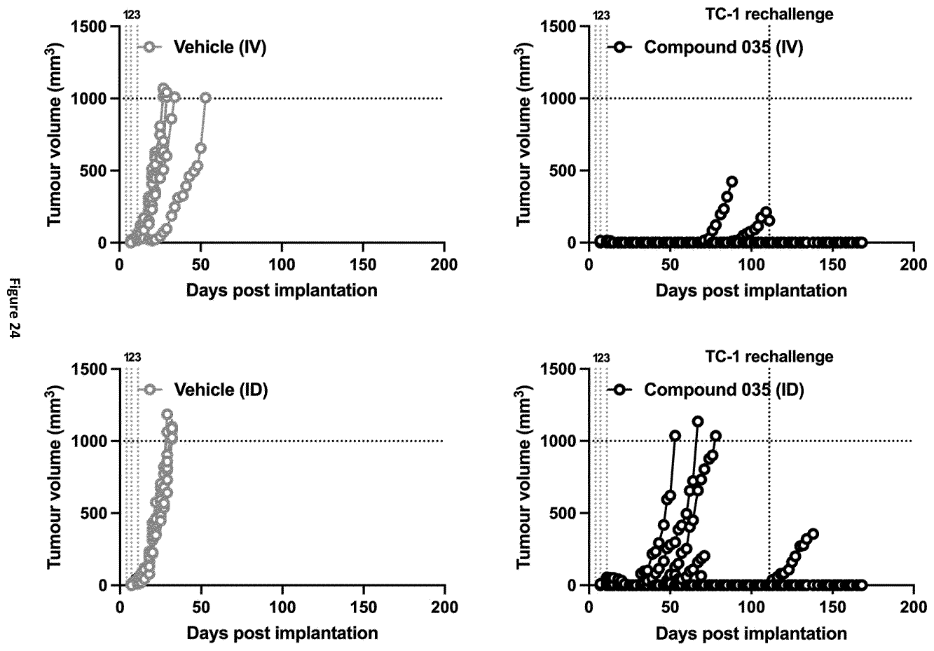

Female C57BL/6 mice (5-6 weeks old) were inoculated with 1×10⁵ TC-1 cells subcutaneously onto the flank with a 23-gauge needle. Tumour volume has been monitored to Day 168 (study is still ongoing). On study days 4, 7, and 11, all mice were vaccinated intravenously (lateral tail vein) or intradermally (left ear) with Compound 035. A maximum tumour volume threshold of 1000 mm³ was set for the study. Treatment with Compound 035 via either route slowed tumour development, with greater efficacy observed in the intravenous group (FIG. 24). Mice that were tumour-free at Day 111 were re-challenged with 1×10⁵ TC-1 cells. Mice that were tumour-free at Day 111 were re-challenged with 1×10⁵ TC-1 cells. 7/9 mice in the IV group, and 4/10 mice in the ID group are tumour-free at present. Vertical dotted lines labelled 1, 2 and 3 signify treatment days.

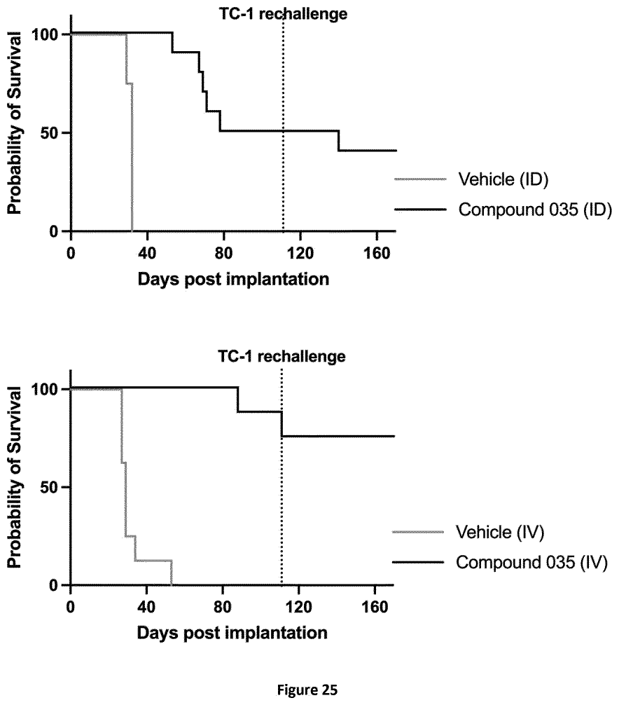

Treatment with Compound 035 via either route also improved survival in tumour-challenged mice (FIG. 25). As this study is still ongoing, statistical analysis of survival proportions is not appropriate.

### Example 17: Assessment of the Therapeutic Benefit of Intravenous or Intradermal Administration of Compound 035 and Compound 039 in C3.43 Tumour Challenge Studies

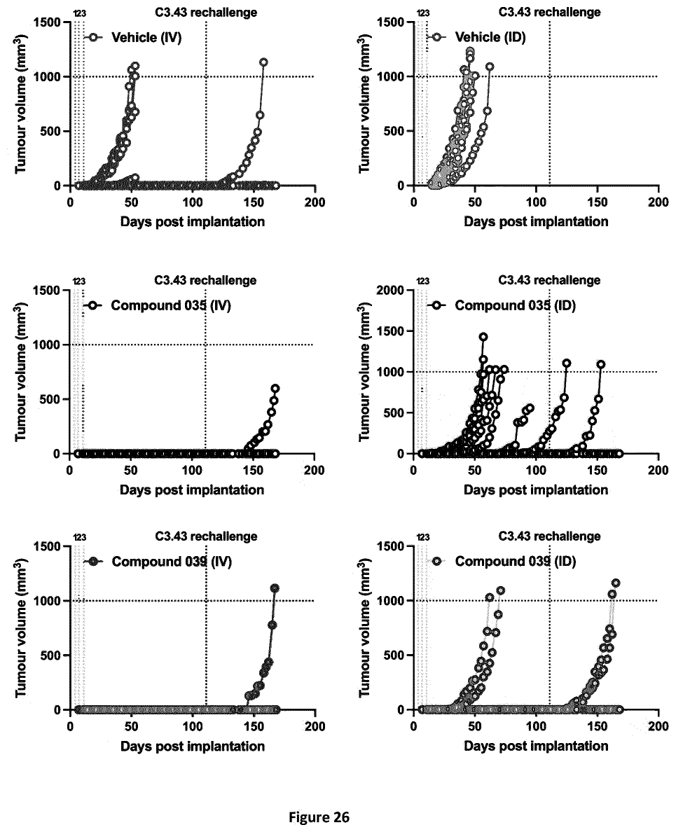

Female C57BL/6 mice (5-6 weeks old) were inoculated with 1×10⁵ C3.43 cells subcutaneously onto the flank with a 23-gauge needle. Tumour volume has been monitored to Day 168 (study is still ongoing). On study days 4, 7, and 11, all mice were vaccinated intravenously (lateral tail vein) or intradermally (left ear) with Compound 035 or Compound 039. A maximum tumour volume threshold of 1000 mm³ was set for the study. Treatment with Compound 035 or 039 via either route slowed tumour development, with greater efficacy observed in the intravenous group (FIG. 26). Mice that were tumour-free at Day 111 were re-challenged with 1×10⁵ C3.43 cells. Mice that were tumour-free at Day 111 were re-challenged with 1×10⁵ C3.43 cells. 9/10 mice (Compound 035) and 6/7 (Compound 039) in the IV group, and 1/10 mice (Compound 035) and 3/8 (Compound 039) in the ID group are tumour-free at present. Vertical dotted lines labelled 1, 2 and 3 signify treatment days.

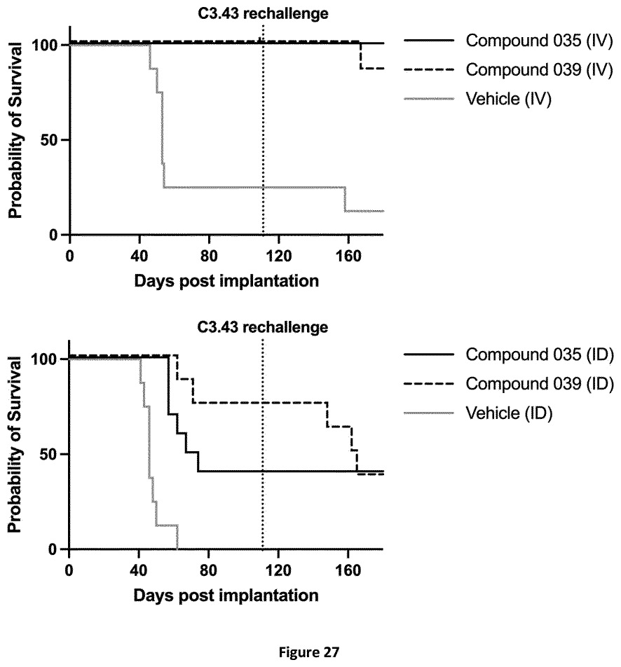

Treatment with Compound 035 or Compound 039 via either route also improved survival in tumour-challenged mice (FIG. 27). As this study is still ongoing, statistical analysis of survival proportions is not appropriate.

### Example 18: Assessment of the Immunogenicity of RALA Vaccine Nanoparticles Comprising mRNAs with 80 Nucleotide Long Poly(A) Tail

This study was undertaken to determine the equivalence between 120 nucleotide long poly(A) tailed mRNA and 80 nucleotide poly(A) tailed mRNA.

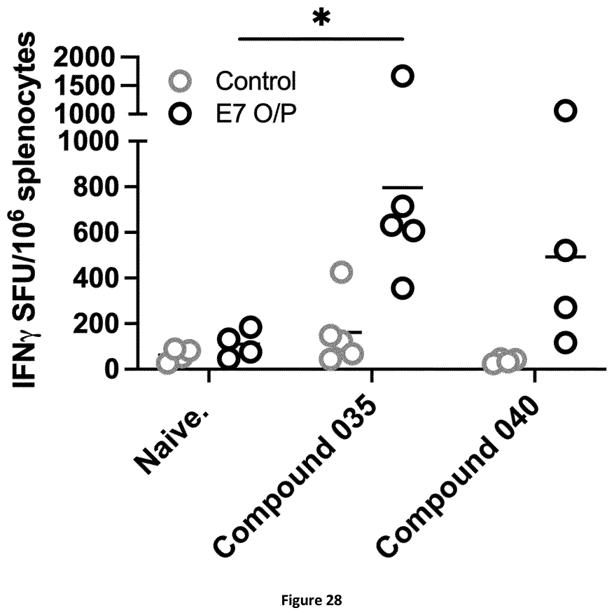

C57BL/6 mice were injected intradermally with 20 μg of Compound 035 (120 poly(A)) or Compound 040 (80 poly(A)) at Days 0 and 21 with sacrifice at Day 42. Spleens were extracted, and single cell splenocyte suspensions were seeded into ELISpot plates (Mabtech®), and stimulated with E7 overlapping peptides (Jerini Peptide Technologies®), or with culture medium alone. Vaccination of mice with Compounds 035 or 040 resulted in accumulation of E7 antigen-responsive cells (as demonstrated using IFNγ ELISpot) in the spleens at Day 42 (FIG. 28). *p<0.01 compared to naïve (Kruskal-Wallis test with Dunn's multiple comparisons test). There was no significant difference in the response between Compound 035 and Compound 040.

### Example 19: Assessment of the Stability of Compound 035 at 4° C. and at 25° C., 60% Relative Humidity

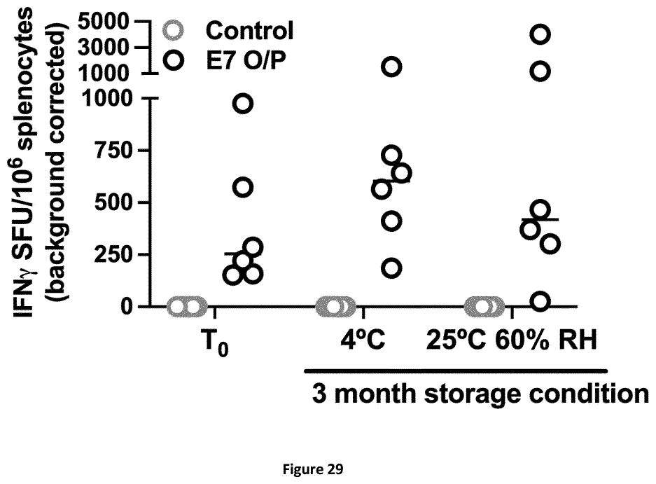

Compound 035 was formulated and stored for a period of three months at either 4° C. (refrigerator) or at 25° C., 60% relative humidity (stability cabinet with controlled temperature and humidity). Following three months' storage, formulations were reconstituted. C57BL/6 mice were injected intradermally with 20 μg of Compound 035 (120 poly(A)) or Compound 040 (80 poly(A)) at Days 0 and 21 with sacrifice at Day 42. Spleens were extracted, and single cell splenocyte suspensions were seeded into ELISpot plates (Mabtech®), and stimulated with E7 overlapping peptides (Jerini Peptide Technologies®), or with culture medium alone. Vaccination of mice with Compounds 035 or 040 resulted in accumulation of E7 antigen-responsive cells (as demonstrated using IFNγ ELISpot) in the spleens at Day 42. There was no significant difference in the response between Compound 035 that was freshly formulated (To), and the Compound 035 that was stored at either 4° C. or at 25° C., 60% relative humidity (Kruskal-Wallis test with Dunn's multiple comparisons test; FIG. 29).

### Example 20: Assessment of Antigen-Presenting Cell Uptake of Compound 041 Following Intravenous Administration

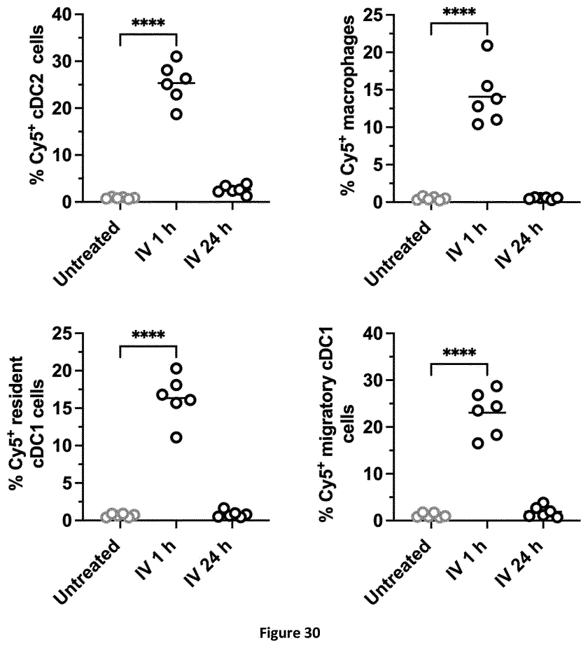

Female C57BL/6 mice (5-6 weeks old) were treated intravenously Compound 041 (10 μg), and at 1 hour post-treatment or 24 hours post-treatment, mice were sacrificed, spleens isolated and processed to single cell suspensions. To facilitate dendritic cell and macrophage differentiation, cells were stained using fluorescently conjugated antibodies (CD8-Thermo Fisher_CD8a Monoclonal Antibody (53-6.7), Super Bright™ 600_[63-0081-82]; CD4-ThermoFisher_CD4 Monoclonal Antibody (GK1.5), Super Bright™ 702_[67-0041-82]; cD11c-ThermoFisher_CD11c Monoclonal Antibody (N418), PE-Cyanine7_[25-0114-82]; cD11b-ThermoFisher_CD11b Monoclonal Antibody (M1/70), PE-eFluor™ 610_[61-0112-82]; F4/80 ThermoFisher_F4/80 Monoclonal Antibody (BM8), PerCP-Cyanine5.5_[45-4801-82]; CD103-Thermo Fisher_CD103 Monoclonal Antibody (2E7), PE_[12-1031-82]; Viability-eBioscience™ Fixable Viability Dye eFluor™ 450_[65-0863-14]). Cells that had taken up Compound 041 were positive for the Cy5 signal. There was significant uptake of Compound 041 by dendritic cells and macrophages at 1 hour post-treatment (Two-way ANOVA with Dunnett's multiple comparisons test ****p<0.0001 compared to naïve mice; FIG. 30).

### Statements

Statement 1. A vaccine, comprising:
i) an mRNA polynucleotide comprising an open reading frame encoding an antigen from an infectious microorganism or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

Statement 2. A vaccine as stated in statement 1 further comprising a second mRNA polynucleotide comprising an open reading frame encoding an antigen from an infectious microorganism or an immunogenic fragment thereof.

Statement 3. A vaccine as stated in statement 2 wherein the two mRNA polynucleotides are present in about a 50:50% w/w ratio.

Statement 4. A vaccine as stated in any one of the preceding statements wherein the open reading frame encodes an HPV antigen or an immunogenic fragment thereof.

Statement 5. A vaccine as stated in any one of the preceding statements wherein the open reading frame encodes an E6 or an E7 HPV antigen or an immunogenic fragment thereof.

Statement 6. A vaccine as stated in any one of the preceding statements wherein the open reading frame encodes an HPV 6, 8, 11, 16 or 18 antigen or an immunogenic fragment thereof.

Statement 7. A vaccine as stated in any one of the preceding statements wherein the open reading frame encodes an HPV 16 antigen or an immunogenic fragment thereof.

Statement 8. A vaccine as stated in any one of the preceding statements wherein the amphipathic cell penetrating peptide comprises the amino acid sequence WEARLARALARALARHLARALARALRACEA (SEQ ID_No 1).

Statement 9. A vaccine as stated in any one of statements 1-3 comprising:
i) a first mRNA polynucleotide comprising an open reading frame comprising a sequence selected from SEQ ID_No 2;
ii) a second mRNA polynucleotide comprising an open reading frame comprising a sequence selected from SEQ ID_No 3;
iii) an amphipathic cell penetrating peptide comprising the amino acid sequence WEARLARALARALARHLARALARALRACEA (SEQ ID_No 1).

Statement 10. A vaccine as stated in any one of statements 1-3 comprising:
i) a first mRNA polynucleotide comprising an open reading frame encoding an antigen selected from SEQ ID_No 5;
ii) a second mRNA polynucleotide comprising an open reading frame encoding an antigen selected from SEQ ID_No 6;
iii) an amphipathic cell penetrating peptide comprising the amino acid sequence

(SEQ ID_No 1)
WEARLARALARALARHLARALARALRACEA.

Statement 11. A vaccine as stated in any one of the preceding statements wherein the vaccine further comprises an mRNA polynucleotide comprising an open reading frame encoding an immunoadjuvant.

Statement 12. A vaccine as stated in statement 11 wherein the mRNA polynucleotide comprising an open reading frame encoding an immunoadjuvant encodes a cytokine.

Statement 13. A vaccine as stated in statement 11 or statement 12 wherein the mRNA polynucleotide comprising an open reading frame encoding an immunoadjuvant comprises a sequence selected from SEQ ID_No 8.

Statement 14. A vaccine as stated in statement 11 or statement 12 wherein the mRNA polynucleotide comprising an open reading frame encoding an immunoadjuvant encodes an immunoadjuvant selected from SEQ ID_No 10.

Statement 15. A vaccine as stated in any one of the preceding statements wherein the mRNA polynucleotide comprises a five-prime cap (5′ cap).

Statement 16. A vaccine as stated in any one of the preceding statements wherein the mRNA polynucleotide comprises modified uridine nucleotides wherein all uridine nucleotides are N1-methylpseudouridine nucleotides.

Statement 17. A vaccine as stated in any one of the preceding statements wherein the mRNA polynucleotide comprises unmodified cytidine nucleotides.

Statement 18. A vaccine as stated in any one of the preceding statements wherein the amphipathic cell penetrating peptide consists of the amino acid sequence WEARLARALARALARHLARALARALRACEA (SEQ ID_No 1).

Statement 19. A vaccine as stated in any one of the preceding statements wherein the vaccine comprises nanoparticles.

Statement 20. A vaccine as stated in any one of the preceding statements wherein the N:P ratio of amphipathic cell penetrating peptide:mRNA polynucleotide in the vaccine is about 8.5-9.5:1.

Statement 21. A vaccine as stated in any one of the preceding statements further comprising mannose and trehalose.

Statement 22. A vaccine as stated in any one of the preceding statements for use as a therapeutic vaccine.

Statement 23. A vaccine as stated in statements 1-21 for use in the treatment of cancer.

Statement 24. A pharmaceutical composition which comprises a vaccine as stated in statements 1-21 for use in the treatment of sexually transmitted diseases.

Statement 25. A method of treating HPV infection in a warm-blooded animal, such as man, which comprises administering to said animal an effective amount of a vaccine as claimed in in statements 1-21.

What is claimed is:

1. A vaccine, comprising:
i) an mRNA polynucleotide comprising an open reading frame encoding an antigen from an infectious microorganism or an immunogenic fragment thereof; and
ii) an amphipathic cell penetrating peptide comprising or consisting of the amino acid sequence WEARLARALARALARHLARALARALRACEA (SEQ ID_No 1) or a sequence with at least 80% sequence identity or homology.

2. A vaccine as claimed in claim 1 further comprising a second mRNA polynucleotide comprising an open reading frame encoding an antigen from an infectious microorganism or an immunogenic fragment thereof.

3. A vaccine as claimed in claim 2 further comprising a third mRNA polynucleotide comprising an open reading frame encoding an antigen from an infectious microorganism or an immunogenic fragment thereof.

4. A vaccine as claimed in claim 2 or 3 wherein the mRNA polynucleotides are present in about equal % w/w ratio.

5. A vaccine as claimed in any one of the preceding claims wherein the open reading frame encodes an HPV antigen or an immunogenic fragment thereof.

6. A vaccine as claimed in any one of the preceding claims wherein the open reading frame encodes an E1 and/or an E6 and/or an E7 HPV antigen or an immunogenic fragment thereof.

7. A vaccine as claimed in any one of the preceding claims wherein the open reading frame encodes an HPV 6, 8, 11, 16 or 18 antigen or an immunogenic fragment thereof.

8. A vaccine as claimed in any one of the preceding claims wherein the open reading frame encodes an HPV 16 antigen or an immunogenic fragment thereof.

9. A vaccine as claimed in any one of the preceding claims wherein the amphipathic cell penetrating peptide comprises the amino acid sequence WEARLARALARALARHLARALARALRACEA (SEQ ID_No 1).

10. A vaccine as claimed in any one of the preceding claims wherein the amphipathic cell penetrating peptide consists of the amino acid sequence WEARLARALARALARHLARALARALRACEA (SEQ ID_No 1).

11. A vaccine as claimed in any one of claims 1-4 comprising:
i) a first mRNA polynucleotide comprising an open reading frame comprising a sequence selected from SEQ ID_No 2;
ii) a second mRNA polynucleotide comprising an open reading frame comprising a sequence selected from SEQ ID_No 3;
iii) a third mRNA polynucleotide comprising an open reading frame comprising a sequence selected from SEQ ID_No 4;
iv) an amphipathic cell penetrating peptide comprising the amino acid sequence WEARLARALARALARHLARALARALRACEA (SEQ ID_No 1).

12. A vaccine as claimed in any one of claims 1-4 comprising:
i) a first mRNA polynucleotide comprising an open reading frame encoding an antigen selected from SEQ ID_No 5;
ii) a second mRNA polynucleotide comprising an open reading frame encoding an antigen selected from SEQ ID_No 6;
iii) a third mRNA polynucleotide comprising an open reading frame encoding an antigen selected from SEQ ID_No 7;
iv) an amphipathic cell penetrating peptide comprising the amino acid sequence WEARLARALARALARHLARALARALRACEA (SEQ ID_No 1).

13. A vaccine as claimed in any one of the preceding claims wherein the mRNA polynucleotide comprises a five-prime cap (5′ cap).

14. A vaccine as claimed in any one of the preceding claims wherein the mRNA polynucleotide comprises modified uridine nucleotides wherein all uridine nucleotides are N1-methylpseudouridine nucleotides.

15. A vaccine as claimed in any one of the preceding claims wherein the mRNA polynucleotide comprises unmodified cytidine nucleotides.

16. A vaccine as claimed in any one of the preceding claims wherein the vaccine comprises nanoparticles.

17. A vaccine as claimed in any one of the preceding claims wherein the N:P ratio of amphipathic cell penetrating peptide:mRNA polynucleotide in the vaccine is about 8.5-9.5:1.

18. A vaccine as claimed in any one of the preceding claims further comprising trehalose.

19. A vaccine as claimed in any one of the preceding claims comprising
(i) 0.01-1% w/w one or more mRNA polynucleotides comprising open reading frames encoding an HPV antigen or an immunogenic fragment thereof;
(ii) 0.5-2% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 95-99% w/w trehalose.

20. An intravenous formulation comprising a vaccine as claimed in any one of the preceding claims formulated in water.

21. A vaccine as claimed in any one of the preceding claims for use as a therapeutic vaccine.

22. A vaccine as claimed in claims 1-20 for use in the treatment of cancer.

23. A pharmaceutical composition which comprises a vaccine as claimed in claims 1-20 for use in the treatment of sexually transmitted diseases.

24. A method of treating HPV infection in a warm-blooded animal, such as man, which comprises administering to said animal an effective amount of a vaccine as claimed in in claims 1-20.
